(19) United States
(12) Patent Application Publication
(10) Pub. No.: US 2013/0109987 A1
(43) Pub. Date: May 2, 2013

(54) METHOD AND DEVICE FOR TREATMENT OF ARRHYTHMIAS AND OTHER MALADIES

(71) Applicant: MEDICAL DEVICE INNOVATIONS INC., Carlsbad, CA (US)

(72) Inventors: Christopher Gerard Kunis, Escondido, CA (US); Karmi N. Robison, Vista, CA (US); Gerald M. Stobbs, Alpine, CA (US)

(73) Assignee: MEDICAL DEVICE INNOVATIONS INC., Carlsbad, CA (US)

(21) Appl. No.: 13/655,351

(22) Filed: Oct. 18, 2012

Related U.S. Application Data
(63) Continuation-in-part of application No. 13/106,343, filed on May 12, 2011.
Application No. 13/106,343 is a continuation-in-part of application No. 13/324,631, filed on Dec. 13, 2011.
(60) Provisional application No. 61/548,317, filed on Oct. 18, 2011.
(60) Provisional application No. 61/621,666, filed on Apr. 9, 2012.
(60) Provisional application No. 61/648,248, filed on May 17, 2012.
(60) Provisional application No. 61/693,058, filed on Aug. 24, 2012.

(51) Int. Cl. A61B 18/14; A61M 29/00; A61N 1/05; A61B 5/042

(52) U.S. Cl. CPC A61B 18/1492; A61B 5/042; A61M 29/00; A61N 1/059
USPC 600/509; 606/191; 606/41; 607/127

(57) ABSTRACT

Devices and methods are described for treating maladies such as atrial fibrillation. The devices and methods, in some implementations, include one or more rings formed of one or more windings. The rings provide mechanical pressure against an adjacent tissue, e.g., the tissue of a vessel, and the pressure works to inhibit the propagation of electrical signals along the vessel. The windings may be formed of a ribbon. Delivery systems are also provided.

### CROSS-REFERENCE TO RELATED APPLICATIONS

This application claims benefit of priority to the following US Provisional patent applications:
Ser. No. 61/548,317, filed Oct. 18, 2011, entitled “METHOD AND DEVICE FOR TREATMENT OF ARRHYTHMIAS AND OTHER MALADIES”;
Ser. No. 61/621,666, filed Apr. 9, 2012, entitled “METHOD AND DEVICE FOR TREATMENT OF ARRHYTHMIAS AND OTHER MALADIES”;
Ser. No. 61/648,248, filed May 17, 2012, entitled “METHOD AND DEVICE FOR TREATMENT OF ARRHYTHMIAS AND OTHER MALADIES”;
Ser. No. 61/693,058, filed Aug. 24, 2012, entitled “METHOD AND DEVICE FOR TREATMENT OF ARRHYTHMIAS AND OTHER MALADIES”;
   and is a continuation-in-part of co-pending US patent applications:
Ser. No. 13/106,343, filed May 12, 2011, entitled “METHOD AND DEVICE FOR TREATMENT OF ARRHYTHMIAS AND OTHER MALADIES”, and
Ser. No. 13/324,631, filed Dec. 13, 2011, entitled “METHOD AND DEVICE FOR TREATMENT OF ARRHYTHMIAS AND OTHER MALADIES”,
   both of which claim priority to the following US Provisional patent applications:
U.S. Provisional Patent Application Ser. No. 61/334,079, filed May 12, 2010, entitled “Method and Device for Treatment of Arrhythmias and Other Maladies”;
U.S. Provisional Patent Application Ser. No. 61/366,855, filed Jul. 22, 2010, entitled “Method and Device for Treatment of Arrhythmias and Other Maladies”;
U.S. Provisional Patent Application Ser. No. 61/390,102, filed Oct. 5, 2010, entitled “Method and Device for Treatment of Arrhythmias and Other Maladies”;
U.S. Provisional Patent Application Ser. No. 61/443,807, filed Feb. 17, 2011, entitled “Method and Device for Treatment of Arrhythmias and Other Maladies”.

All of the above are owned by the assignee of the present application and are incorporated by reference herein in their entirety.

### BACKGROUND

Atrial fibrillation is a common and dangerous disease. It is the most common arrhythmia, and accounts for approximately ⅓ of all hospitalizations due to heart rhythm disorders. In addition, atrial fibrillation patients have a greatly increased risk of stroke mortality.

The heart's normal sinus rhythm typically begins in the right atrium and proceeds in a single, orderly wavefront at rates of 60 to 100 beats per minute. Atrial fibrillation disrupts normal rhythm. During atrial fibrillation multiple wavefronts circulate rapidly and chaotically through the atria, causing them to contract in an uncoordinated and ineffective manner at rates from 300 to 600 beats per minute. Symptoms arise from the rapid, irregular pulse as well as the loss of cardiac pump function related to uncoordinated atrial contractions. These uncoordinated contractions also allow blood to pool in the atria and may ultimately lead to thromboembolism and stroke.

Initial therapy of atrial fibrillation is usually directed toward reversion to and maintenance of sinus rhythm. Current first-line therapies for atrial fibrillation include the use of anti-arrhythmic drugs and anti-coagulation agents. Anti-coagulation agents can reduce the risk of stroke, but often increase the risk of bleeding. Drugs are useful at reducing symptoms, but often include undesirable side effects. These may include pro-arrhythmia, long-term ineffectiveness, and even an increase in mortality, especially of those with impaired particular function. Drug therapy to slow the ventricular response rate, catheter ablation of the atrioventricular node with pacemaker implantation, or modification of the node without pacemaker implantation can be useful to facilitate ventricular rate control, but thromboembolic risk is unchanged, and therefore the patient must remain on anticoagulants with the problems noted above.

Emerging second-line therapies include surgical and catheter ablation. However, the same are associated with high complication rates, long procedure times, and limited clinical evidence. In addition, their administration typically requires extensive training in the use and installation of complex technology.

Pulmonary vein (PV) isolation is the cornerstone of ablation strategies. The same is currently achieved by causing destructive lesions near the PV. Devices for doing so include point-by-point tip catheters as well as cryoablation devices and radiofrequency ablation devices. However, even the most effective ablation strategies are associated with significant retreatment rates, as well as other difficulties.

The limitations of current medical therapy, repeated cardioversion, either externally or via an implanted device, and atrioventricular node ablation, have caused investigators to search for curative therapy for atrial fibrillation.

### SUMMARY

The present methods and devices are related to implanted devices that have improved safety profiles and which minimize collateral damage over current therapies. Systems and methods are configured to create block in the right or left atria to prevent paroxysmal and/or persistent atrial fibrillation, as well as in the SVC. The implant provides a block for errant electrical conduction to stop physiological drivers in the pulmonary veins from reaching the atria. In some implementations, therapy is delivered within the vessel having a focal tissue effect (as pulmonary vein electrical conductivity occurs endocardially) sufficient to create electrically inert tissue at the point of contact affecting only the implant deployment location, e.g., where ectopic beats occur within the sleeve of the pulmonary vein. No external energy source or capital investment is required for use with this device. Furthermore, there is no need for 3-D mapping for placement, although mapping may be employed and the same may be provided, e.g., by a delivery device itself. The system and method may be especially suited for treating paroxysmal patients and/or patients who have failed a previous RF ablation where micro-reentrant signals have propagated.

Unlike some prior devices, the device and method need not directly integrate into the wall surface of the PVs to obtain isolation, nor is it necessary to cause injury to the tissue via any means of cutting or scoring of atrial or PV cardiac tissue. Rather, in an acute treatment, the device is designed to apply and maintain radial or substantially radial force along a circumference or perimeter or along a helical section of the PVs at the ostium, as well as distal to the ostium, while employing a helical pattern of extension arms, connecting one, two, or more ring-like coils, to disrupt the electrical substrate. In this specification, the device is termed a pulmonary vein isolation device, or “PVID”.

Implementations of the device and method are configured to treat atrial fibrillation without requiring the delivery of energy, without employing needles or other penetrating elements, and without employing elements for scarring. Rather, the device provides mechanical energy against cardiac tissue, e.g., against the intimal lining of the PV, eliminating the electrical refractory process of the myocytes on a cellular level and inhibiting the chemical reaction at the focal site of the implant, thus rendering the tissue electrically inert at the contact point of the implant and creating focal necrosis in a line of block.

The technology may apply mechanical pressure causing a two-step biological response. First, an acute response is caused by pressure-induced apoptosis inhibiting chemical exchange of sodium/calcium and disrupting focal electrical wave propagation. Second, a biological response for chronic or long-term isolation/denervation is provided by causing focal endothelial cell proliferation at the implant site. Of course, other processes may also take place, but the above are believed to be important (though these explanations should not be thought of as limiting in any way the scope of the invention).

A Delivery System Catheter (DSC) is intended to map/pace and isolate the drivers associated with atrial fibrillation that emanate from within the pulmonary veins. The system allows an electrophysiologist (or appropriately trained interventional cardiologists) to identify rapid and complex fractional atrial electrograms (CFAEs) in patients with AF as well as provide an implantable pulmonary vein isolation therapy to achieve normal sinus rhythm. Once a device is implanted, normal sinus rhythm may be confirmed by a mapping capability on the DSC. Such confirmation may occur prior to the time the PVID is released from the delivery device.

The DSC catheters may be sterile single use devices that have a polymeric catheter torque shaft, integral handle which holds and allows implantation of the flexible, metallic implantable device at the distal tip. The catheters are designed to be used with commercially available transseptal sheaths and guidewires. Once the catheters are located within the atrium, the distal segment can be located on the heart wall to perform mapping and pulmonary vein isolation procedures.

Certain attributes of implementations of the DSC & PVID technology may include:

(1) ability to collect intracardiac electrograms for mapping procedures;

(2) ability to deliver pacing stimuli for ECG interrogation and pacing maneuvers;

(3) ability to produce precise block in the pulmonary vein/atria junction to create block that serve as barriers to the conduction of AF; and

(4) compatibility with commercially available transseptal sheaths and or guidewires.

The DSC may have a deflectable distal segment that can be directed to locations in close proximity to the pulmonary veins. In general, the system enables mapping of cardiac tissue along the atria and within the pulmonary veins. Additionally, the DSC also enables the delivery of the PVID to create block at the atrial/PV junction. The block at the pulmonary veins may specifically help to eliminate or reduce the incidence of paroxysmal and other types of atrial fibrillation. The DSC supports delivery of the PVID to all pulmonary veins as well as superior/inferior vena cava, coronary sinus (CS) and other vessels, e.g., for treatment of abdominal aortic aneurysms. The PVID may be designed to prevent arrhythmias from originating in the pulmonary veins. The DSC may include an ECG Interface Cable which provides a means for interrogation of patient intracardiac electrograms prior to and following treatment.

The DSC catheter shaft may include integral wire braiding to enhance torque transfer to the distal tip. Once the physician has located the catheter over the target site, electrode contact of the DSC can be enhanced by advancing the distal deflectable portion of the catheter and pushing into the heart wall. Bi-directional steering of the DSC is controlled by the user via a steering lever on the handle which includes a tension control knob mechanism to hold the deflection angle of the catheter. Each DSC may include multiple electrodes located along the distal loop segment of the catheter. For example, each electrode is (1 mm) long and spacing between electrodes is (5 mm). The electrodes may be arranged in a circular pattern to provide circumferential EGM recordings at and within the PVs. In some implementations, no electrodes or mapping need be included on the distal loop segment. An integral handle is included at the proximal end of the catheter and includes a strain relief/capture device, pull wire or steering wire activation lever and electrical connector for intra cardiac electrogram interrogation.

The DSC distal shape is determined from anatomical literature, physician experience and is designed to conform to the heart wall. The PVID coil wire size may be selected to provide a balance between adequate compliance against the heart wall while providing enough radial force to provide stability to prevent migration and enhance tissue contact when positioned to create a permanent barrier or line of block at the PV/atrial junction.

The DSC is designed to map a large circumferential area within the atrium/PV area such that the physician can deliver the PVID(s) to the appropriate location within the vessel. Paroxysmal atrial fibrillation is believed to often originate in the pulmonary veins, and therefore the PVID may be a valuable tool to create lesions/block in the pulmonary veins to prevent triggers in the pulmonary veins from reaching the left atria.

The catheter attaches to an ECG recorder via connecting cables. A catheter interface cable may be designed to be used in the same manner as other commercially available electrophysiology mapping catheters. The set provides sterile isolation between the catheter and connection(s).

The PVID may employ a novel Nitinol geometry to provide multiple circumferential rings of conduction block at both the ostium and the distal end of the myocardial sleeve located within the vein. The implant's mechanism of action is believed to be bi-modal. In the acute phase, mechanical energy stored in the device applies mechanical pressure to the vein wall, thereby disrupting cell-to-cell ion exchange necessary to support cellular electrical conduction. Over time, the biological response to the implant will produce a long-term electrical blockade as endothelial cells (a principal element of vascular repair) will proliferate which are poor electrical conductors relative to myocardial cells.

The PVID may be constrained in the DSC and delivered to the atrium using standard commercially available transseptal sheaths. Once deployed into the atrium/pulmonary vein, the PVID may take the optimal shape to provide sufficient contact to achieve block of electrical ectopic signals within the PV from entering into the left atrium. These ectopic beats are known to trigger atrial fibrillation.

The PVID is designed to create block at least equal to that of products currently on the market without the use of cryoablation techniques, radiofrequency application, or any other energy source(s). In addition, the physician (end-user) has the advantage of control of the implant for repositioning and ideal implant placement. This allows for the electrophysiologist or interventional cardiologist to tailor the treatment to the needs of each individual patient's anatomy. The physician has full control of both the navigation of the DSC by steering lever and independent control of the implant via the delivery mechanism. This enables the physician to recapture the implant at anytime to reposition the same until such time as deployment and release into the vein is desired. Control and placement of the implant at the ideal location may be done under fluoroscopy, enabling simple and precise deployment of the implant minimizing complications over currently used energy based therapies.

The DSC may be packaged one per carton and may be sterilized by use of Ethylene Oxide (EtO). One or more PVIDs may accompany the DSC in a kit.

The DSC is designed to access the left atrium by means of a percutaneous procedure using a transseptal sheath, and the implant devices are delivered through and using the DSC. A central core wire is used to control delivery of the implant through a lumen of the DSC. Once in the atrium, the catheter may be positioned such that the electrodes on the DSC are in full contact with the atrial/PV wall. The catheter is designed to conform to the cardiac tissue while covering a large area within the atrium/PV. Once in full contact, the system may be used for mapping electrocardiograms to locate any rapid and/or complex fractioned electrograms that may be associated with the occurrence of atrial fibrillation. Several locations may be mapped with the device during the procedure. The DSC will then be used to deploy the implant device within the PV creating a line of block at the Atrial/PV junction. Multiple attempts may be required to accomplish this.

As a result the system is designed to convert the patient's rhythm from atrial fibrillation to normal sinus rhythm. This conversion may be curative in a large percentage of the patients. It is anticipated that many patients will have substantial improvements in reducing the frequency, duration and/or severity of atrial-fibrillation related symptoms.

In one aspect, the invention is directed towards an implantable device for permanently treating atrial fibrillation, including: a device structured and configured for implantation into a mammalian pulmonary vein, the device configured to exert a pressure against a region including the ostium, such that the implantation of the device provides that the pressure against the region including the ostium is substantially consistently greater than zero.

Implementations of the device may include one or more the following. The device may be configured such that the pressure exerted by the device is substantially constant, either over time or over the length of the device, or both. The device may be configured such that the pressure exerted by the device increases as an occurrence of atrial fibrillation decreases and renders the pulmonary vein in which the device is implanted healthier. The pressure exerted may increase by 10-15% over a time period of over three months. In an undeployed configuration, an average diameter of the device may be between about 4 to 60 mm, e.g., 15-45 mm, and every value, to the nearest millimeter, in between. The size of the device may be chosen such that the device is at least 10% oversized, e.g., 20%-40% oversized, compared to a vessel in which it is placed. The device may be configured to deliver a force against adjacent tissue when deployed of between about 0.5 g/mm2 and 340 g/mm2, e.g., of between about 20 g/mm2 and 200 g/mm2. Moreover, the device may be configured to deliver a force against adjacent tissue when deployed of between about 0.04 and 0.2 N/mm2. The proximal ring may be disposed at or adjacent the os and configured to deliver a lesser force when deployed against adjacent tissue than the distal ring. The device may be configured to deliver a force against adjacent tissue when deployed sufficient to cause necrosis or apoptosis in the adjacent tissue, the necrosis or apoptosis sufficient to block or delay electrical conduction traveling along the axis of the vessel. The device may be configured to deliver a force against adjacent tissue when deployed sufficient to compress a K, Ca, or Na channel in the adjacent tissue sufficiently to block or to delay electrical signals traveling along the axis of the vessel. The device may include a microcircuit formed on the device, forming a “smart implant”, which is, e.g., configured to measure or monitor a value of electrical conduction propagating along the axis of the vessel. The microcircuit may be further configured to measure an indication of the patient's heart rhythm. The microcircuit may be further configured to wirelessly transmit the indication of the electrical conduction or patient's heart rhythm. The microcircuit may be further configured to receive an electromagnetic signal and to inductively heat in response to the signal. The microcircuit may be arranged in a circumferential pattern for mapping. Where the implant device is employed to maintain patency of a vessel, microcircuits may be employed to measure flow pressure changes from one end of the implant to the other, providing wireless feedback to a physician about the effect of the implant on patency of the vessel.

It may be preferred to place such a microcircuit on the proximal end of the device, as in some cases distal portions may be too deep in the vein to detect potentials. The proximal portion may in some cases be close to the left atrial tissue, and may pick up signals due to that substrate as well. For these reasons, it may be desired to perform measurement of the signals before implantation, to use as a baseline or index for signals received after implantation. In any case, the circuit may employ electrodes on the tissue contact side of the implant to communicate wirelessly any PV activity that might occur and possibly provide evidence of block being maintained.

A transmitter may be employed to communicate received signals to a receiver such as a smart phone, or in combination with an application running thereon. Such an interface may communicate with the implanted devices that allow simultaneous mapping of each vein to verify block is being maintained and if not, where the conduction is occurring. The vein or veins that are active can then be treated using ablation or another ring, e.g., a single ring system.

The transmitted signal may be indicative of sinus rhythm or a lack thereof, or may indicate other cardiac characteristics. An internal battery may be employed that is rechargeable by the motion of the heart, the motion of the patient, or via an external source. In yet another implementation, the electrical potential of cells may be employed to power or at least recharge the battery. The frequency employed for the communication signals should be chosen properly for medical use. Such circuits may be arranged in a circumferential pattern for mapping, and may further be employed as ICDs. Such circuits may enable controlled resistive heating.

In another aspect, the invention is directed towards a device for determination of post-implantation electrical conduction parameters, including: at least one helical wire or ribbon, the at least one helical wire or ribbon including a flexible circuit including a receiver for reception of signals corresponding to electrical conduction in a pulmonary vein; and a transmitter, the transmitter for transmitting a wireless signal indicative of the received signals.

Implementations of the device may include one or more of the following. The receiver may be the at least one helical wire or ribbon. The transmitter may be configured to transmit two types of signals, a first type of signal corresponding to sinus rhythm, and a second type of signal corresponding to non-sinus rhythm, e.g., atrial fibrillation.

In another aspect, the invention is directed towards an implant device for treating a malady, including: a proximal ring; a distal ring; and an extension arm connecting the proximal ring to the distal ring.

Implementations of the invention may include one or more of the following. The extension arm may include at least one helical winding. The proximal ring and the distal ring may include coils of a ribbon. The radius of the proximal ring may be greater than the radius of the distal ring, or the radii may be equal. Each coil may include at least one winding of the ribbon, e.g., at least 1.5 windings of the ribbon. Each coil may include a pressure feature such as a ridge. In an undeployed configuration, the radius of the proximal ring may be between about 4 to 60 mm and the radius of the distal ring may be between about 6 to 60 mm. In a deployed configuration, the radius of the proximal ring may be between about 2 to 40 mm and the radius of the distal ring may be between about 3 to 40 mm. The rings may be configured to deliver a force against adjacent tissue when deployed of between about 5 g/mm2 and 340 g/mm2, e.g., between about 20 g/mm2 and 200 g/mm2, e.g., between about 0.02 N/mm2 and 0.4 N/mm2. The proximal ring may be configured to deliver a lesser force when deployed against adjacent tissue than the distal ring. The width of the ribbon may be between about 0.25 and 2.5 mm, e.g., 1 and 2 mm. An extremity of the ring may be shaped to increase frictional or mechanical resistance against movement, e.g., may be shaped to include scallops, ribs, or a club shaped end. One or both extremities of the ribbon may be fashioned with a ball shaped end to promote non-perforation. The implant device may be coated with a material composition, surface treatment, coating, or biological agent and/or drug.

In another aspect, the invention is directed towards a method of providing a therapy for atrial fibrillation over time, including: implanting a device into a pulmonary vein, the implanted device oversized and thus configured to exert a pressure against the region including the ostium and a portion of the pulmonary vein; and such that the implantation provides that the pressure against the region including the ostium and a portion of the pulmonary vein is substantially consistently greater than zero.

In another aspect, the invention is directed towards a method for intraoperative treatment of atrial fibrillation, including: during an open-heart surgery, implanting a device into a pulmonary vein, the implanted device oversized and thus configured to exert a pressure against the region including the ostium and a portion of the pulmonary vein; and such that the implantation provides that the pressure against the region including the ostium and a portion of the pulmonary vein is substantially consistently greater than zero, e.g., sufficient to allow the device to maintain its position within the vein.

In another aspect, the invention is directed towards a method for determining propriety of implant installation configuration prior to release from a delivery device, the implant for treatment of atrial fibrillation, including: detecting a first level of conduction along a pulmonary vein; implanting a device at least partially into the pulmonary vein through a delivery device, the implanted device oversized and thus configured to exert a pressure against the region including a portion of the pulmonary vein, the device to be implanted coupled to a central core or pusher wire, the pusher wire configured to hold the device against relative movement of the delivery device at a location at least partially in a pulmonary vein; detecting a second level of conduction along a pulmonary vein; and if the second level is sufficiently below the first level, causing the device to separate from the pusher wire; and if the second level is not sufficiently below the first level, using the pusher wire to change the position of the device at least partially within the pulmonary vein.

It another aspect, the invention is directed towards a method for determining propriety of implant installation configuration prior to release from a delivery device, the implant for treatment of atrial fibrillation, including: implanting a device at least partially into the pulmonary vein through a delivery device, the implanted device oversized and thus configured to exert a pressure against the region including the ostium and a portion of the pulmonary vein, the device to be implanted coupled to a central core or pusher wire, the pusher wire configured to hold the device against relative movement of the delivery device at a location at least partially in a pulmonary vein; detecting an orientation of the implanted device relative to the pulmonary vein; and if the orientation of the implanted device is appropriate relative to the pulmonary vein, e.g., if the plane of the ring is substantially perpendicular to the axis of the vessel, e.g., to within 30°, causing the device to separate from the pusher wire; and if the orientation of the implanted device is not appropriate relative to the pulmonary vein, using the pusher wire to change the position of the device at least partially within the pulmonary vein.

Implementations of the method may include one or more of the following. The device may include a single ring having one or more windings or a dual ring system. If a dual ring system, the device includes a proximal ring, a distal ring, and an extension arm between the proximal and distal ring, and where the orientation is determined to be appropriate if the rings are perpendicular to the axis of the pulmonary vein or within 30° of being perpendicular to the axis of the pulmonary vein. The method may further include using fluoroscopy to determine the orientation of the implanted device. Each ring may include one or more windings or coils of the ribbon.

In another aspect, the invention is directed towards a method for determination of post-implantation electrical conduction parameters, including: implanting at least one helical wire or ribbon in a pulmonary vein, the at least one helical wire or ribbon including a flexible circuit including a receiver for reception of signals corresponding to electrical conduction in a pulmonary vein, the flexible circuit further including a transmitter for transmitting a wireless signal indicative of the received signals; receiving a signal transmitted wirelessly from the transmitter, and rendering a result corresponding to the received signal on a display. In one optional implementation, the result may indicate sinus rhythm or non-sinus rhythm.

In another aspect, the invention is directed towards a method for treating a malady, including: inserting an implant device into a vessel of the patient, the vessel substantially defining a longitudinal axis, the implant device including a proximal ring substantial defining a proximal plane, a distal ring substantially defining a distal plane, and an extension arm connecting the proximal ring to the distal ring; such that the inserting includes inserting the implant device such that a proximal angle between the proximal plane and the longitudinal axis is 90 degrees plus or minus 30 degrees, and such that a distal angle between the distal plane and the longitudinal axis is 90 degrees plus or minus 30 degrees.

Implementations of the method may include one or more of the following. The method may further include measuring the angle of the rings using fluoroscopy. The malady may be atrial fibrillation and the vessel may be a pulmonary vein, and the method may further include measuring a first value of the electrical conduction along the pulmonary vein prior to the inserting, and measuring a second value of the electrical conduction along the pulmonary vein subsequent to the inserting, and if the second value is not sufficiently below the first, then performing one or more of the below steps: installing a touchup ring into the pulmonary vein; re-inserting the implant device into the pulmonary vein; performing a step of ablating the pulmonary vein where the ablating is performed using RF or cryoablation; or inductively heating the implant device to cause necrosis or apoptosis of adjacent tissue.

In another aspect, the invention is directed towards a method for installing an implant, including feeding an implant into a delivery lumen of a delivery device, the implant including at least one helical wire or ribbon, the helical wire or ribbon associated with a twist direction, the delivery device including a proximal end and a distal end; disposing the distal end of the delivery device at a delivery location; pushing the implant through the delivery lumen using a central core or pushing device coupled at a distal end of the pushing device to the implant; pushing the implant such that the implant exits the distal end of the delivery device but is still attached to the pushing device; and twisting the pushing device an angular amount greater than 10°, the twist having a direction opposite that associated with the helical wire or ribbon.

Implementations of the invention may include one or more following. The helical wire or ribbon may be formed of a ribbon having a width of between 0.25 and 2.5 mm. The delivery location may be a mammalian pulmonary vein. The angular amount may be less than 90°, and may further be between about 3-5%. The central core or pushing device may include a universal joint, the universal joint configured to allow two degrees of freedom when the distal end of the pushing device is distal to or adjacent the distal end of the delivery device, the two degrees of freedom not including an azimuthal rotation angle associated with the twist.

In another aspect, the invention is directed towards a method for assisting patency of a vessel, including implanting a device at least partially into a vessel through a delivery device, the device including a proximal ring, a distal ring, and an extension arm between the proximal and distal ring, and where the implanting is such that the rings are perpendicular to the axis of the vessel or within 30° of being perpendicular to the axis of the vessel. A single ring system may also be employed to serve the cause of patency.

In another aspect, the invention is directed towards a method for treating atrial fibrillation, including implanting a device at least partially into a left atrial substrate of a patient through a delivery device, the device including a proximal ring, a distal ring, and an extension arm between the proximal and distal ring.

In another aspect, the invention is directed towards a method for treating a malady, including: choosing a size of an implant device for insertion into a vessel of a patient, the implant device including a proximal ring, a distal ring, and an extension arm connecting the proximal ring to the distal ring; and inserting the implant device into the vessel of the patient, such that the choosing includes selecting a size of the distal ring of the implant device to be about 10-50% oversized compared to the size of the vessel.

Implementations of the method may include one or more of the following The method may further include selecting a size of the distal ring of the implant device to be about 10-50% oversized compared to the size of the vessel, e.g., about 30-40% oversized compared to the size of the vessel.

In another aspect, the invention is directed towards a method for treating a malady, including: choosing a size of an implant device for insertion into a vessel of a patient, the implant device including a proximal ring, a distal ring, and an extension arm connecting the proximal ring to the distal ring; inserting the implant device into the vessel of the patient, such that the choosing includes selecting the size of the implant device such that the implant device compresses a K, Ca, or Na channel in adjacent tissue sufficiently to block or to delay electrical signals traveling along the axis of the vessel.

Implementations of the invention may include one or more of the following. The inserting may include delivering the implant to the vessel through a catheter including a pigtail distal end. The vessel may be a pulmonary vein. The method may further include mapping at least one pulmonary vein and/or ablating at least one pulmonary vein. The ablating may be performed using at least one electrode disposed on a delivery device. The inserting may include delivering the distal ring into the pulmonary vein and delivering the proximal ring into the ostium of the pulmonary vein. The inserting may further include pushing the implant device through the catheter with a pushing mechanism or means, which may be a central core wire. The pushing mechanism means may be coupled to the implant device using a grabbing means. The method may further include administering local anesthesia and not general anesthesia to the patient. The mapping may include determining the sizes of at least two pulmonary veins, and may further include delivering at least one implant device to each pulmonary vein. The method may further include loading implant devices into the delivery device in the order in which they are to be successively implanted in pulmonary veins. The malady may be atrial fibrillation or vessel non-patency. The method may further include inducing a local heating effect to be present on the implant device by induction, RF, or other electromagnetic means. The method may further include recapturing the implant device after the inserting. The compression of the K, Ca, or Na channel in adjacent tissue sufficiently to block electrical signals traveling along the axis of the vessel may include compressing the first one to five cellular layers of the adjacent tissue. The mapping may be performed both before the inserting and after the inserting. The compression may be such that the delay is caused in conduction of at least 50%.

In another aspect, the invention is directed to a method for treating a malady, including: choosing a size of an implant device for insertion into a vessel of a patient, the implant device including a proximal ring, a distal ring, and an extension arm connecting the proximal ring to the distal ring; and inserting the implant device into the vessel of the patient, such that the choosing includes selecting the size of the implant device such that the implant device causes a necrosis in adjacent tissue sufficient to block electrical signals traveling along the axis of the vessel.

In another aspect, the invention is directed to a method for treating a malady, including: choosing a size of an implant device for insertion into a vessel of a patient, the implant device including a proximal ring, a distal ring, and an extension arm connecting the proximal ring to the distal ring; inserting the implant device into the vessel of the patient, such that the choosing includes selecting a diameter of the distal ring of the implant device to be at least 1.1 to 2 times the diameter of the vessel (or other values as have been disclosed herein). The choosing may further include selecting an implant size according to a sizing scheme. It will be understood that the term “inserting” may include pushing the implant in a distal direction out of a delivery device as well as removing a delivery device in a proximal direction, and thereby deploying the implant with no distal force applied from the implant to the tissue. Generally the latter technique will yield superior outcomes.

Implementations of the invention may include one or more of the following. The method may further include selecting a radius of the distal ring of the implant device to be at least five times the radius of the vessel.

In another aspect, the invention is directed to a method for treating a malady, including: inserting a catheter into a vessel of a patient, the catheter having loaded within an anchoring device for partial insertion into a vessel of a patient, the anchoring device including at least a distal ring; partially extending the distal ring from the catheter such that the distal ring is anchored in the vessel; activating at least one electrode on the catheter, the at least one electrode substantially adjacent to tissue when the distal ring is anchored in the vessel, the activating causing ablation and necrosis of the adjacent tissue; retracting the distal ring into the catheter; and withdrawing the catheter.

Implementations of the invention may include one or more of the following. The method may further include activating a plurality of electrodes on the catheter, e.g., a distal end of the DSC, the electrodes distributed along the pigtail distal end. The method may further include rotating the catheter at least partially during the activating, thereby causing ablation and necrosis of tissue and the creation of partial circumferential linear lesions. The method may further include inserting an implant device into the vessel, the implant device including a proximal ring, a distal ring, and an extension arm between the proximal and distal ring.

In another aspect, the invention is directed towards a delivery device for implanting and allowing manipulation of an implant, the implant for treating a malady, the delivery device including: a catheter including a delivery lumen, the delivery lumen extending from a catheter proximal end to a catheter distal end; a central core or pusher configured for insertion into the delivery lumen, the pusher including a distal end, the distal end of the pusher including a device for securing an implant, e.g., a hook, or grabber, or a universal joint, the universal joint allowing no additional degrees of freedom when the universal joint is within and not adjacent to the catheter distal end, the universal joint allowing at least two additional degrees of freedom when the universal joint is outside of or adjacent to the catheter distal end.

Implementations of the invention may include one or more of the following. The device for securing the implant may include a boss that, together with an inner wall of the lumen of the DSC through which the PVID is delivered, holds the PVID securely to the central core wire. When outside the inner wall, the PVID proximal end springs away from the boss and is thus released therefrom. In an alternative implementation, two such central core wires are employed, one with a boss securing a distal end of the PVID and one with a boss securing the proximal end. The central core wire may push the PVID out a side port. The device for securing an implant may include a jawbone structure which is closed when the distal end of the pusher is within the delivery lumen and open when the distal end of the pusher is outside the delivery lumen, and where the implant includes a half-dog bone shape which is inserted within the jawbone structure during the securing. The jawbone may include a boss in a lip of the jawbone, the boss structured and configured that the implant can only be secured to the jawbone in one configuration. The jawbone may include a boss in a lip of the jawbone, the boss structured and configured that the implant can only be secured to the jawbone in two configurations. The pusher or central core may include a wire attachable to the implant, such that electrical energy applied to the wire causes breakage of the wire, thus separating the implant from the pusher. The delivery lumen may be configured to allow placement of at least two pushers and respective implants therein. The delivery lumen may be configured to allow placement of a cartridge therein, the cartridge containing at least two pushers and respective implants. The catheter distal end may further include electrodes for RF ablation or mapping. The catheter may be configured to provide RF ablation or mapping through the implant.

In another aspect, the invention is directed towards a delivery device for implanting and allowing manipulation of an implant, the implant for treating a malady, the delivery device including: a catheter including a delivery lumen, the delivery lumen extending from a catheter proximal end; the catheter further including a straight or pigtail section through which the delivery lumen extends, and if a pigtail section, then the pigtail section may be straight and collinear with the catheter during delivery and configurable into a pigtail during deployment of the implant.

Implementations of the invention may include one or more of the following. The pigtail section may be located at a distal end of the catheter, or located proximal to a distal end of the catheter. A radial size of the pigtail section may be adjustable using a lever or knob on a handle of the catheter, the handle located at a proximal end of the catheter. A maximum radial size of the pigtail section may be configured to be 15 mm to 25 mm. The catheter and pigtail section may be configured such that deployment of the implant in a vessel leads to an axis of the implant being substantially parallel to an axis of the vessel, where substantially parallel is between about 0 and 30°. The pigtail section may further include electrodes for RF ablation or mapping. The catheter itself may also be configured to provide RF ablation or mapping through the implant.

In another aspect, the invention is directed towards a kit for treating a malady by deploying an implant device in a vessel, including: a device structured and configured for implantation into a mammalian pulmonary vein, the device configured to exert a pressure against a region including the ostium, such that the implantation of the device provides that the pressure against the region including the ostium is substantially consistently greater than zero; and a delivery system, such that upon deployment from the delivery system, the implant device is disposed within a target vessel.

Implementations of the kit may include one or more of the following. The delivery system may include a catheter with a straight distal end or a distal end with a pigtail section. The kit may further include a touchup ring. The touchup ring may be a device described in this specification, e.g., a single or double ring device. The touchup ring may be a ribbon in a helical shape having at least one winding.

In another aspect, the invention is directed towards a kit for treating a malady by deploying an implant device in a vessel, including: a device structured and configured for implantation into a mammalian pulmonary vein; and a delivery device for implanting and allowing manipulation of the implanted device, the implanted device for treating a malady, the delivery device including a catheter including a delivery lumen, the delivery lumen extending from a catheter proximal end, the catheter further including a straight or pigtail section through which the delivery lumen extends. If a pigtail section, the pigtail section is collinear with the catheter during delivery and configurable into a pigtail during deployment of the implanted device.

In another aspect, the invention is directed towards a kit for treating a malady by deploying an implant device in a vessel, including the above-noted implant device, and a delivery system, the delivery system including a catheter having a pigtail distal end, such that upon deployment of the implant device from the pigtail distal end, a longitudinal axis of the implant device is substantially collinear with a longitudinal axis of the vessel. Due to a nature of the implant to self-right, straight delivery devices may also be employed. The tendency to self-right is believed to be due to the ring and winding structure.

Advantages of the invention may include one or more of the following. The device can be deployed into the target zone, e.g., into the PV, where cryoablation and radio frequency ablation techniques cannot. Devices may be employed to provide multiple locations of circumferential block as well as lateral disruption along the PV sleeve to dissociate ectopic beats that emulate from within the PVs. The device may be delivered using a procedure under only local anesthesia rather than requiring general anesthesia. The design of implementations of the implant allow for a substantially equal distribution of circumferential force along the device, minimizing variables related to procedural complications such as were encountered in the prior art. Furthermore, as the ends of the implanted device are not confined, the device adjusts itself, radially distributing load dynamically along the length of the device. Such load distribution helps the desirable effect of a lack of migration of the implant. The pressure mediated block creates multiple rings of block including at proximal and distal ends of the PV sleeve.

Other advantages may be apparent from the description that follows, including the claims and figures.

### BRIEF DESCRIPTION OF THE DRAWINGS

FIG. 1 schematically illustrates an implant device according to an exemplary arrangement of the present invention in which two rings, each having a set of windings or coils, are separated by an extension arm.

FIG. 2 illustrates an implant device according to an exemplary arrangement of the present invention having a single ring, the ring having a set of windings or coils.

FIG. 3 schematically illustrates the implant device of FIG. 1 situated at the os of a pulmonary vein.

FIG. 4 schematically illustrates a delivery device situating an implant within the pulmonary vein of a heart according to an exemplary arrangement of the present invention.

FIG. 5 schematically illustrates a delivery device with an implant partially deployed according to an exemplary arrangement of the present invention.

FIG. 6 schematically illustrates an implant providing pressure against the inner wall of the pulmonary vein according to an exemplary arrangement of the present invention.

FIGS. 7 (A) and (B) illustrate different types of implant devices, such implant devices including a two rings, according to exemplary arrangements of the present invention. It will be understood that implant devices including just one ring or more than two rings are also encompassed by the scope of this specification.

FIG. 8 illustrates an implant device with two rings according to an exemplary arrangement of the present invention.

FIG. 9 illustrates an implant sizing guide according to an exemplary arrangement of the present invention.

FIG. 10 illustrates an exemplary device according to an arrangement of the present invention for measuring the size of a vessel.

FIGS. 11(A)-(C) illustrate use of single and dual ring systems within a bifurcated pulmonary vein system according to an exemplary arrangement of the present invention.

FIG. 12 illustrates an exemplary DSC according to an arrangement of the present invention.

FIGS. 13A-13D illustrate steps of deployment of an implant device from a DSC having a pigtail distal tip according to an exemplary arrangement of the present invention.

FIGS. 14A-14C illustrate portions of a DSC which may be employed to hold and deploy an implant according to an exemplary arrangement of the present invention.

FIGS. 15, 16, and 17 illustrate portions of another type of DSC according to an exemplary arrangement of the present invention which may be employed to hold and deploy an implant.

FIG. 18 illustrates an implant with a keyway according to an exemplary arrangement of the present invention.

FIG. 19 illustrates an implant with the keyway being held by a DSC according to an exemplary arrangement of the present invention.

FIG. 20 illustrates another implant with a keyway according to an exemplary arrangement of the present invention.

FIG. 21 illustrates an alternative implant with no keyway according to an exemplary arrangement of the present invention being held by a DSC.

FIG. 22 illustrates a perspective view of a handle according to an exemplary arrangement of the present invention which may be employed to deploy an implant.

FIG. 23 illustrates a cutaway portion of a handle according to an exemplary arrangement of the present invention which may be employed to deploy an implant.

FIG. 24 illustrates a handle according to an exemplary arrangement of the present invention which may be employed to deploying an implant, with an implant almost completely deployed.

FIGS. 25A-25C illustrate alternative distal portions of the DSC, with a side port through which an implant is deployed.

FIG. 25D illustrates an alternative distal portion of the DSC, with a split section through which an implant is deployed, allowing control of both proximal and distal ends of the implant.

FIGS. 26-28 illustrate an alternative implementation of an implant according to an exemplary arrangement of the present invention.

FIGS. 29A and 29B illustrate related alternative implementations of a DSC according to exemplary arrangements of the present invention.

FIG. 30 illustrates an exemplary portion of a DSC according to an exemplary arrangement of the present invention.

FIG. 31 illustrates an exemplary material which may be employed to create an implant according to an exemplary arrangement of the present invention.

FIG. 32 is a flowchart illustrating an exemplary method of using the DSC and implant according to an arrangement of the present invention.

FIG. 33 is another flowchart illustrating an exemplary method of using the DSC and implant according to an arrangement of the present invention.

FIG. 34 schematically illustrates an implant device according to an exemplary arrangement of the present invention within a vessel, e.g., a pulmonary vein.

FIGS. 35 (A)-(C) illustrate various views of the implant device of FIG. 34, with a single helix connecting two coils or rings, according to an exemplary arrangement of the present invention.

FIGS. 36 (A)-(C) illustrate various views of another embodiment of the implant device, illustrating how two helices or a dual helix system may be employed to connect two coils or rings, according to an exemplary arrangement of the present invention.

FIGS. 37 (A)-(B) illustrates features that may be employed in certain implementations of the implant device, according to arrangements of the present invention.

FIG. 38 illustrates a feature that may be employed in certain implementations of the implant device, according to an exemplary arrangement of the present invention.

FIG. 39 illustrates a feature that may be employed in certain implementations of the implant device, according to an exemplary arrangement of the present invention.

FIG. 40 illustrates details of a delivery device that may be employed to deliver the implant device, according to an exemplary arrangement of the present invention.

FIG. 41 illustrates details of the device of FIG. 40.

FIG. 42 illustrates additional details of the device of FIG. 40.

FIG. 43 illustrates a perspective view of the device of FIG. 40.

FIGS. 44 (A)-(C) illustrate proximal, distal end, and distal tip details of the device of FIG. 40.

FIG. 45 (A) illustrates a terminal end of an implant device, showing the end which may be grabbed by a grabber associated with the delivery device, or with a retrieval device, according to an exemplary arrangement of the present invention. FIG. 45 (B) illustrates the grabber associated with the delivery device, or with a retrieval device, according to an exemplary arrangement of the present invention.

FIG. 46 schematically illustrates an implant device as well as a delivery device that may be used for implantation, according to an exemplary arrangement of the present invention.

FIGS. 47(A) and (B) illustrate a grabber device, in both a closed and opened configuration, respectively, according to an exemplary arrangement of the present invention.

FIG. 48 illustrates a system having a similar configuration as the implant device but which may be employed to ablate tissue using radio frequencies, according to an exemplary arrangement of the present invention.

FIGS. 49 (A) and (B) illustrate views of another embodiment of the system of FIG. 48. FIG. 49 (A) illustrates the device in a vein and FIG. 49 (B) illustrates necrosed tissue patterns that may be created.

FIG. 50A illustrates removal of the implant device from a delivery device using a pusher and ratchet sleeve, according to an exemplary arrangement of the present invention.

FIG. 50B illustrates a ratchet sleeve that may be employed to remove the implant device from a delivery device, according to an exemplary arrangement of the present invention.

FIGS. 51 (A)-(D) illustrate steps in removing the implant device from one embodiment of a delivery device, where the implant device expands off a mandrel, according to an exemplary arrangement of the present invention.

FIGS. 52 (A)-(D) illustrate steps in removing the implant device from another embodiment of a delivery device, where the implant device is deployed from a tube, according to an exemplary arrangement of the present invention.

FIGS. 53-54 illustrate how implant devices may be used to secure a sleeve for treatment of abdominal aortic aneurysms.

FIGS. 55(A)-(B) illustrate details of an alternative delivery system.

FIG. 56 illustrates a perspective view of the delivery system of FIG. 55.

FIG. 57 illustrates a handle feature in the delivery system of FIG. 55.

FIG. 58 illustrates an exemplary handle assembly in the delivery system of FIG. 55.

FIG. 59 illustrates the system of FIG. 55 with an implant partially deployed.

FIGS. 60-62 illustrate exemplary views of an alternative implant end fixation device for use in the delivery system of FIG. 55.

FIG. 63 illustrates an exemplary curved ribbon which may be employed in a PVID implant.

FIGS. 64-66 illustrate an alternative implementation of a delivery system catheter.

FIGS. 67-69 illustrate an alternative implementation of a delivery system catheter.

FIGS. 70-72 illustrate an alternative implementation of a delivery system catheter.

Like reference numerals refer to like elements throughout.

### DETAILED DESCRIPTION

### Implant Device

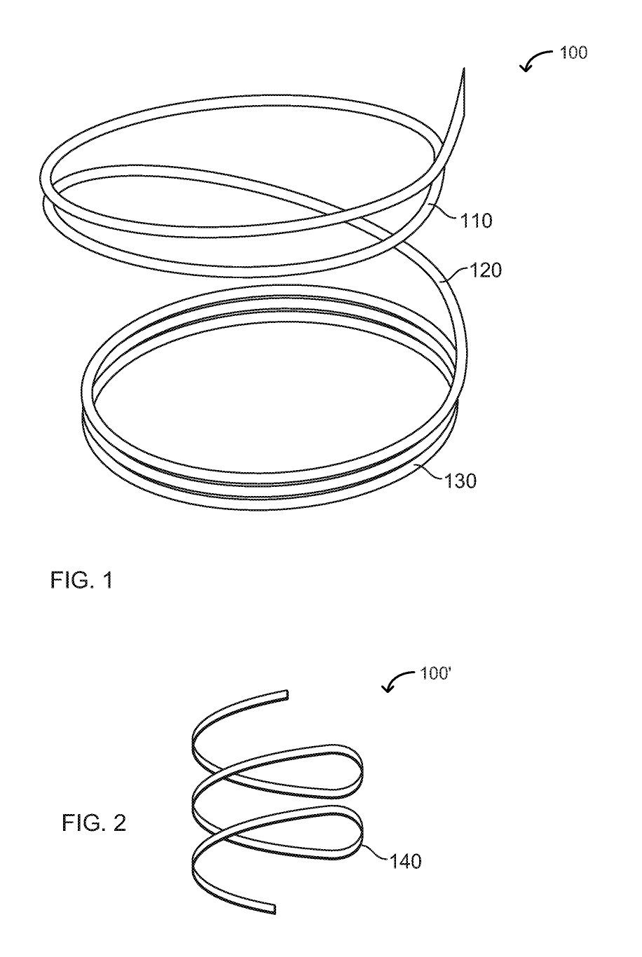

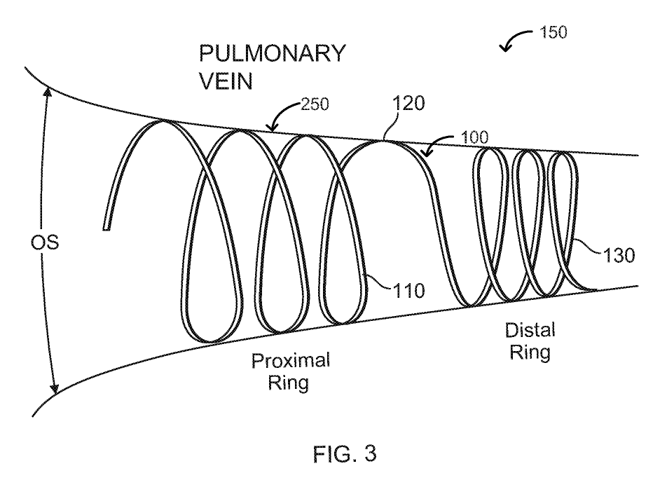

Referring to FIG. 1, an implant or implant device 100 or PVID (the terms are used interchangeably) may include a first ring 110, a second ring 130, and an extension arm 120 connecting the first ring to the second ring. The extension arm 120 is generally helical, although other shapes including straight connectors may also be employed. FIG. 2 illustrates an alternative PVID 100′ in which a single ring system is employed.

In one implementation, the PVID 100 may include two separated rings, each composed of a portion of a coil or winding, a single coil or winding, or multiple coils or windings, that are connected by a single helical wire, a double helical wire, or a set of multiple helical wires. Generally a single helical wire has been found particularly useful. Such an implant, in place within a vessel such as the PV, is illustrated schematically in FIG. 1 as well as in FIGS. 35-37. In this disclosure, the term “ring” or “ring system” is used. However, it will be understood that the same does not necessarily refer to a closed ring, and that the terms are in most cases used to refer to a wire or ribbon wound in a helical configuration, where each “ring” is one to several turns of the wire, closely spaced. The extension arm, or that which connects the two rings, may be also helical, but is generally less closely spaced. In other words, the pitch of the windings or coils within a ring, e.g., 1-10 mm pitch per turn, is less generally than the pitch of the extension arm, e.g., 10-50 mm pitch per turn.

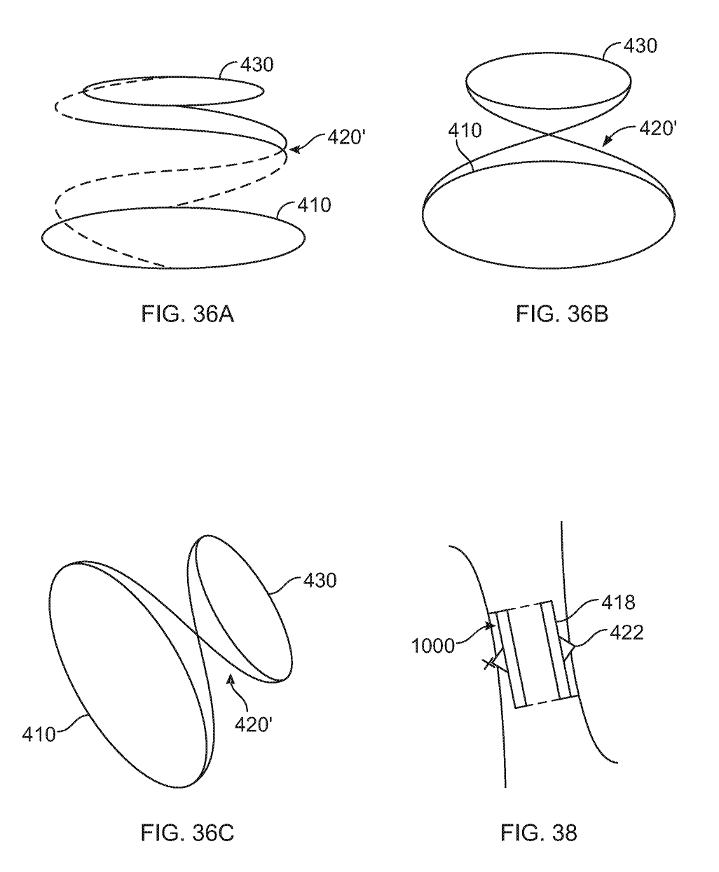

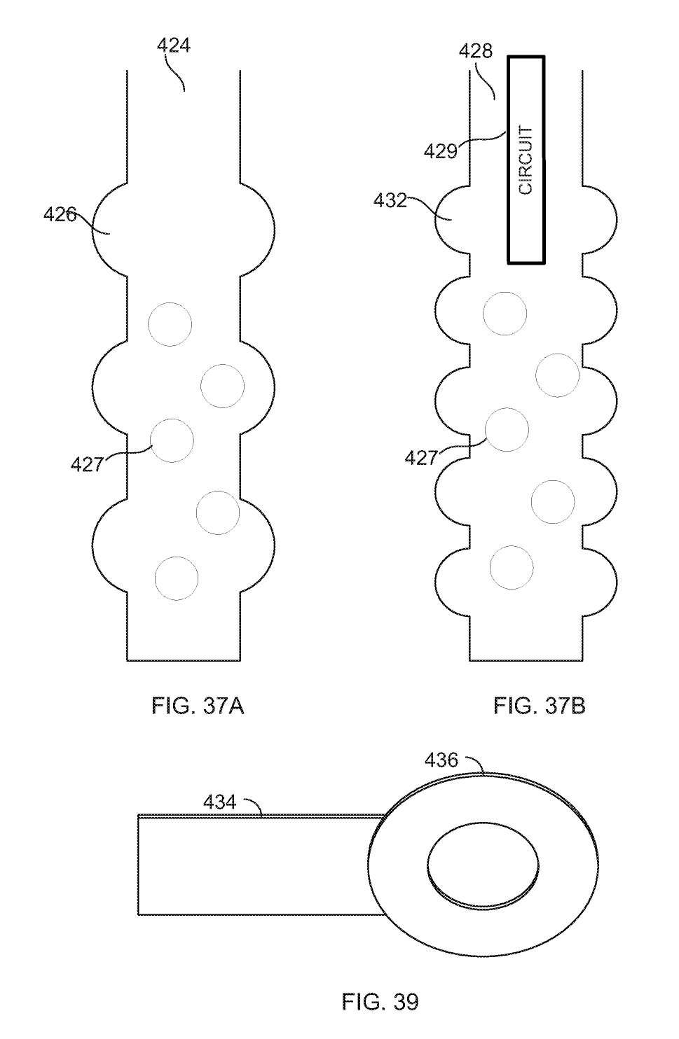

Referring to FIG. 3, a system 150 is shown in which an implant device 100 is illustrated schematically within a pulmonary vein 250. The implant device 100 includes a proximal ring 110 placed at the os or adjacent the os in the pulmonary vein (PV), a distal ring 130 placed deeper in the pulmonary vein, and the two are separated by a helix or helical wind 120. FIGS. 35-36 illustrate various views of the implant device of FIG. 1, where a single helical wind 420 is employed between the rings 410 and 430. FIG. 37 illustrates the situation where a double helical wind 420′ is employed between rings 410 and 430. It is noted that in the system of FIG. 37, the implant may be placed in a straight and undeployed configuration by simply pulling the ring 410 away from the ring 430.

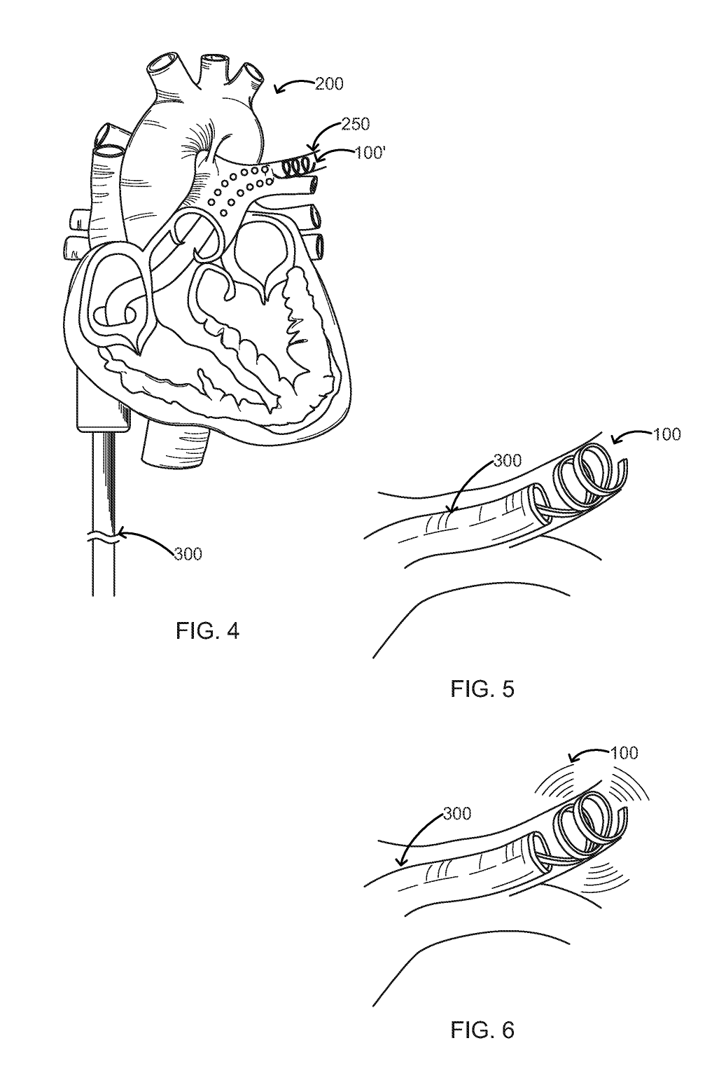

Referring to FIG. 4, a DSC 300 transports the implant or PVID 100 or 100′ to a location in the left atrium of the heart 200, and in particular into a pulmonary vein 250. Referring to FIG. 5, the implant 100 is then deployed from the distal tip of the DSC 300, and as shown in FIG. 6, exerts pressure against an inner wall of the pulmonary vein 250. As has been described, such pressure, properly modulated, creates a conductive block and isolates the PV from the atrium. By placing an implant in each of the pulmonary veins, aberrant electrical signals emanating from the pulmonary veins may be effectively blocked from reaching the heart. Such pulmonary vein isolation is believed to be highly accurate and therapeutic in treating atrial fibrillation.

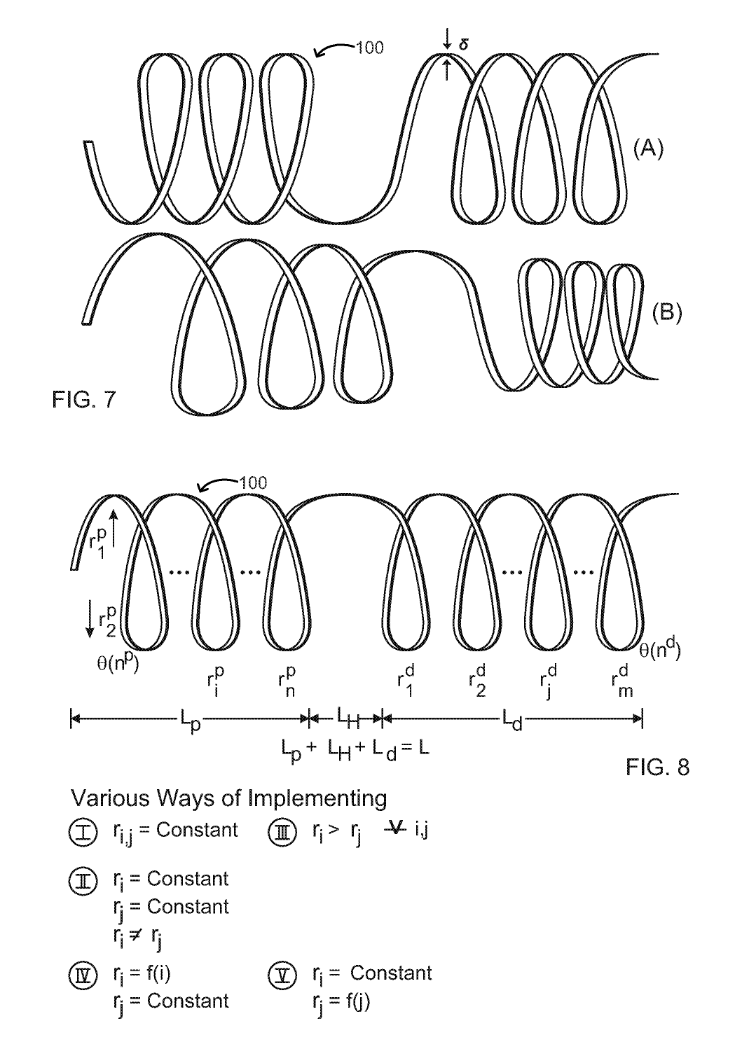

FIG. 7 illustrates an exemplary implant 100. In FIG. 7(A), the implant 100 is a dual ring system comprising a ribbon that forms two rings, each with its own set of coils or windings. The thickness of the ribbon is illustrated as δ. FIG. 7(A) illustrates a symmetric system, where each ring has the same diameter. FIG. 7(B) illustrates an asymmetric system, in which the ring diameters differ.

FIG. 8 illustrates the general situation, for the device 100. While the device 100, a dual ring system, is illustrated, it will be understood that the same general considerations also applied to a single ring system 100′, as well as ring systems with more than two rings.

In FIG. 8, the first ring is illustrated as r′, arbitrarily assigned as the proximal ring, and the second as r^{d}, arbitrarily assigned as the distal ring. Each coil or winding within each ring is enumerated by a number. So the first coil within the proximal ring has a radius r^{p}₁, the second r^{p}₂, etc. Similar enumerations are indicated for the distal ring. Each ring may have less than 1 coil, 1 coil, 1.5 coils, 2 coils, 2.5 coils, for coils, or more.

The proximal ring has a length Lₚ, and the distal ring has a length L_{d}. The length of the extension arm is indicated as L_{H}. As may be seen, a total length L=Lₚ+L_{d}+L_{H}.

The pitch of each ring may be defined as the number of turns n/Lₚ (proximal) and m/L_{d} (distal). A pitch of the extension arm may also be defined, as the number of turns in the extension arm divided by L_{H}. The lengths of the sections can vary according to the flexibility in pitch allowed by the material, and how the physician installs the device. For example, the physician may install the device in a highly compressed state, a highly extended state, or a state in-between.

The ribbon forming the device 100 may also in general be angled as illustrated. While the angles θ(n^{p}) and θ(n^{d}) may imply a constant angle, at least for each ring, each coil may also be designed to have its own appropriate angle. Such angling may be employed to create a better attachment to the lining of the vessel in which the device is situated. In general, it is been found satisfactory results may be obtained for θ such that the ribbon is parallel to the wall. However, such angling may become especially important for the purchase of the proximal ring in the os, as the radius of the os generally changes quickly with respect to position along the axis of the pulmonary vein. Such angling may also be particularly important in the case of single ring systems, where less anchoring may be present. Nevertheless, useful single ring systems may include those with 2-3 coils constituting the ring. In many cases, the coils may increase in diameter to form a “tornado” shape, in which case the distal ring is typically the smallest. In even more advanced designs, the pitch of the coils may vary, larger pitches generally being associated with increased stability. Like the dual ring systems, various sizes may be provided to accommodate varying vasculature, e.g., 10-12 sizes may be provided, varying from 10-45 mm in diameter.

Various ways of arranging the above-noted variables are also illustrated in FIG. 8. For example, in arrangement I, all of the radii are constant. In arrangement II, all of the radii within a ring are constant, but the proximal ring radius differs from that of the distal ring. In arrangement III, all of the radii of the proximal ring are greater than all of the radii of the distal ring, and there is a decrease of radius in the direction proximal to distal within each ring. In arrangement IV, the radii of the proximal ring vary, but those of the distal ring are constant, e.g., thereby providing primarily an anchoring arrangement. In arrangement V, the roles are switched from that of arrangement IV. It will be understood that the above are merely exemplary, and that other variations may also be appropriate to tailor sizing to a particular patient's anatomy. For example, r^{p}ᵢ>r^{p}ⱼ for all i<j, and the same may also be true for the distal radii. As another different example, r^{p,d}ᵢ>r^{p,d}ᵢ₊₁. In another example, r^{p}ᵢ>r^{p}ᵢ₊₁ but r^{d}ᵢ<r^{d}ᵢ₊₁. In addition, combinations of the above arrangements may in some cases be employed.

The diameter of the undeployed coils may be about 4 mm to 60 mm for the proximal coil, and about 6 mm to 60 mm for the distal coil, e.g., about 15-50 mm diameter, and in all cases may take on every value in between the ranges, e.g., per every 1 mm. The diameter of the deployed coils may be about 2 mm to 40 mm for the proximal coil, and about 3 mm to 40 mm for the distal coil. Sizes found particularly useful include those disclosed in the table below:

| | Proximal | Distal | | | |
|---|---|---|---|---|---|
| | Ring | Ring | | Ribbon | Ribbon |
| Implant | Diameter | Diameter | Length | Width | Thickness |
| Notation | (mm) | (mm) | (cm) | (mm) | (Mils) |
| 10 × 10 | 10 | 10 | 1.0-2.5 | 0.2-3 | 11-25 |
| (or a single | | | | | |
| ring system of | | | | | |
| this diameter) | | | | | |
| 15 × 15 | 15 | 15 | 1.0-2.5 | 0.2-3 | 11-25 |
| (or a single | | | | | |
| ring system of | | | | | |
| this diameter) | | | | | |
| 20 × 20 | 20 | 20 | 1.0-2.5 | 0.2-3 | 11-25 |
| (or a single | | | | | |
| ring system of | | | | | |
| this diameter) | | | | | |
| 25 × 25 | 25 | 25 | 1.0-2.5 | 0.2-3 | 11-25 |
| (or a single | | | | | |
| ring system of | | | | | |
| this diameter) | | | | | |
| 30 × 30 | 30 | 30 | 1.0-2.5 | 0.2-3 | 11-25 |
| (or a single | | | | | |
| ring system of | | | | | |
| this diameter) | | | | | |
| 35 × 35 | 35 | 35 | 1.0-2.5 | 0.2-3 | 11-25 |
| (or a single | | | | | |
| ring system of | | | | | |
| this diameter) | | | | | |
| 40 × 40 | 40 | 40 | 1.0-2.5 | 0.2-3 | 11-25 |
| (or a single | | | | | |
| ring system of | | | | | |
| this diameter) | | | | | |
| 45 × 45 | 45 | 45 | 1.0-2.5 | 0.2-3 | 11-25 |
| (or a single | | | | | |
| ring system of | | | | | |
| this diameter) | | | | | |
| 15 × 20 | 15 | 20 | 1.0-2.5 | 0.2-3 | 11-25 |
| 15 × 25 | 15 | 25 | 1.0-2.5 | 0.2-3 | 11-25 |
| 20 × 25 | 20 | 25 | 1.0-2.5 | 0.2-3 | 11-25 |
| 20 × 30 | 20 | 30 | 1.0-2.5 | 0.2-3 | 11-25 |
| 20 × 40 | 20 | 40 | 1.0-2.5 | 0.2-3 | 11-25 |
| 25 × 40 | 25 | 40 | 1.0-2.5 | 0.2-3 | 11-25 |

In essence, any size coil is contemplated from about 12 mm to 45 mm diameter. Suitable lengths of the deployed device are from about 0.25 cm to 3 cm. Shorter devices may also be employed, e.g., 1 cm in length, especially for placement in pulmonary veins having short trunks. Where devices are shorter, with less windings, e.g., 1-2 windings, e.g., 1.5 windings, the devices may be made more rigid, e.g., using a thicker ribbon. In some cases, shorter devices may be employed for single ring systems (though not exclusively). In general, for larger radius devices, thicker ribbons may be employed to allow for provide for substantially constant pressure to be exerted against a PV wall, where substantially constant means within about 25%, or within about 10%.

Ribbon widths may vary from about 0.25 to 4 mm, e.g., 0.75 to 1.5 mm (although in some cases curved ribbons and wires may also be used), and ribbon thicknesses (δ) may vary from about 11 mils to 25 mils, e.g., 11 mils, 14 mils, 17 mils, or the like. In some cases, even thicker ribbons may be employed, e.g., 60 mils. Overall lengths may be, e.g., 100 to 300 mm, e.g., 120 to 270 mm. Where rings differ in size, larger rings may be made thicker in order to regulate the applied pressure to a common value. In some implementations, it is been found that, for a 30 mm implant, a 17 mils thickness ribbon sufficient. But above 30 mm, thicker ribbons may be advantageously employed. Generally, ribbons are easier to deploy than thicker wires, and in addition thicker wire takes up more space in the vein.

As noted above, the coils may be configured in a symmetrical pattern, e.g., the diameter of the distal coil may be substantially equal to the diameter of the proximal coil. Alternatively, an asymmetric pattern may be employed having one end of the coil larger or smaller than the other end, e.g., a distal end may have a 15 mm diameter while the proximal end may have a larger 25 mm diameter. Using these values, the coils when undeployed may be significantly oversized compared to the vessels for which they are intended. They may be, e.g., oversized by 10-100%, e.g., 10-60%, e.g., 10-30%, and good results have been seen also for values of 45-55%, e.g., 50% oversizing. The following table illustrates exemplary devices which may be employed for particular vessels:

| VESSEL SIZE | DEVICE SIZE |
|---|---|
| (DIAMETER IN MILLIMETERS) | (DIAMETER IN MILLIMETERS) |
| 7-9 | 10 |
| 10-15 | 17-20 |
| 16-18 | 22 |
| 20 | 25-27 |

The size of windings within a particular ring may vary. For example, the diameter of each subsequent winding in a two-ring implant may decrease in a distal direction. In some implementations, a distal ring may employ windings having a common diameter, while the proximal ring may employ windings having a decreasing diameter (decreasing in a distal direction).

The rings may be designed to deliver a force against the tissue of between about 5 g/mm2 and 340 g/mm2, e.g., between about 20 g/mm2 and 200 g/mm2. The distal ring may provide a greater amount of force than the proximal one. Devices have been found efficacious which were configured to deliver a pressure of between about 0.01 to 0.20 N/mm2 in a cylinder or vessel sized from 10-25 mm, e.g., 0.05 to 0.20 N/mm2, although ranges of 0.04-1.4 N/mm2 have also been found therapeutic, e.g., 0.04-0.12 N/mm2. More specifically, for smaller diameters, pressures may be from about 0.07 to 0.20 N/mm̂2, for intermediate diameters, 0.03 to 0.05, and for larger diameters, 0.01 to 0.08. The overall force delivered to the vessel may be between about 1-9 N for a 15×15 device, 0.2-8 N for a 20×20 device, 0.3 to 7 N for a 25×25 device, 1-5 N for a 30×30 device, although it will be understood that these values may vary with the size of the device, including the thickness of the ribbon. Typical values found appropriate are from 0.2 to 10 N, in particular 0.3 to 6 N. In tests, implanting intermediate sized devices, e.g., 27 mm diameter devices, in a 19 mm vein, resulted in the vein extending to about 23 mm. Similar percentage increases are expected for other such devices.

It is believed that the amount of pressure necessary should be more than 10 grams per square millimeter, e.g., greater than 20 grams per square millimeter, but less than 340 grams per square millimeter, e.g., less than about 200 grams per square millimeter, as noted above. While it may be desired to have the ring(s) and helix or helices exert a relatively constant force around the circumference of the vein, it is more likely, given anatomical imperfections, that certain areas will receive more pressure than others. However, compliance of the ring and the use of the helix helps to distribute forces around the implant. In general it is believed that the amount of pressure needed will primarily be a function of the material used, the diameter of the artery or vein, and the thickness of the muscle sleeve. It is believed that if the radial pressure is too low, e.g., below the range noted above, the implant device will not provide the necessary pressure to isolate the vein. Moreover, if the radial pressure is too high, e.g., too far above the range, erosion of the vein may occur.

It is noted that the pressures disclosed above vary greatly from that of stents of similar sizes, in part because the force distribution is over a much wider area due at least in part to the ribbon cross-sectional shape of the implant device. Other distinctions are that the ends are smooth, not pointed, e.g., the ends are not pointed in a direction parallel to the axis of the rings. Such “pointiness” is characteristic of stents due to their method of confinement and deployment, e.g., via a balloon inflation. In this regard it is noted that the present PVID is not compressed like a stent is thus generally not capable of being expanded with a balloon. A further distinction is that the pressure applied at one portion generally becomes distributed dynamically along the ribbon. This is in contrast to stents, where pushing on one end results in translation or movement of the entire stent. FEA results indicate the importance of distributing force, and such distribution of force is easier to achieve with an asymmetrical device because the vessel generally tapers from the left atrium to the antrum to the os to the PV. In general it may be desirable to maintain the same amount of radial force, across different size implants.

In the same way, it is believed that the pressures and forces disclosed above and which are required to treat atrial fibrillation are higher than those seen in, e.g., endoluminal filters.

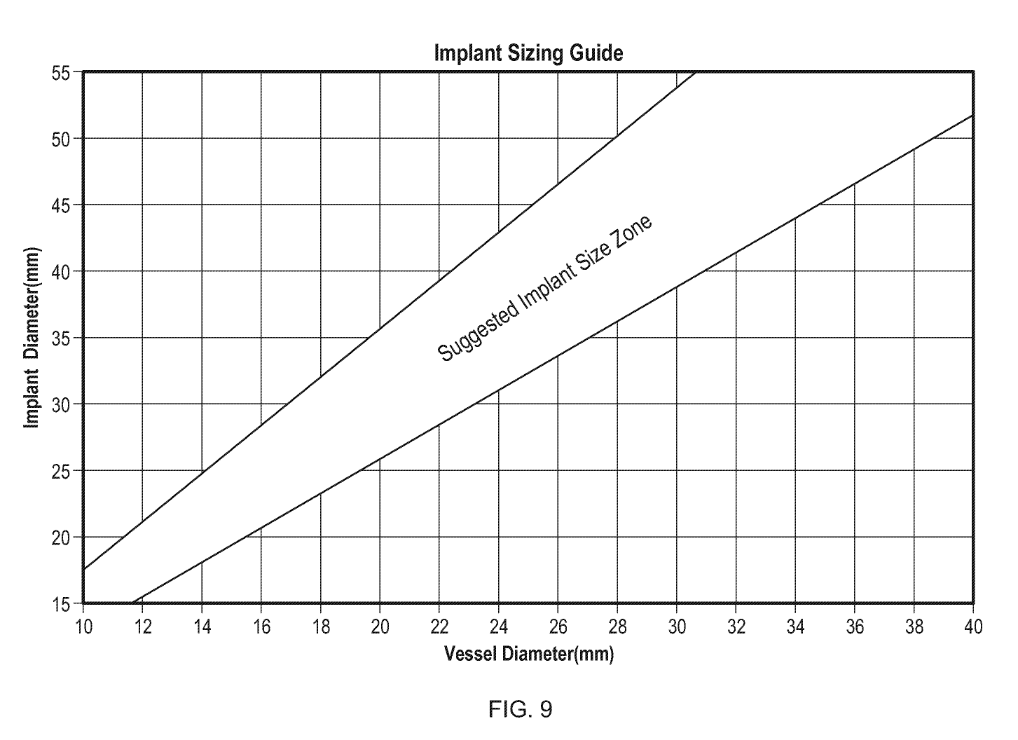

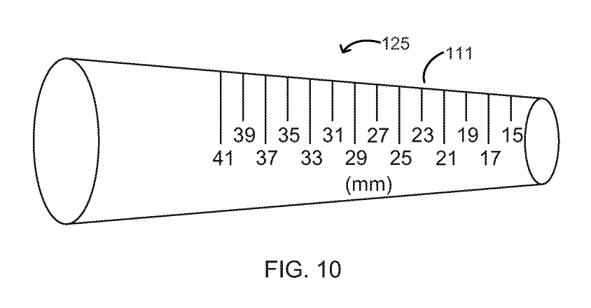

Suitable sizing has been determined and is illustrated by the Suggested Implant Size Zone of FIG. 9. In percutaneous implementations, the vessel sizing is generally determined by fluoroscopy, ICE, or the like. In surgical implementations, vessel sizing may be determined by a device such as the sizing device 125 of FIG. 10. In the device 125, gradations 111 (in mm) are illustrated on a conical-shaped tube, and by placing the tube in a vessel to be sized, as far as the tube can be inserted without distending the vessel, appropriate sizing can be determined.

One or more of the helices may revolve around a central axis less than 1, 1, 1.5, 2, 3, or more times. In this way, even when placed in larger veins, the available expansion room may cause an effective pressure block to be achieved. However, in this regard, it is noted that radial force may decrease dramatically as the radius increases.

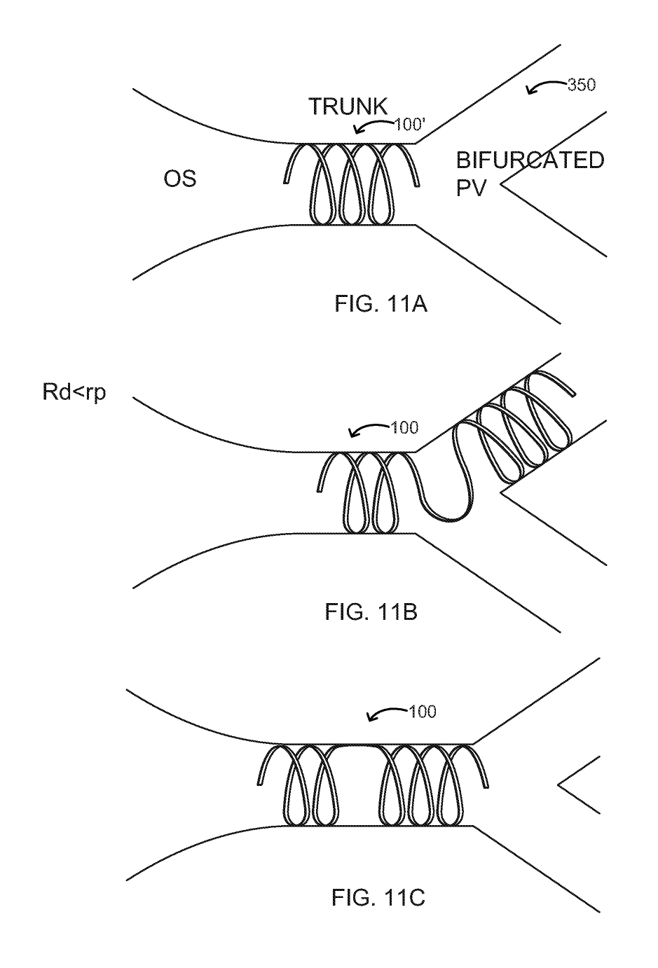

Referring to FIGS. 11A-11B, a single ring system 100′ or a dual ring system 100 (or other multiple ring systems) may also be employed in pulmonary veins which are bifurcated, i.e., have a common trunk which bifurcates to two separate pulmonary veins. In FIG. 11A, a single ring system 100′ is illustrated in the trunk of a bifurcated PV 350. It will be understood that the system 100′ may also be disposed in one of the bifurcations if desired by the physician and if practical to reach. In FIG. 11B, a dual ring system 100 is illustrated, with the proximal ring in the trunk and the distal ring in the bifurcation. In FIG. 11C, a dual ring system 100 is illustrated in the trunk.

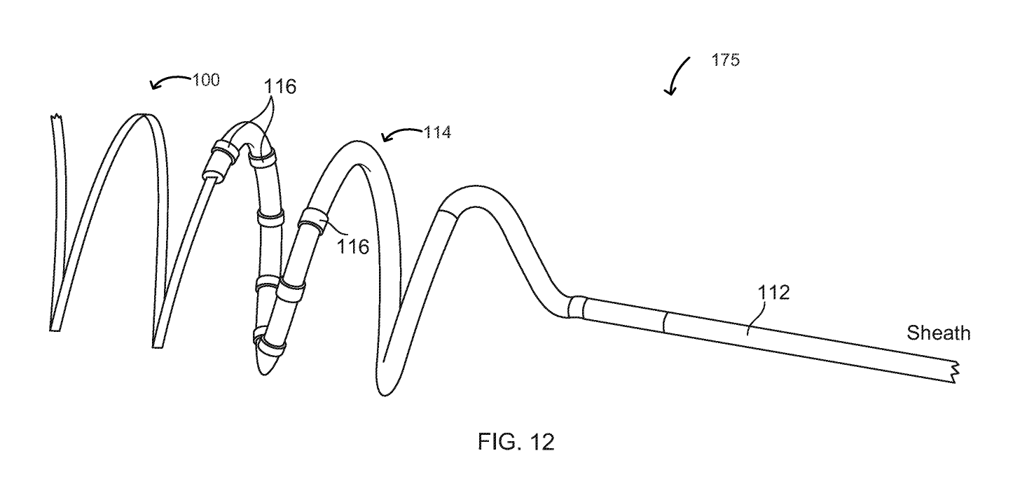

Referring to FIG. 12, a kit 175 is illustrated having a DSC 112 which couples to a pigtail distal end 114. The kit 175 further includes an implant 100, shown in FIG. 12 as partially extending from the DSC. The DSC 112 further includes electrodes 116 which may be employed for mapping as well as for delivering RF therapies. By having the electrodes 116 on the DSC 112, a determination of conduction in the pulmonary vein may be made both before and after implantation of the device 100. In addition, if implantation of the device does not result in complete block, the electrodes 116 may be employed to perform a supplementary therapy of RF ablation. Additional details of such DSCs are described below in connection with the embodiments of FIG. 41 et seq.

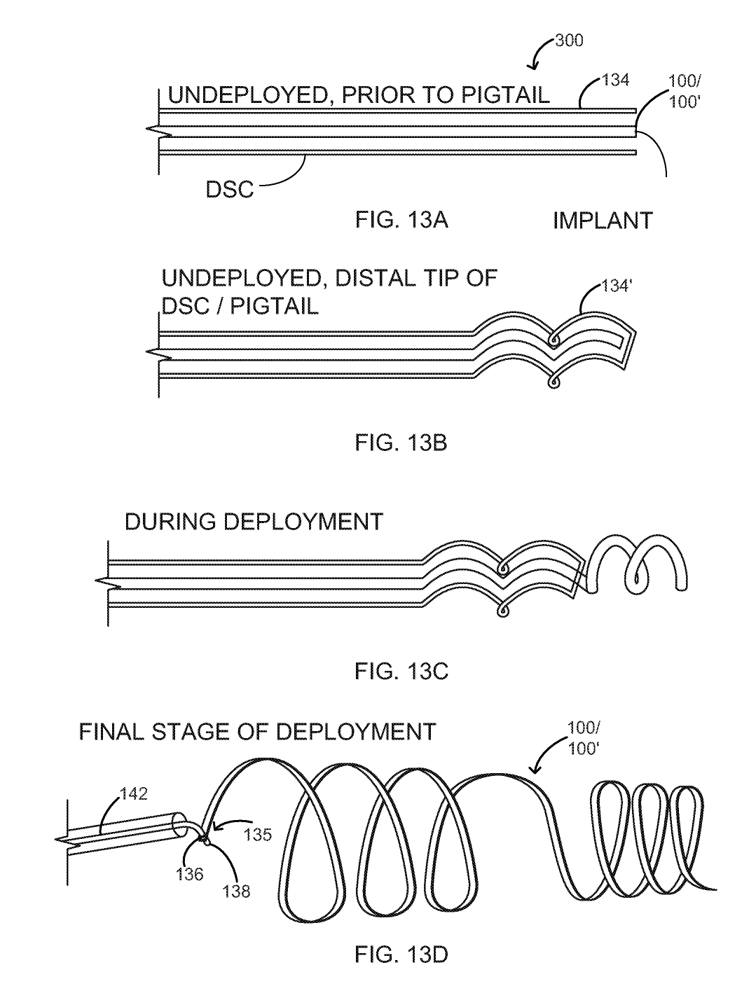

FIGS. 13A-13D illustrates stages and deployment of an implant 100 (or 100′) from a DSC 300. In particular, in FIG. 13A, a situation is shown in which the implant 100/100′ is undeployed, prior to a distal end 134 of the DSC being formed into a pigtail. In FIG. 13B, the distal end of the DSC 134 is formed into a pigtail 134′. In FIG. 13C, the implant 100/100′ is partially deployed. In FIGS. 13B and 13C, the distal end of the DSC is shown schematically so that the implant within may be more easily visualized, but it will be understood that the distal end of the implant generally may appear as in FIG. 12. In FIG. 13D, the implant 100/100′ is close to being fully deployed, being attached only at a point 135 to a central core 142. In this figure, a hook 138 engages a keyway 136 at the proximal end 135 of the implant 100/100′.

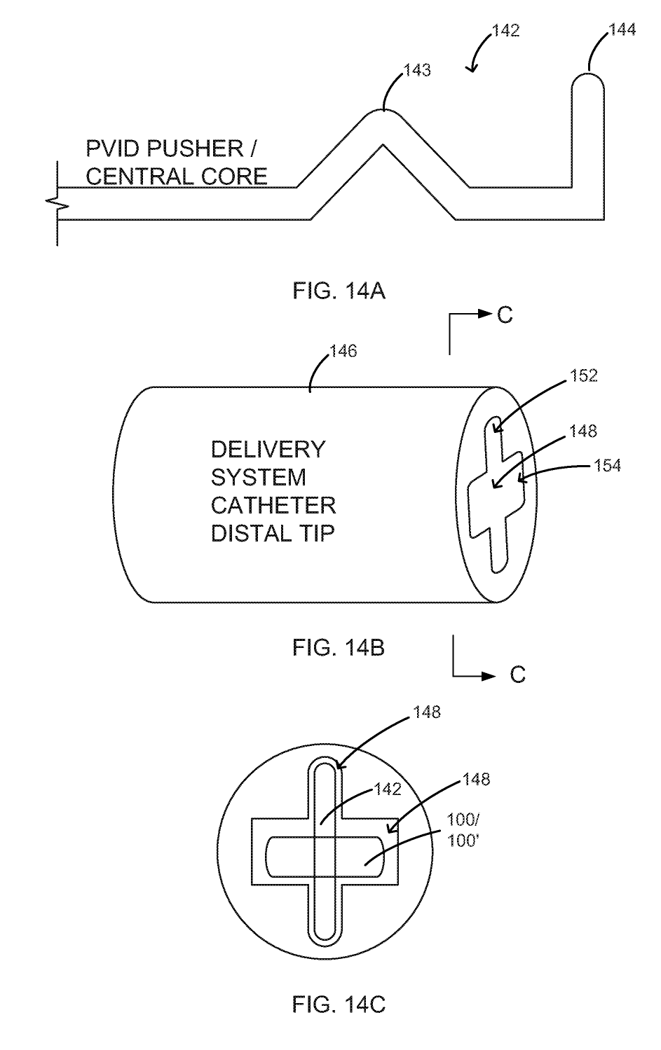

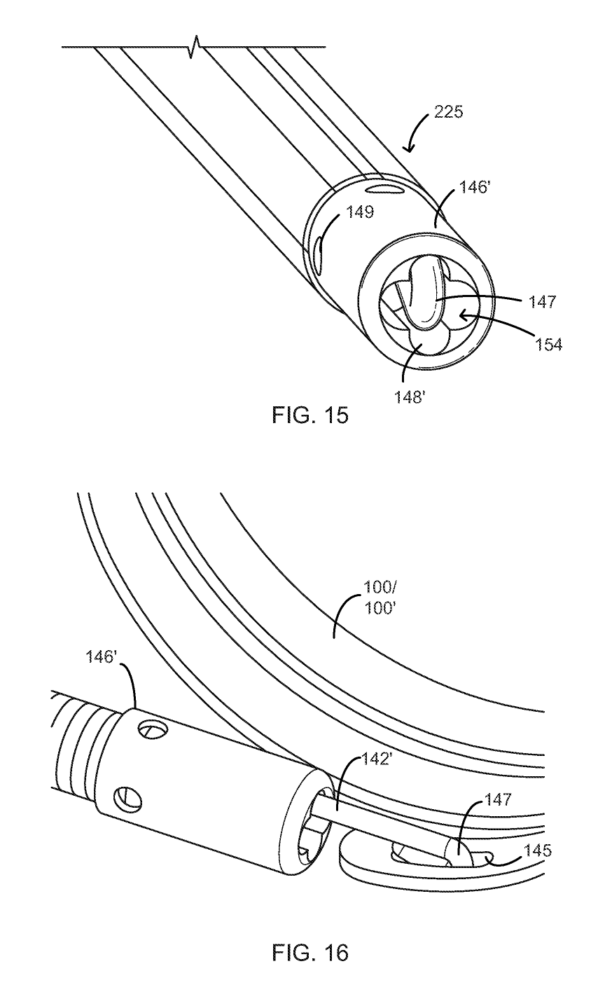

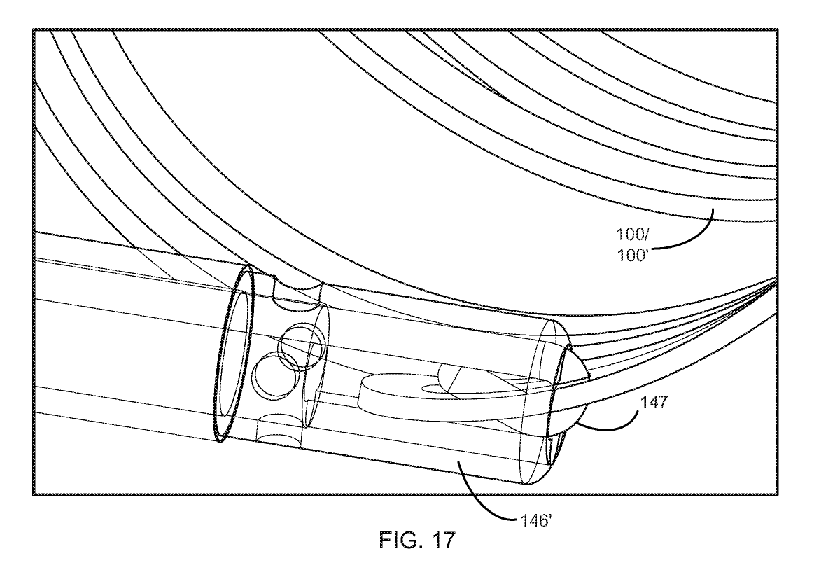

FIG. 14A-14C illustrates another exemplary central core and DSC implementation. In particular, a pusher or central core 142 for a PVID is illustrated having a hook or tab 144 for engaging a PVID. A notch 143 may be optionally disposed in the central core 142 such that, upon extending from a DSC, the notch 143 forces the tab 144 downward and out of engagement with a keyway (not shown) of a PVID.

FIG. 14B illustrates a distal tip 146 of a DSC which may be employed with the central core 142. The interior configuration of the distal tip 146 need not be employed throughout the length of the catheter, as illustrated, but merely at the distal tip. Of course, in an alternative implementation, the hole features disclosed below may be embodied along the length of the catheter.

In the implementation of FIG. 14, the distal tip 146 may form a cylindrical tip which is bonded (via the glue or weld ports 149) to the end of the catheter. The distal tip 146 may have defined therein a hole 148. The hole 148 may include a portion 152 intended to engage the tab 144 and a portion 154 is intended to engage and hold against relative rotation the implant device. FIG. 14C illustrates the situation in more detail, including a representation of the implant device 100/100′.

FIGS. 15 and 16 illustrate a similar distal tip 146′, e.g., a cross tip retainer, disposed at the distal end of a DSC 225. The cross tip retainer may be, e.g., 0.25-1.5 cm in length. As may be seen in FIG. 15, a central core, also termed a central core wire, includes a distal end 147. As may be seen in FIG. 16, the central core distal tip 147 (at the distal end of the central core 142′) engages a keyway 145 in the implant 100/100′. When the ribbon of the implant 100/100′ is within the hole 154′ defined in the distal tip 146′, and the distal end 147 is disposed in the keyway 145, the central core 142′ securely holds and can move the ribbon of the implant device 100/100′. In this way, manipulation of the central core 142′ by the physician allows the same to install the implant device 100/100′ at an arbitrary location, e.g., within a pulmonary vein of a patient. It is noted that the distal tip 147 of the central core 142′ may be constructed by merely bending a portion of the distal tip back upon itself. In such an implementation, the implant device 100/100′ is particularly easy to release upon successful installation of the device within a pulmonary vein. Typically, as will be described, successful installation is one in which a level of conduction measured post-implantation (the second value) is less than a level of conduction measured pre-implantation (the first value), e.g., by at least 50%. FIG. 17 illustrates interior details of the distal tip 146′ with the distal end 147 securely holding an implant device 100/100′ (at its keyway 145) therein.

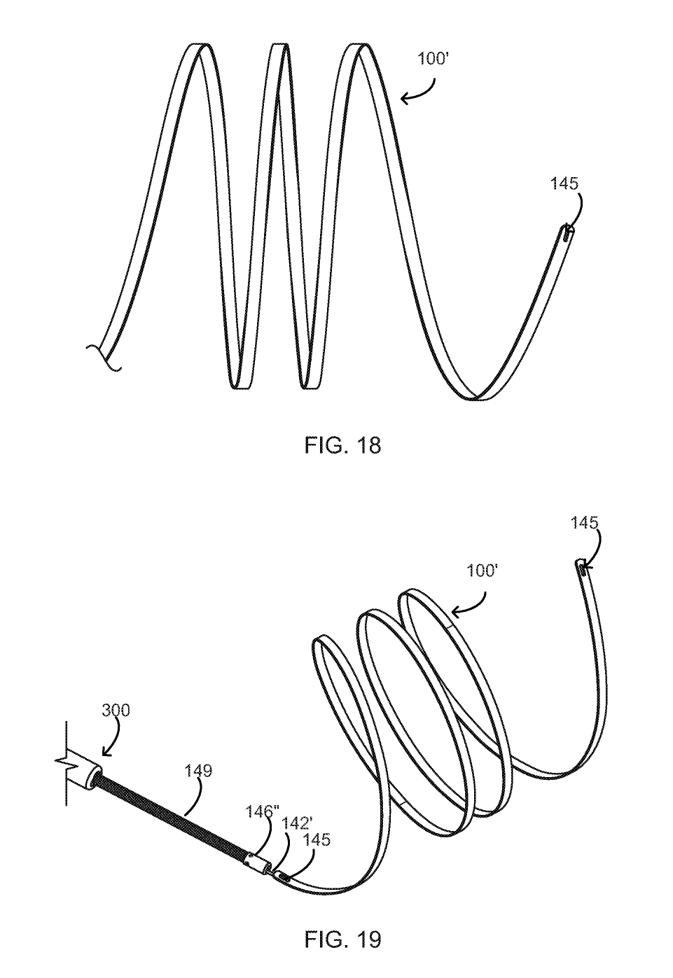

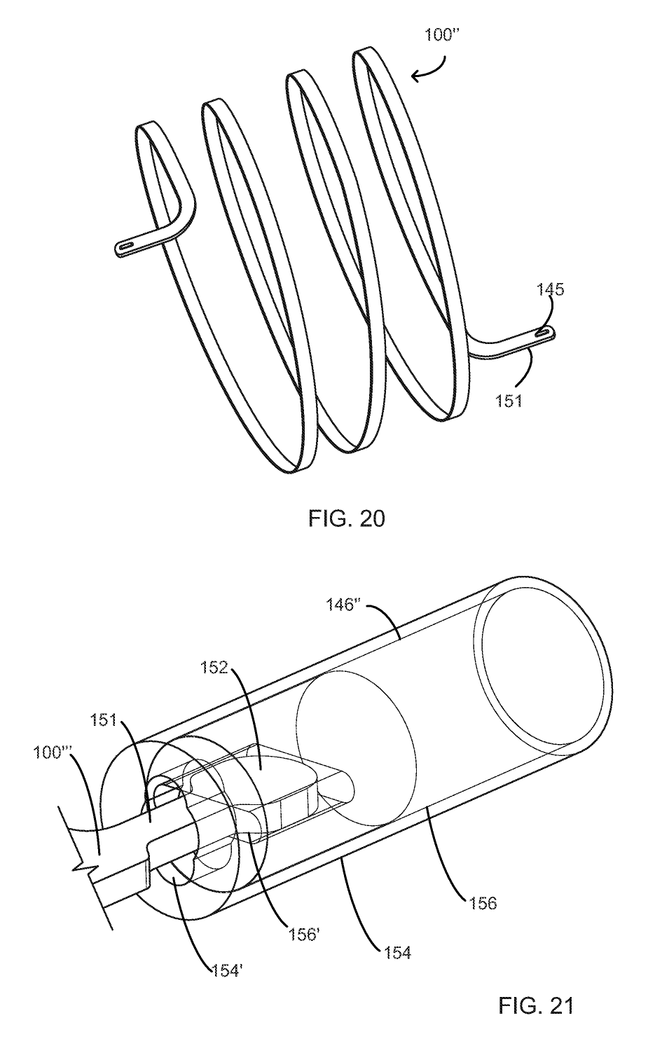

FIGS. 18 and 19 illustrate a single ribbon system 100′, i.e. a ribbon forming a helix having a single ring, the ring comprising more than one coil or winding. In these figures, the number of coils or windings is greater than three. Keyways 145 are shown on both the proximal and distal ends of the device 100′. In FIG. 20, the device 100′ is shown exiting a distal tip 146″ of a DSC 149, the DSC 149 emerging from a transeptal sheath 300. The device 100′ is coupled to the delivery system via the central core 142′, and in particular by engagement of a distal end (not shown) of the central core 142′ with the keyway 145 on the proximal end of the device 100′.

FIG. 20 illustrates an alternative implementation of an implant device, herein termed device 100″. The device 100″ includes a distal end 151 (and proximal end) with keyways 145, the ends are substantially perpendicular to the plane of the helical rings of the device 100″. For certain DSC's, such perpendicular ends allow for a more convenient connection of the implant device to the DSC.

FIG. 21 illustrates an alternative implementation of an implant device, herein termed 100′″. The device 100′″ includes a proximal end 152, which generally has a bulbous or other shape to maintain the same in locking engagement within an enclosure within the distal tip 146″. The proximal tip 152 is held in place within two cylindrical tubes 154 and 156, the cylindrical tube 154 defining a hole 154′ to an exterior of the same, and the cylindrical tube 156 defining a hole 156′ to an exterior of the same. The holes can rotate around a neck 151 of the implant but hold in place the proximal end 152. Only when the holes 154′ and 156′ are in alignment, can the proximal end 152 emerge from the distal tip 146″. Put another way, only when the holes 154′ and 156′ are in alignment, can the device 100′″ be released from the distal tip 146″. Generally, once the holes 154′ and 156′ are in alignment, the strain of the device 100′″, or a proximal movement of the distal tip 146″, will cause the release of the device 100′″ from the DSC. In essence, the holes 154′ and 156′ form a locking collar, and by twisting the cylindrical tubes 154 and 156 relative to each other, the locking collar can be made to unlock the implant.

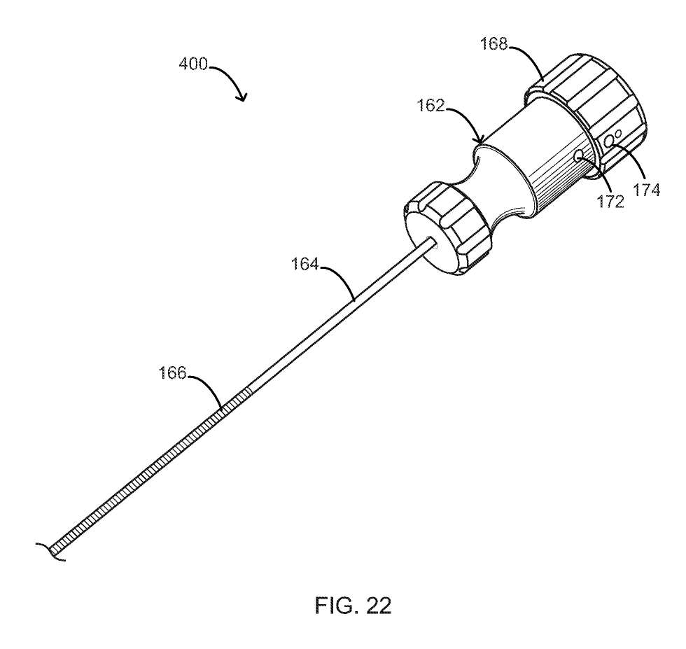

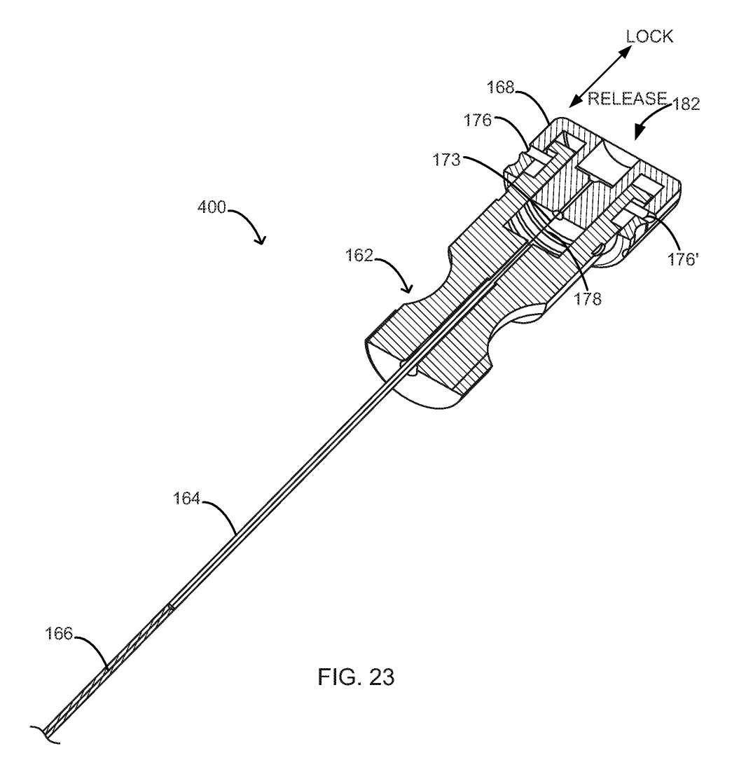

FIGS. 22 and 23 illustrate an exemplary implementation of a portion of a DSC, and in particular a deployment handle assembly 400 of the same. The handle assembly 400 includes a deployment handle 162 and a lock knob/release knob 168. The deployment handle 162 is coupled to a hypotube 164 which is in turn coupled to a flex shaft or coil 166. Alignment dots 172 and 174 are employed to visually demonstrate to the physician when the device is capable of being deployed and released into the patient. In various potential fashions, alignment of the dots indicates when actions can be taken or not taken with respect to the implant. For example, if the dots are aligned, a button may be depressed on the end of the lock knob/release knob (not shown) which permanently releases the implant, e.g., into the pulmonary vein of a patient, e.g., by forcing a distal end of a central core wire out of the DSC, thus allowing a proximal end of the PVID to move away from an engaging boss, deploying the final portion of the implant.

FIG. 23 illustrates the deployment handle assembly 400 in more detail. Various elements have been described with respect to FIG. 23 and their description is not repeated here. FIG. 23 also illustrates the core wire 173, and a tension spring 178 which provides pressure against the core wire plug 182. The guide pins 176 and 176′ guide the rotation of the core wire plug 182 relative to the handle 162, and when the appropriate alignment has been obtained, depression of the core wire plug 182 relative to the handle 162 allows the final release of the implant as transmitted by the core wire 173.

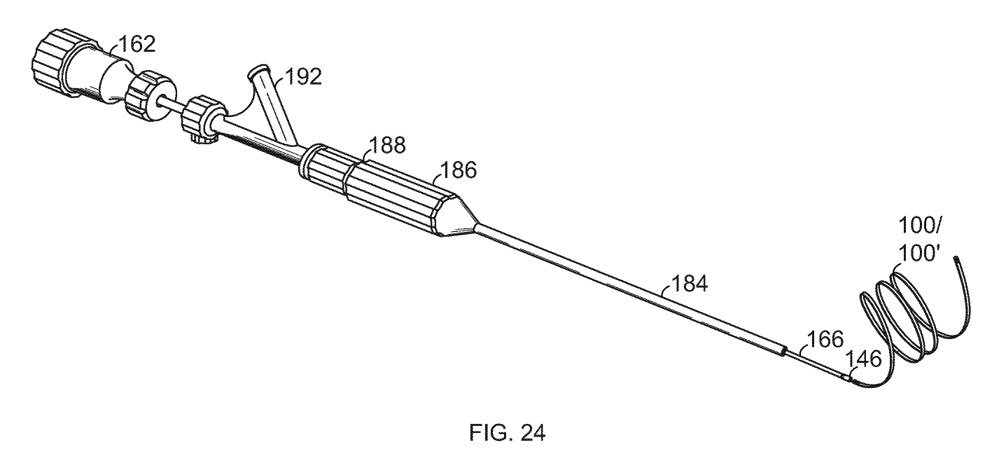

FIG. 24A illustrates the deployment of an implant device 100/100/from a handle 162. FIG. 24 further illustrates a hemostasis valve 192 with flush port and a torque handle 186 coupled to the hemostasis valve portion via a luer 188. A proximal shaft portion 184 is illustrated, along with a flexible shaft portion 166. A cross tip implant retainer 146 is illustrated, the same or similar elements seen in FIGS. 15-17, 19, and 21.

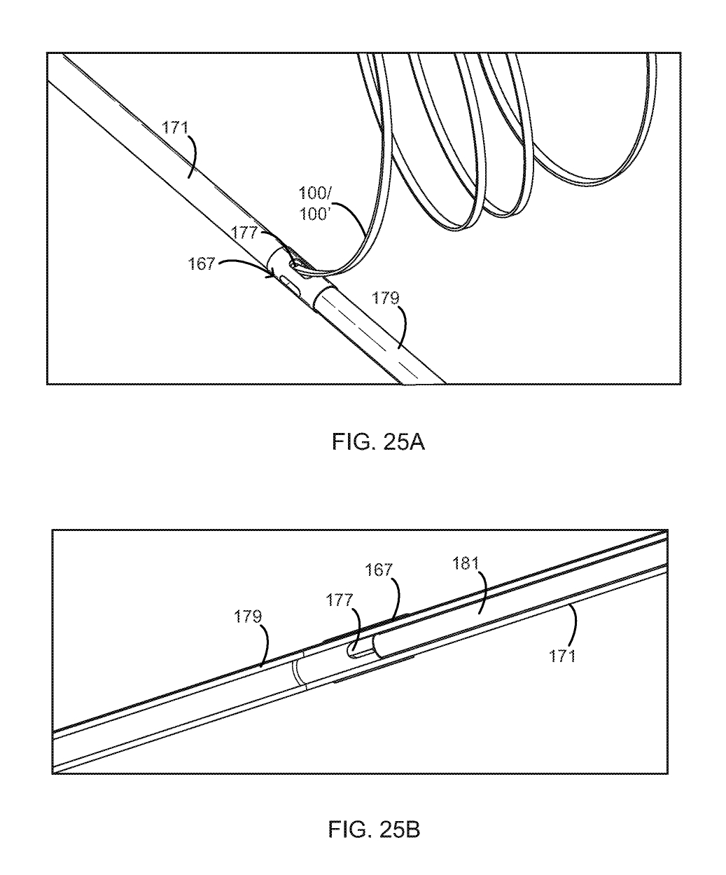

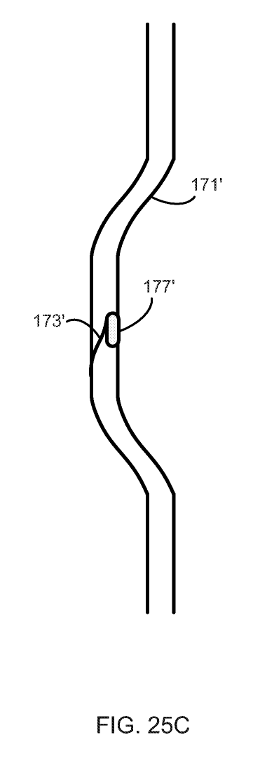

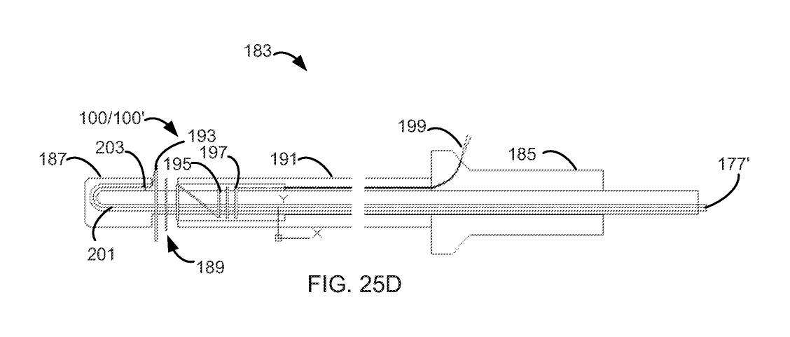

FIGS. 25A and 25B illustrate an alternative implementation of a DSC distal tip, having a side port assembly 167 through which the implant device 100/100′ emerges. The side port assembly 167 has at least one hole 177 defined therein. In the implementation illustrated in FIG. 25A, a quad port design is illustrated with four holes defined. The side port assembly 167 may be at the distal end of the DSC, or as illustrated, may have a proximal shaft 171 bonded or otherwise attached at a proximal end and a distal segment 179 attached at a distal end. And a distal and of the distal segment 179 may be an atraumatic tip. A guide wire lumen may extend from the atraumatic tip back through the handle.

Referring in addition to FIG. 25B, a polymer, e.g., polyimide, sleeve 181 may line the inner wall of the proximal shaft 171. The sleeve 181 provides that the implant will not be blocked by any defects or imperfections of the inner wall of the proximal shaft. The sleeve 181 may extend at least somewhat into (and thus covering) the holes 177.

Due to the curve of the implant, once the distal end of the implant is extended to the holes 177, the implant will generally exit the nearest hole. Such may be assisted by the shape of the inner wall of the side port assembly 167 between the holes. For example, a triangular or wedge-shape or the like may be defined by the portions between the holes, forcing the implant into one or another of the holes 177 and thus deploying the implant. A ramp may also be provided for this purpose, forcing the implant ribbon out of the lumen, although in many cases the natural curve of the implant (due to its set helical shape) will force the same out of the lumen and into a deployed configuration.

FIG. 25C illustrates an alternative implementation of a shaft 171′, the shaft employing a double bend within, a portion of the shaft between the bends defining an exit hole 177′. Due to the double bend, the portion of the shaft between the bends naturally adopts a position adjacent the vessel wall. By placing the exit hole in this portion, when the implant device exits the catheter, it is forced to exit in a direction away from the vessel wall, reducing the risk of perforation. The implant can be forced to exit through the hole 177′ using one or more ramps on the interior of the shaft, an exemplary one of which being illustrated as 173′.

FIG. 25D illustrates an alternative implementation of a DSC 183, the DSC 183 including a handle 185 and a distal end 187. A catheter shaft 191 of the DSC is split, forming a hole 189 through which an implant 100/100′ may be deployed. The implant 100/100′ is illustrated, with an exemplary coil being deployed 193, and an exemplary coil 195 undeployed. In many implementations coil 195 is not in a coil shape when in a catheter lumen, but is in a straightened shape. A first central core wire 199 is attached to the implant 100/100′ at a point 197, while a second central core wire 201 is attached to the implant 100/100′ at a point 203. Each core wire may be coupled to a deployment device as illustrated in the device 400 of FIG. 23, such that a momentary depression of a button may force the distal ends of the core wires out of the DSC and thus release an end of the implant attached thereto. In many cases such control of both ends of the implant may be advantageous and allow precise control of the positioning of the PVID implant.

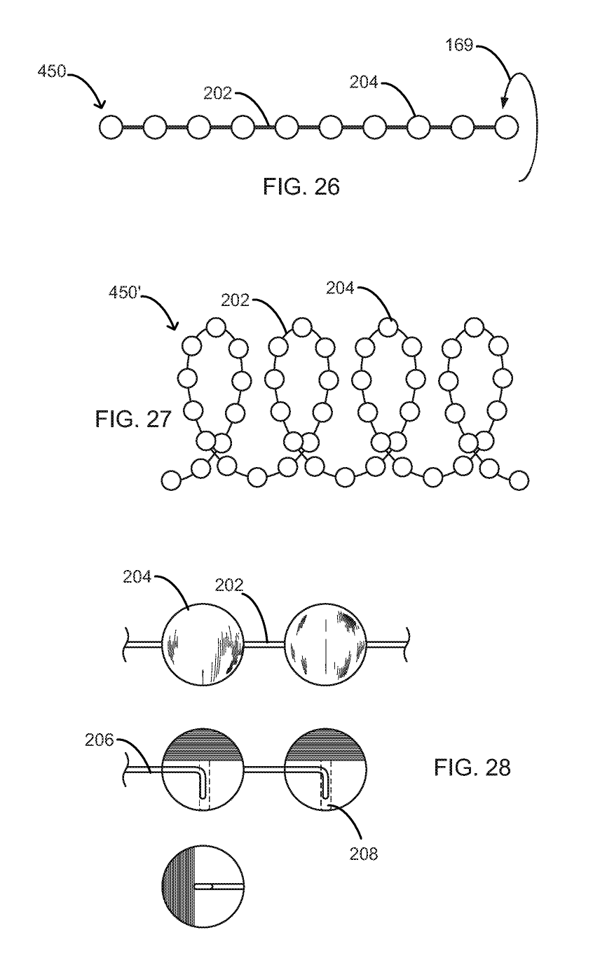

FIGS. 26-28 illustrate alternative implementation of the implant device, with reference numeral 450. In this implementation, a series of balls 204 are connected via links 202. The balls and links may be nitinol or another type of biocompatible material. Due to the linear nature of the system, the same may be deployed using delivery catheters of the type illustrated elsewhere in this specification. The DSC may temporarily hold one ball, e.g., a proximal ball, and by rotating the ball in a direction, e.g., shown by arrow 169, the system may take the shape shown in configuration 450′. The implant maintains configuration 450′ because of a locking mechanism illustrated in FIG. 28. In particular, the end of the ball is rotated until all of a set of locking arms 206 are secure within respective slots 208. The locking arms 206 may become secure within the slots 208 in a number of ways, e.g., by virtue of a friction fit. The implant size depends on the length of the links between the balls and the angle of the locking arm.

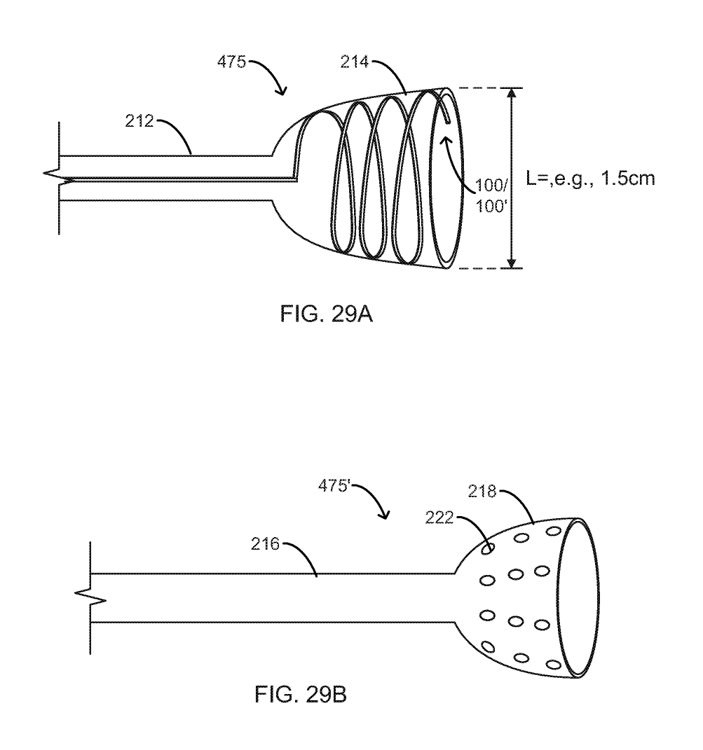

In another implementation, FIGS. 29A and 29B illustrate alternative implementations of the DSC 475. The DSC 475 includes a distal shaft 212 coupled to an umbrella or cup shaped distal section 214. As the implant 100/100′ traverses from the distal shaft 212 to the cup shaped distal section 214, it expands to the extent allowed by the section 214. Upon traversing further, e.g., by retraction of the DSC, by maintaining the implant in a stationary position, the implant is deployed. The distal section 214 may be collapsed in known manner and may take its shape using polymer heat setting, inset spines, via balloon inflation, or the same may be formed and maintained in that configuration, then collapsed into the DSC during installation in a patient. Post-implantation, the same may be retracted into a DSC lumen or the lumen of a transseptal sheath.

FIG. 29B illustrates an alternative implementation, where a DSC 475′ has a shaft 216 and a distal section 218. The distal section 218 includes a number of electrodes 222, which may be employed for pacing, ablation, or the like.

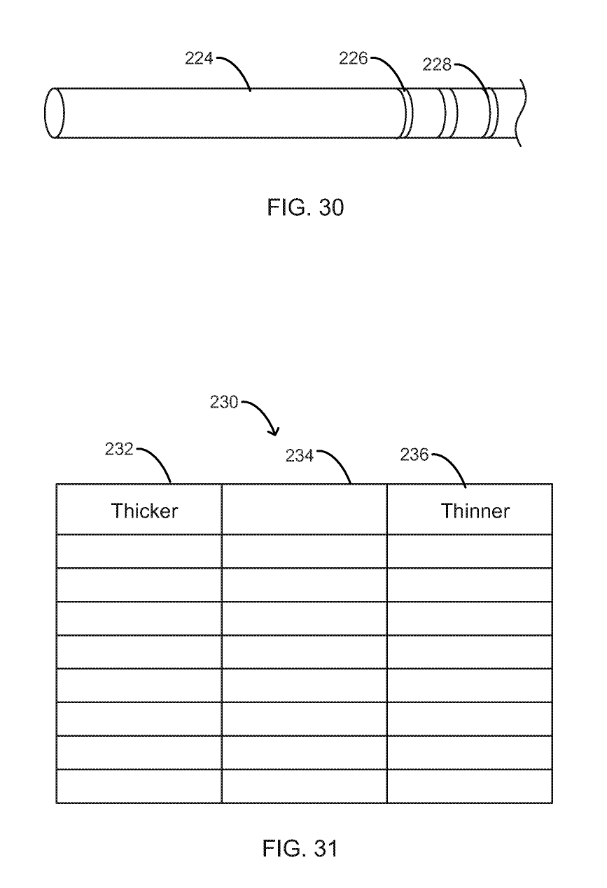

Referring to FIG. 30, an implementation of a DSC distal portion 224 is illustrated. Marker bands 226 and 228 are illustrated, and the same may be disposed on the DSC or on the PVID or even on the central core wire. Such marker bands are generally radiopaque, and allow convenient visualization of the distal portion of the DSC or PVID such that the same may be maneuvered into a desired location, e.g., the PV. Not only the location but also the shape of the appearance of the marker bands may provide useful information. For example, if the marker bands are on the DSC or on the PVID and appear oval instead of circular, it can be inferred that the direction of viewing is off-axis, and adjustments can then be made if warranted. Marker bands may also be employed to determine if the PVID has been correctly deployed versus being improperly deployed because of an irregularity within the vessel.

Referring to FIG. 31, a sheet 230 is illustrated which may be employed in the manufacture of an implant such as the PVID. In particular, the sheet is generally a planar sheet of biocompatible material which is cut into strips to form the ribbon from which is formed the PVID. The ribbon may then be treated to be so formed. For example, where the material is nitinol, the nitinol may be cold-worked or heat-set to configure the same into a ring or helical shape. In one implementation, the sheet has a common thickness throughout. In another implementation, as shown in FIG. 31, one section 232 is thicker than a middle section 234, which is in turn thicker than a section 236. The thinner sections may be formed into rings having smaller diameters, and the thicker sections may be formed into rings having larger diameters. In this way, the pressure caused against the vessel is more equalized between the smaller diameter rings and the larger diameter ring. For example, the pressure may be substantially the same to within about +/−25%. The way in which a section may be made thinner can vary, e.g., via bead blasting, chemical etching, or the like.

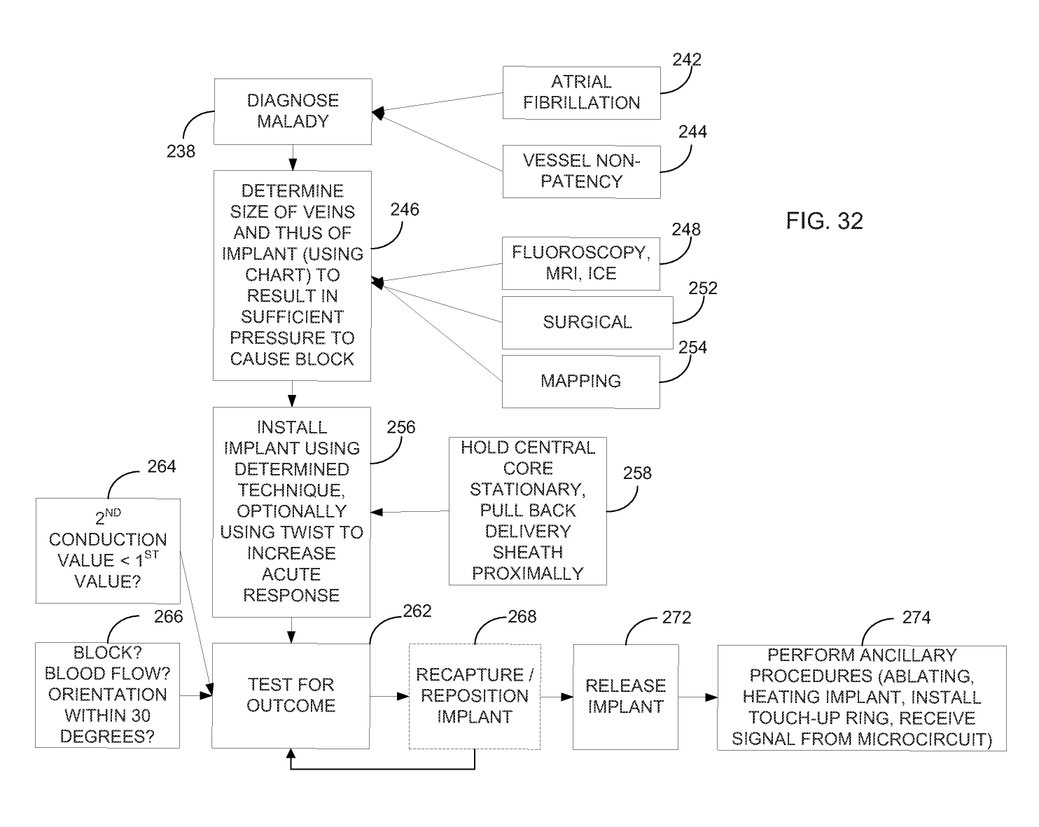

Referring to FIG. 32, a flowchart is shown detailing an exemplary implementation of a method of the invention. In a first step, a malady is diagnosed (step 238). The malady may be, e.g., atrial fibrillation (step 242), vessel non-patency (step 244), or the like.

As the device relies to a certain extent on pressure applied to a vessel, and the pressure is to some extent dependent on the geometry of the implant and the geometry and other characteristics of the vessel, a next step is to determine the size of the vessel, e.g., pulmonary vein, and thus determine the size of the implant necessary to result in sufficient pressure to isolate the vessel, e.g., cause conduction block (step 246). For example, the chart disclosed above in connection with FIG. 9 may be employed. The vessel size may be determined in a number of ways, e.g., using fluoroscopy, MRI, or ICE (step 248); by direct measurement during a surgery (step 252); or using another form of mapping as may be known or may be developed (step 254).

The implant may then be installed (step 256). The implant may be installed using the delivery devices and techniques disclosed above. A twist may be employed to increase the acute response. For example, just before releasing the implant, the DSC and in particular the central core may be twisted in a direction to increase the diameter of the implant beyond what it would be in the absence of the twist. In this way, the acute response may be enhanced. While the implant may be pushed out of the DSC, in many cases it may be desirable to hold the implant stationary, i.e., hold the central core stationary, and pull back the sheath covering the implant in a proximal direction. In this way, the implant is deployed in a more controllable fashion, reducing the risk of perforation.

In some cases, especially where the implant is deployed from a location proximal of the distal tip of the DSC, the risk of perforation may be already minimized, and hence the implant may be deployed by being pushed out rather than being deployed by simply being uncovered or unsheathed.

Because of the presence of an acute response, the outcome of the procedure may be tested (step 262). For example, a first or initial conduction value may be measured, and a second conduction value post-implantation may be measured. If the second conduction value is significantly less than the first, e.g., by 50%, successful positioning and implantation may be presumed (step 264). Other markers may also be employed to test the outcome (step 266). For example, for use of the device to maintain patency, blood flow may be checked and used as a determinant for successful positioning, e.g., increased blood flow implies proper positioning. In yet another way, techniques such as fluoroscopy may be employed to check the orientation of the implant. If the orientation is within 10°-30° of the ideal, where the axis of the ring system is parallel to the axis of the vessel, again proper orientation may be presumed.

If the test of the outcome results in a determination of improper placement, the implant may be repositioned (if still attached to the central core) or recaptured (if release has already occurred) (step 268). Recapture may be by way of known snare devices. The testing step 262 may be repeated and if successful the implant may be released in the desired location (step 272).

Ancillary procedures may then be performed (step 274). Such may include ablating, using inductive or RF heating to heat the implant, installation of touchup rings, receiving a signal from a microcircuit on the implant if one is present, or a combination of these. For example, a physician may determine that the implant is properly placed but does not provide enough PV isolation. In this case, a touchup ring, e.g., one with just a single set of coils, may provide additional block. Ablation steps may also be performed to enhance the therapeutic effect. The ablation steps may take advantage of electrodes on the DSC or may employ a separate ablation catheter, e.g., for cryoablation or RF ablation. Induction may also be employed for charging or powering the implant as well as for heating.

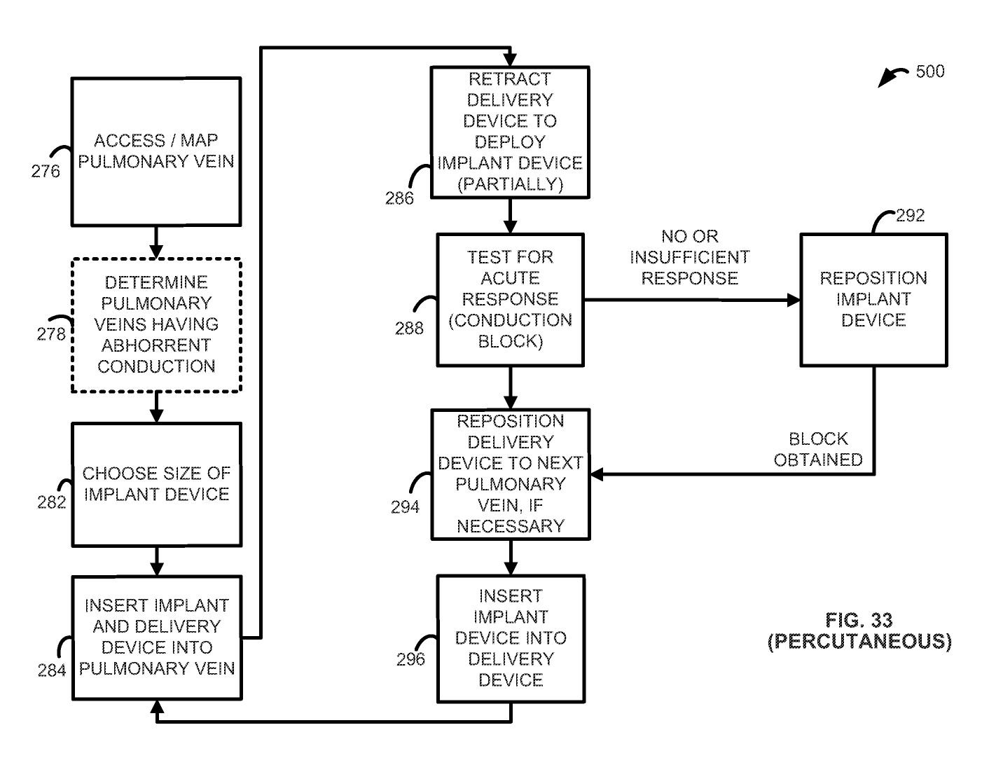

Referring to FIG. 33, a flowchart 500 having some similarities to FIG. 32 is shown, but the former being specified to percutaneous treatment of atrial fibrillation. A first step is that access and mapping are made of a pulmonary vein (step 276). Such generally involves a transseptal puncture, and oftentimes fluoroscopy or other techniques are used to enable the physician some degree of visualization of the cardiac system. A next step, which is optional, is to determine which pulmonary veins are susceptible to abhorrent conduction conditions (step 278). In most cases, all pulmonary veins will be assumed to contribute to the patient's atrial fibrillation. Based on the size of the veins, a size of implant device may be determined (step 282). Such may employ, e.g., the chart of FIG. 9. Following this the implant may be inserted and delivered into the pulmonary vein (step 284). In so doing, the delivery device may be extracted to deploy the implant at least partially (step 286). An acute conduction block response may be tested for (step 288), and if necessary the delivery device may be employed to reposition the implant device (step 292). Once sufficient block is obtained, the delivery device may be repositioned to the next pulmonary vein (step 294). The implant device may be coupled to a central core and inserted into the delivery device (step 296). The implant may then be delivered to the pulmonary vein (step 284), and the steps may be repeated until all pulmonary veins are treated.

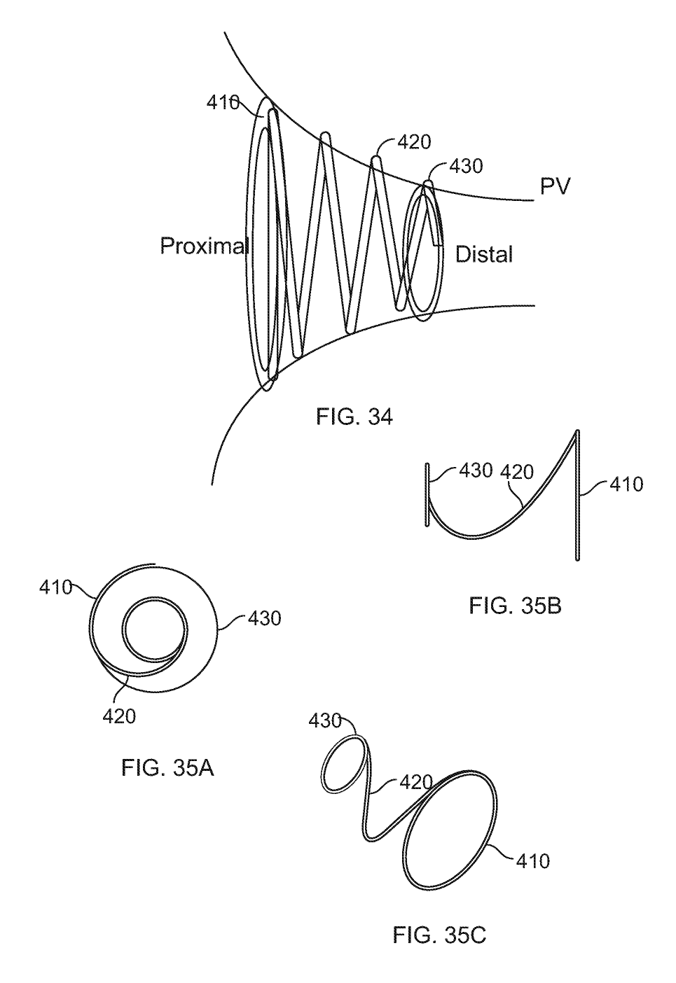

Referring to FIG. 34, an exemplary dual ring system for an implant device or PVID is illustrated having a proximal ring 410, a distal ring 430, and a helical extension arm 420 extending between the two. The device is illustrated in a pulmonary vein. FIGS. 35A-35C illustrate various views of the system of FIG. 34. FIGS. 36A-36C illustrates a situation in which dual helical arms 420′ extend between the rings 410 and 430.

To ensure a minimum of migration, the ends of the wire or ribbon forming the ring system may be scalloped or have another shape to increase frictional or mechanical resistance against movement. Such shapes are illustrated in FIGS. 37 (A)-(B). In FIG. 37 (A), a distal end 424 includes scallops or ribs 426, while in FIG. 37 (B) distal end 428 includes smaller but more frequent scallops or ribs 432. In addition, the external surface of the implant may have a textured surface, or may include a polymer sleeve, or a combination of the two, to further aid the device in fixation of the vessel. The polymer sleeve may include a Dacron coating, PTFE, or ePTFE, and other such polymers. The polymer sleeve may also include a microcircuit 429 to wirelessly transmit signals indicative of conduction during and/or after the procedure. Additional details of such a microcircuit are disclosed in greater detail above and below. Furthermore, a coating or biological agent of the implant surface may be employed to further reduce migration and/or erosion of the implant.

Optional holes 427 may be employed to assist in the process of endothelial cell formation.

Besides being placed on the polymer sleeve, a circuit 429 may be provided on the tissue side of the implant to perform mapping and/or optional pacing functions.

Referring to FIG. 39, a distal end 434 may further include a club shape 436 so as to minimize the chance of perforation. It will be understood by one of ordinary skill in the art that the club shape may be replaced with a ball-shaped end or the like to promote non-perforation.

Also referring to FIG. 39, the hole in the club-shaped end may be employed to allow two implants to be attached to each other. In this way, multiple implants may be loaded into a delivery system to allow multiple installations in a single procedure. The implants may be attached end-to-end in a way akin to staples or railcars.

The ring may employ a shoulder 418 for stability, as well as a feature 422 to cause pressure, as illustrated in FIG. 38. Such a feature may help with generating deep fibrosis in a vessel, thus assisting the creation of nonconductive tissue. For example, the feature 422 to cause pressure may be any three-dimensional solid capable of exerting additional pressure along a predetermined area, such as a ridge. The portion of the shoulder adjacent to tissue may be roughened or otherwise treated in order to provide an irritant to that tissue, so as to cause endothelialization as discussed above. Such endothelial cells are typically not conductive, and thus act as a long-term-care modality.

It is noted that limiting migration is assisted by the shape and structure of the implant device. In particular, the overall helical structure of the implant device ensures that a longitudinal force, along the axis of the device, tends to be absorbed by a compression of the helix, similar to the way in which a spring compresses, although the construction ensures that the spring constant may be extremely low, especially in the axial direction. This may be contrasted with other more stent-like structures, which are designed such that a longitudinal force is transmitted along the typical chain link or honeycomb structure, causing translation or a change of radius of such structures rather than compression.

Implant Variations

Other implementations of the implant device may include one or more of the following. The device may include a contiguous circumferential ring substantially normally perpendicular to the ostium of the PV, and the ring or coil structure may have at least 1 full rotation, as well as a pitch that is >1° from the first coil. The extension arms that join the distal and proximal rings may be designed to interrupt ectopic electrical signals emanating from within the PV. The ring or coil may have various cross-sectional shapes designed to focus mechanical force in a circumferential or helical pattern against the inner surface of a vessel or structure within the heart. These shapes include but are not limited to round or circular, triangular, rectangular, “U”-shaped, or any number of other shape combinations. The ring or coil structure may have a hexagonal, pentagonal, and/or octagonal shape when viewing in an end view. This geometric shape may be designed to improve conformability to the vessel following implantation. The ring or coil may have a material composition and/or geometry designed to sufficiently conform to tissue to prevent coagulation or thrombus, and may include a material coating to further reduce or prevent such coagulation or thrombus.

In some implementations, the ring and helices may act as an electrical wave reflector, changing the course of the electrical wave back to its origin and in some implementations acting as a cancellation medium to electrical waves emanating from the source.

It is also noted that approximately 30% of PV's have an oval shape. By changing the geometry of the loop or ring, the ring and vessel may be mutually conformed, and the radial force equalized along the circumference of the inner surface of the PVs. The ring or coil may have the above-noted shapes at the proximal end but may employ a circular shape at the distal end. The implantable devices may be employed in combination with an ICD to deliver currents or voltages to heart tissues. Such devices may be coupled to an ICD in a wired fashion or wirelessly. Other devices that may take advantage of the convenient placement of the implanted devices may similarly benefit from coupling to the same.

In another alternative implementation, the rings may be discrete and can even be discontinuous, in which case the same may be connected together by a long spine and expanded by a balloon. The rings, and in particular the coils thereof, may in some cases not form complete circles.

Delivery and Deployment

The device may be deployed in various ways. In general, the PVID is transported in a straightened (and undeployed) configuration using the DSC. Depending upon implementation, a distal tip of the DSC may remain substantially straight or may adopt a pigtail shape. In any case, once deployed, the PVID emerges with its axis parallel to the catheter and takes on the shape of the ring(s) and extension arm. Generally, due to the super elasticity and shape memory character of the PVID, the same not only takes on the desired shape but also may self orient within the vessel in various ways. The DSC may be, e.g., 9-12 French. Even smaller DSCs may work, e.g., 7 French, but the same occasionally encounter disadvantages in that their flexibility causes the same to adopt the shape of the indwelling implant, and thus acquire a bend or curve. A steering capability may be provided, e.g., bidirectional or unidirectional steering, although steering is generally not required.

One advantageous method is to deploy enough of the PVID to obtain purchase in the affected vessel. For example, 1 to 1.5 turns may be deployed. Following such partial deployment, the remainder of the PVID generally deploys in a rapid and highly accurate manner. Generally such deployment is not by pushing the implant out of the DSC, but rather by holding the implant stationary (by holding the central core) and retracting the DSC. In any case it is generally important to not advance the central core too far outside the DSC until optimal placement location has been confirmed. It is noted that the above considerations apply to both single ring and dual (or more) ring systems. It is also noted that one technique found useful is to deploy a portion of the PVID into the vessel, and then pull or push the same to situate the portion into a desirable location of the PV and os to provide block.

Good results have been found where the proximal ring is adjacent the os of the pulmonary vein and the distal ring is within the pulmonary vein, e.g., 2-4 cm within, as some reports have described the closest activation atrial fibrillation triggers to be about 2-4 centimeters within the pulmonary vein.

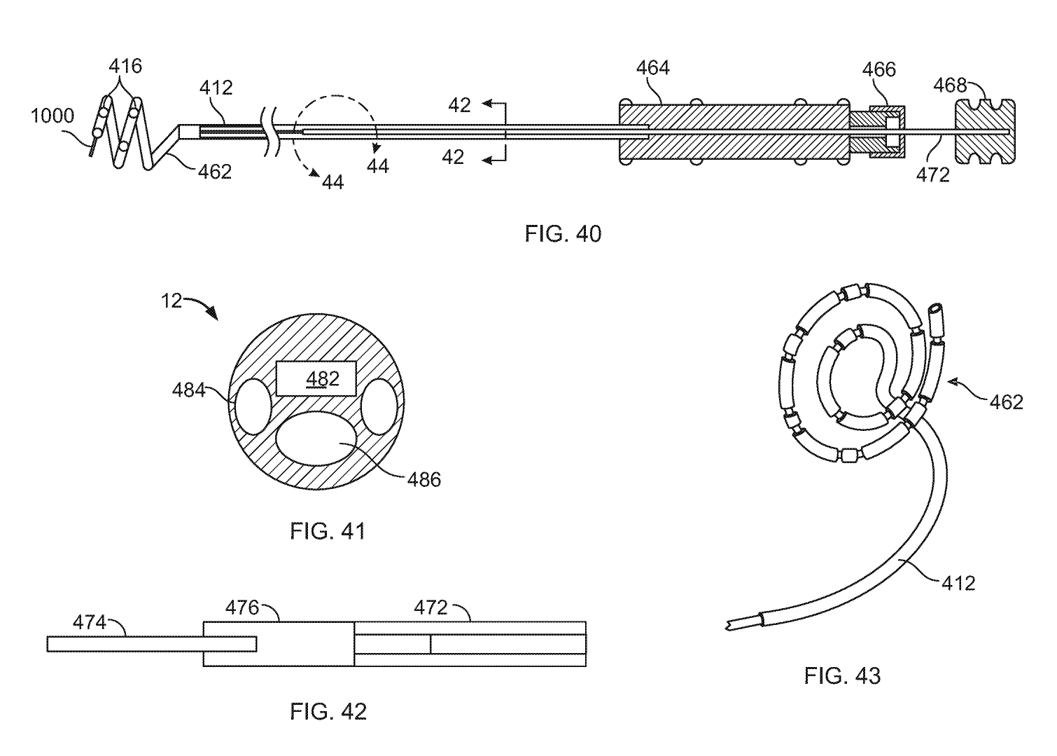

In one implementation, illustrated in FIGS. 40-43, a delivery catheter has a handle 464 for steerability and a knob 468 to control a pusher (or grabber or pushing means) 472, e.g., a flexible wire or elongated spring, at a proximal end. At a distal end, the delivery catheter may be straight or may have a PeBax® (or other material) loop or pigtail end. In general, it may be preferable for the pigtail to be substantially perpendicular to the longitudinal axis of the delivery catheter, e.g., within +/−25% or 10%. The pusher (shown in greater detail in FIG. 9) with a tip 476 extends through the delivery catheter 412, and the same is attached to an implant device 1000 at a point within the catheter. The implant device is uncoiled in this undeployed configuration, and the implant device may extend through the pigtail 462 and may further extend a short distance from the distal end of the pigtail during deployment. The distal end of the delivery system may also include a design where the catheter distal end is in a straight or neutral position and then steered using knobs and/or levers on the handle to create the pig tail distal segment. Another lever located on the handle may be employed to deflect or steer the distal segment for cannulation of each pulmonary vein. The distal end of the delivery system may also be straight, and a natural tendency of the implant to achieve a perpendicular orientation relative to the axis of the pulmonary vein may be employed to assure proper disposition and orientation within the pulmonary vein. This design may also include a plurality of electrodes 416 to enable intra-cardiac electrogram interpretation.

By deploying the implant device from of the distal end of the catheter, shown in more detail below, the same may take up a position within the PV as desired. One purpose of the PeBax pigtail is to protect the vein during deployment in the same way, e.g., a Lasso® catheter does. In addition, the PeBax pigtail may be equipped with electrodes to allow mapping and/or ablation, as described in greater detail below. The pitch of the distal loop or pigtail may be altered in known manner, e.g., by a control wire, to allow different cardiac geometries to be accommodated. Where mapping electrodes are used, their length may range, e.g., from approximately 0.5-4.0 mm. While the pigtail distal tip is generally at a distal end of the delivery catheter, the same may also be disposed proximal to the distal tip. The distal tip may have a maximum radial size of, e.g., 15 mm, 25 mm, or other radii as dictated by the anatomy. Using the pigtail, deployment of the implant in a vessel leads to an axis of the implant being substantially parallel to an axis of the vessel, where substantially parallel means between about 0° and 30°.

While the term “pushing the implant out of the distal end” above may refer to pushing the implant in a distal direction, the same is also used to refer to the situation where the absolute position of the implant stays constant, and the delivery device is moved in a proximal direction, thereby uncovering or revealing the implant and allowing the same to spring to a deployed orientation against the pulmonary vein wall.

“TWIST” technique

It is also been found that additional pressure against the vessel, and thus a more efficacious treatment of atrial fibrillation in some cases, may be had by, prior to releasing the implant, twisting the delivery device or central core wire such that the radius of the implant is caused to increase. In this way, an initial pressure against the vessel wall may be had (or increased) and an acute treatment efficacy likewise increased. For example, the pushing device may be twisted an angular amount greater than 10° and less than 90°, or, e.g., between about 3 to 5%, the twist having a direction opposite that associated with the helicity of the rings. In some cases, greater or lesser angular amounts may be employed as required.

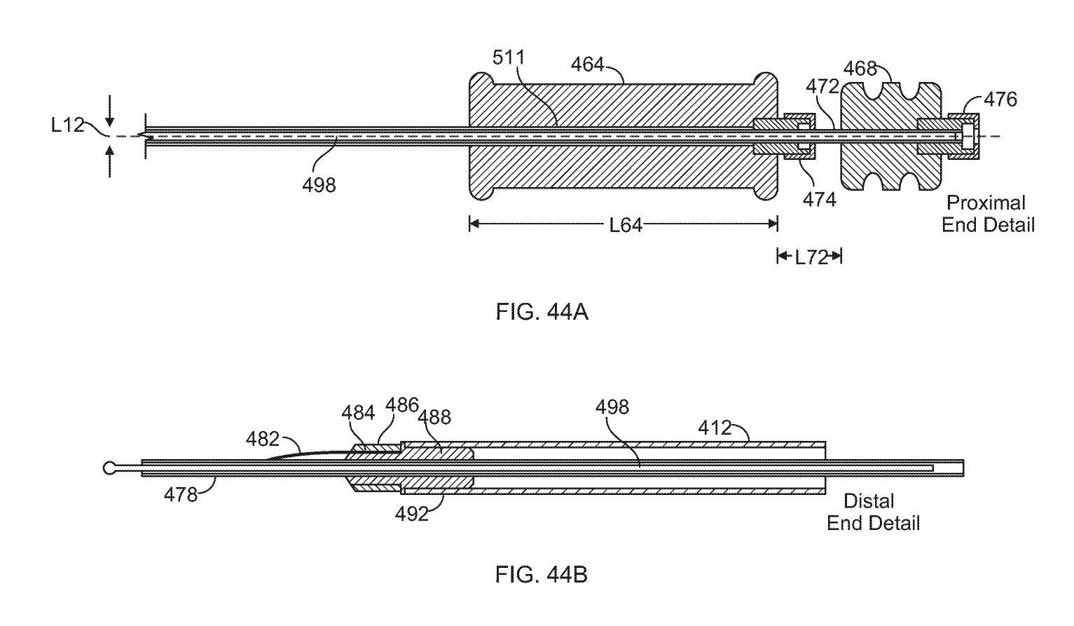

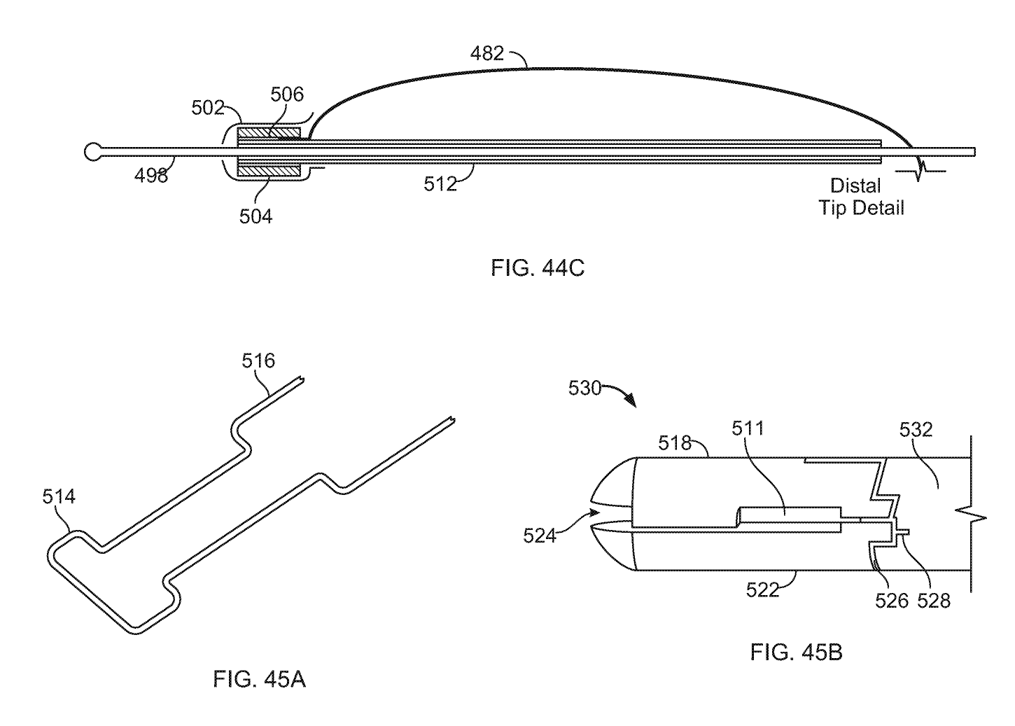

FIG. 40 also illustrates element 466, which along with elements 474 and 476 of FIGS. 44 (A) and 44(B) may constitute Tuohy-Borst hemostasis valves or adaptors.

Referring to FIG. 41, a rectangular lumen 482 may be employed to contain and deliver the implant and a circular or oval lumen 486 may be employed to contain signal wires for the mapping and ablation electrodes. Of course, it will be understood that the shape of the lumens may vary. In this way, mapping may be accomplished prior to deployment of the implant into the vein, e.g., allowing for acute block measurement. Of course, the signal block may not happen acutely in some patients, instead requiring prolonged exposure to the implant. In addition, it will be understood that more than one rectangular or circular lumens may be employed, and their shapes may differ, according to the needs of any given catheter design. In systems where the catheter is made fully steerable or deflectable, additional lumens 484 may be employed to provide the necessary control wires for steering or deflection.

FIGS. 44 (A)-(C) illustrate a related embodiment, as well as various construction and manufacturing details of a specific exemplary version. In these figures, a handle 464 includes a knob 68 which are separated by a distance L72. The distance L72 is chosen to allow for complete deployment of the implant device. A layer of epoxy 511 may seal the handle 464 to the sheath. Referring to FIG. 44 (B), the sheath 496 terminates at a distal end at a distal end bushing 488. A hypo stock sleeve 486 surrounds a layer of epoxy 484 which is used to hold a NiTi tension band 482. The distal end bushing is coupled to the sheath 496 by a layer of epoxy 492. Referring to FIG. 44 (C), greater detail is shown of the distal tip. In particular, a distal end of the NiTi tension band terminates at a hypotube 504 and is held in place by a layer of epoxy 506. A heat shrink 502 is set around the assembly.

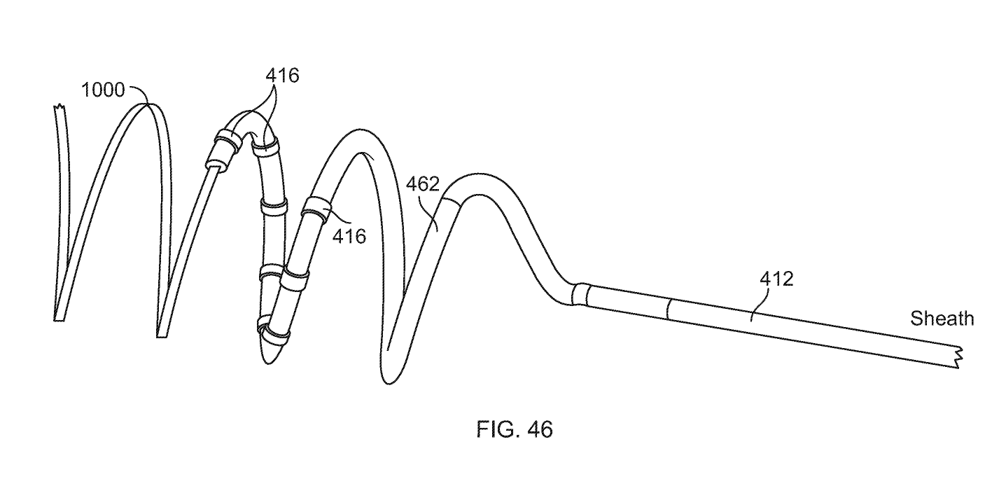

In the above implementation, and referring in particular to FIGS. 40 and 46, the design includes a spiral or pig-tail end that allows the implant to be delivered in a controlled manner and which protects the endocardial surface of the vein. Straight DSCs may also be employed in some configurations. The distal end of the delivery system may be employed for diagnostic purposes, such as ECG mapping of the vein, prior to and after implanting the device, using the electrodes 416. The distal end of the delivery system may further employ similar electrodes for applying RF ablation. The distal end may also allow a user to recapture the implant using devices described below if it is partially or already deployed, enabling further control and proper placement within the PVs.

When delivering the implant, the implant may be pushed by a pusher device through a delivery lumen, and the pusher device may attach to the implant using a grabber mechanism. The pusher device or wire, also just called a “pusher” or central core, may be employed to change the position of the device at least partially within the pulmonary vein. The pusher device or central core wire may include a distal end, the distal end including a device for securing an implant. The device for securing an implant may include a universal joint, the universal joint allowing generally no additional degrees of freedom when the universal joint is within and not adjacent to the catheter distal end, but the universal joint allowing two additional degrees of freedom when the universal joint is outside of or adjacent to the catheter distal end. The device for securing an implant may include a jawbone structure which is closed when the distal end of the pusher is within the delivery lumen and open when the distal end of the pusher is outside the delivery lumen. The implant may include a half dog-bone shape which is inserted within the jawbone structure during the securing. Alternatively, the jawbone may include a boss in a lip of the jawbone, the boss structured and configured such that the implant can only be secured to the jawbone in one configuration. In an alternative implementation, two configurations may be allowed.

The delivery lumen may be configured to allow placement of at least two pushers and two respective implants therein. The delivery lumen may further be configured to allow placement of a cartridge therein, the cartridge containing a plurality of implants.

Referring to FIGS. 45 (A) and (B), the implant may also be held by the catheter by a grabber or grip 530, e.g., a toothed grip. In particular, laser (or other) cuts 526 and 528 may be made in a distal cylindrical catheter tip to form a mouth or grip 524 which may grab the proximal end of the implant. In the figures, the laser cuts are made radially or longitudinally to the cylindrical axis of the grabber. It will be understood that curved cuts may also be employed, according to the needs of the particular application. The cuts allow bending or flexing away from the remainder 532 of the grabber or grabbing means 530. The mouth or grip may be configured, e.g., via heat treatment (e.g., using a memory metal such as nitinol) or design or both, to distend or open when the mouth or grip is not confined by the sheath tube. Once the same is thus extended away from the sheath, the same may open and release the implant.

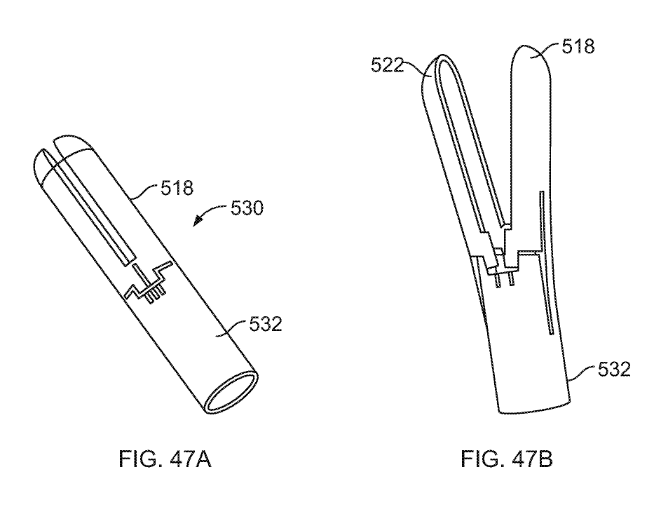

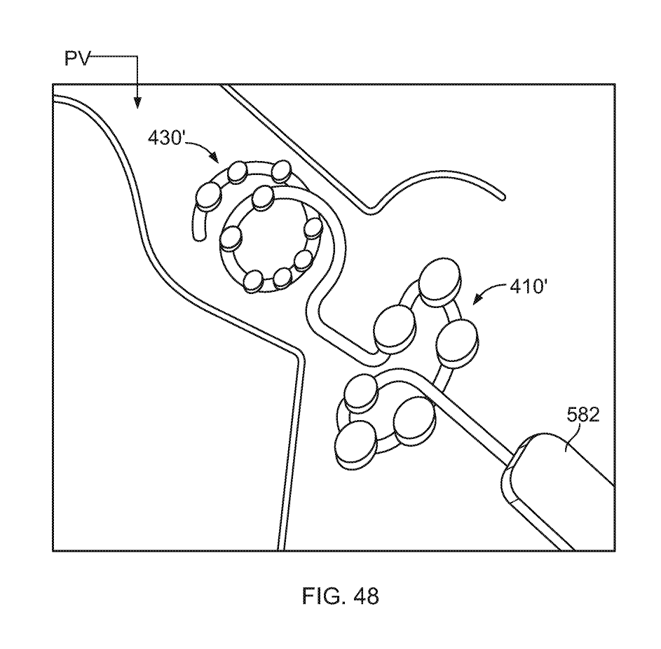

In a related implementation, the implant may be formed with a groove between elements 514 and 516 (see FIG. 45 (A)) or other feature to allow the grabber device 530 to hold the same in a secure and/or locked fashion. Similarly, the grabber device may have formed thereon a “tooth” 511 between upper half 518 and lower half 522 to allow additional points of contact (see FIG. 45 (B)). The scalloped ends of the implant device, described above, may also be employed for this purpose. Additional views are also shown in FIGS. 47 (A)-(B).

In any case, when the grabber device navigates the sheath or delivery catheter, it generally has to navigate both curved sections and straight sections. In some systems, it may be advantageous to provide the same with a small curve or with additional laser cuts to allow the grabber device a degree of flexibility.

A wire may attach the grabber device to the implant to allow the implant to be pulled back if necessary. Activation in the way of electrical energy to the wire may cause the same to break, releasing the implant when in a deployment condition.

Delivery and Deployment Variations

In some implementations, the deployment device, or another device, may allow a degree of recapture to occur in order to fix incorrect implanted device placements within the PV. For example, where the device is pushed through a tube for deployment, the same tube may be used to deliver a small wire equipped with maneuverable jaws at its distal end (such as are shown above in various embodiments). In some cases, for example, a modified guide wire may be employed. A control wire running alongside the guide wire may allow the contraction of one or more jaws in order to grab an errant device. If desired, retraction of the guide wire may then allow the removal of the implanted device. In the system described above where a mouth or grip is closed or opened by virtue of its being enclosed by a sheath or not, respectively, the mouth or grip may be employed to recapture (and redeploy) an implanted device. In the same way, the ratchet sleeve with incorporated balloon may provide this function as well.

In other arrangements, recapture may be by way of a separate device, e.g., a snare. Once ensnared, the device may be reloaded and reinstalled.

Multiple ring devices may be delivered in a single surgical operation, such as in the four pulmonary veins in a given patient. For example, in such a procedure, MRI may be employed initially in order to determine sizes of the various pulmonary veins. According to the order the physician intends to use for deployment, suitable implants may then be loaded into the device. For example, the physician may intend a plan of treatment in a clockwise direction starting with the left superior pulmonary vein, followed by the left inferior pulmonary vein, followed by the right inferior pulmonary vein, followed by the right superior pulmonary vein. The device efficacy may then be verified by performing a pacing and mapping procedure in each vein. That is, conduction block may be verified following deployment, such as by using the mapping capability described in this specification. In general it is desired to measure conduction in the same location both pre- and post-operatively to confirm acute block. If the procedure is surgical, the mapping catheter, e.g., a Lasso®, may be left in place, e.g., exterior of the PV, to ensure the same location of measurement. It is believed to be a particularly beneficial advantage that multiple device deployment and verification may be achieved using a single “stick” through the septum. The above procedure of deployment may only require, e.g., 15 to 20 minutes.

If the pigtail and the implant both have the same helicity, then deployment generally causes the implant to extend and translate longitudinally in the distal direction as it is pushed out. Alternatively, where the sheath is retracted, the implant stays in the same location. However, if the implant and the pigtail have opposite helicity, then the implant will deploy in a proximal direction and may encircle the catheter shaft, which can then be extended or just pulled out as it is. In this way, the implant may be prevented from losing its orientation (axis parallel to the vein) because it is constrained by the catheter shaft.

The above description generally focuses on arrangements where a proximal end of the PVID is coupled to a distal end of a central core. In alternative arrangements, both the proximal and distal ends of the PVID may be coupled to the distal end of a central core or cores (or other such rods). See, e.g., FIG. 25D. In this way, control is gained not just of the proximal end but also of the distal. Consequently, the physician may manipulate the location of the proximal and distal ends of the implant, and may further correct the position and orientation of the device by acts of expanding, pushing, pulling, or rotating.

While the above description has focused on mechanical means to connect the implant to a central core, and thus to be controlled by the same, it will be understood that non-mechanical means of moving a PVID may also be employed, e.g., those not requiring mechanical coupling, e.g., using magnetic fields or the like. In particular, a magnetic force of attraction may be employed to pull a PVID through a DSC, or alternatively a magnetic force of repulsion may be employed to push a PVID through a DSC. Magnetism may further be employed to retract a partially-deployed PVID or even to control and manipulate one that has been deployed and removed from a mechanical connection to the DSC.

While the above description has focused on systems in which a single PVID is loaded and installed at a time, a cartridge system may also be employed in which multiple PVIDs are loaded into a catheter end-to-end or systems in which the ribbons are laid one on top of another, and in which the central core grabs a ribbon similar to the way in which the top piece of paper in a ream is pulled off of a stack to be run through a laser printer.

For surgical delivery, delivery systems may be employed which are in essence large hypotubes. In some systems, a conical shape may be useful, either tapering or expanding in a distal direction, as required by the patient anatomy. Such may allow the implant to be conveniently placed within a vein and expanded by just having the surgeon push the implant through the delivery system.

Mechanism of Operation

Both rings as well as the helix or helices may compress tissue, as to the values disclosed above, stopping the propagation of aberrant signals associated with atrial fibrillation in a manner disclosed. This compression is not necessarily to necrose tissue; rather, the same is to cause a narrowing of certain channels within the tissue associated with the propagation of aberrant electric signals. For example, sodium, calcium, or potassium channels may be blocked by mild compression. The ring(s) may be implanted within a vessel of the heart and may generate circumferential radial pressure sufficient to block the cellular exchange of sodium and/or both sodium/calcium or potassium from entering the cell and thus rendering the cell electrically inert. The ring(s) may apply mechanical pressure to cardiac tissue causing focal apoptosis/necrosis. The ring(s) may have a material composition, surface treatment, coating, or biological agent and/or drug to cause a human biological response, e.g., intimal hyperplasia or endothelization, in a controlled or semi-controlled way in order to effect a long-term electrical block at or within the PV or other electrically active vessels or structures within the heart. It is believed that a suitable amount of force, e.g., as disclosed above, will result in a compression of the first one to five cellular layers in the tissue. In particular, it may be important to at least compress the first layer. Using such a device and method, PV isolation may be achieved without means of an energy source or surgical procedure.

It is noted that the distal ring, inside the PV, as well as the helices, may perform an anchoring function as well as a conductive block function. Moreover, it is noted that a full conductive block is not necessary, nor is full transmurality needed. In some cases, merely a slowing down of the net signal propagation may be enough to frustrate the arrhythmia. For example, a 50% conduction slowing may be highly significant in stopping the propagation of aberrant signals. In any case, the device's geometry, roughly matching the myocardial sleeve, further enhances this effect. It is noted in this connection that throughout the length of the PV, ‘hot spots’ exist where ectopic beats may originate. If the configuration of the ring is such that these are disrupted, then the disruption can act as an efficacious treatment per se. Such disruptions may be particularly effected by the helices between the rings. It is also noted that the ring inside the PV allows for a therapeutic treatment modality in the vein but without the serious complications associated with prior RF or cryogenic in-the-vein treatments, or the like.

It is also noted that the ring may cause the vessel in which it dwells to become more oval or round, or otherwise to maintain a more open shape than that which it adopted before, in the absence of the implant. In this way, the device acts as a stent, enhancing patency and hemodynamics and the resulting blood flow. The device affects the shape of the vein, and vice-versa. This effect improves apposition of the implant to improve outcomes by enabling circumferential contact resulting in conduction block, laminar blood flow, and can help to treat stenotic vessels such as a stenosed PV. One aspect of the device that assists in this regard is the device ring compliance, which causes the device to conform to the vessel—i.e., the radial expansion helps to keep the device in place in a dynamic way, which current PV stents generally cannot. In some cases, the device may be specifically installed to perform the function of a PV stent, and if used in this way, generally, a double-helix design may be employed between the two rings. In some cases single-ring systems may also be employed for such therapies.

It is noted that the above channel-blocking effect of the implant has a multi factorial response mechanism. First is an acute response that, depending on implementation, may last from 1-45 days. After this, depending on the degree to which the implanted device has been treated, a secondary biological or chronic response mechanism may ensure long term block as a result of the biological response to the implant, e.g., endothelialization, the same starting at 15-30 days and lasting indefinitely. The biological response of endothelization cell proliferation is designed to replace myocardial cells or the cells that conduct electrical signals with endothelial cells that are incapable of electrical cell-to-cell conduction. The treatment of the device refers to, e.g., the level to which the device has been roughened so as to act as an irritant to the adjoining tissue. The amount of endothelialization may be ‘tuned’ by this degree of roughening, which may occur via bead blasting, etc. The treatment may also be via surface modification, coatings, or the like. Of course, it is believed that the primary therapeutic effect will be by way of the pressure exerted against the vessel wall.

In some implementations, the metallic nature of the implanted device may be employed to provide a level of active heating so as to heat or necrose tissue adjoining the implant. For example, such heating may be by way of induction or MRI using a device external to the patient. The device may be caused to heat the implant and thus heat (and treat) the tissue creating localized necrosis, and then be easily removed from the vicinity of the patient to stop the heating. In advanced versions of this implementation, the heating device and the implant may be tuned such that only one implant is heated at a time, if multiple implants have been deployed.

Construction

As will be understood, the rings and helices may be constructed of several types of materials. For example, biocompatible metals such as nitinol, cold-worked or heat set, may be employed, and the same exhibit useful shape memory properties. Biocompatible polymers or elastomers may also be employed.

If the ring is made of materials that are bioabsorbable, then the same may eventually be absorbed into the PV by virtue of the endothelialization, leaving only (and at most) a scar visible on the inside of the PV.

The rings may be formed from strips cut from plane of material. Such planes may have a common thickness or may vary in thickness, such as via chemical etching, bead blasting, or other known techniques. An exemplary sheet employable in this way has been disclosed above in connection with FIG. 31. To create the ribbons, the strips may be wrapped around grooves on mandrel, followed by a typical nitinol heat treatment (or alternatively a cold-working treatment). In another implementation, strips may be wrapped around a cylinder, and pins disposed where rings transition to the extension arm. The typical nitinol heat (or cold-working) treatment may then be performed. In a typical nitinol heat treatment, the strip is placed in a 500 to 600° C. fluidized sand bath. The sand bath heat treats the strip such that the austensitic value is set to be about 15 to 20° C. The austensitic value may be altered by tuning the temperature of the sand bath.

Coatings

While not required in any given implementations, various coatings or other agents may be applied or made part of the rings and/or helices, such coatings or agents capable of assisting the disruption of the propagation of aberrant electrical signals or otherwise treating arrhythmias. Such coatings may include drugs, biologics, chemicals, or combinations, and the same may cause some degree of necrosis that by itself or in combination with the mechanical compression acts as a treatment for arrhythmias. For example, a coating including alcohol may be employed as a sort of chemical ablation reagent. Such coatings may also enhance endothelialization as discussed above. As another example, the rings and helices may be coated with tantalum, e.g., a 3-5 micron coating.

A heparin coating may be employed to inhibit thrombus formation. Other coatings may include those that affect conduction within the vessels, including drug-eluting coatings.

Methods of Treatment

One general description of clinical procedures is described below.

When installing the device in a patient, it may be useful to initially measure a level of conduction within the pulmonary vein. Such may be done using electrodes on the catheter delivery device distal tip as indicated above (or using another device). After installation, a second value of the electrical conduction may be measured, and if the second value is not sufficiently below the first, a number of steps may be taken. For example, a touchup ring, e.g., a single ring system, similar to the disclosed implant device but only including one ring, or another implant device like those described, may be installed for additional conduction block. Alternatively, a step may be performed of ablating the pulmonary vein, using RF or cryoablation, using the delivery device or partially-extended implant as described above. In another alternative, the implant device may be reinserted into the pulmonary vein in a different orientation. In yet another alternative, the implant device may be caused to inductively heat so as to cause necrosis or apoptosis of adjacent tissue. The delivery devices described allow for repositioning of the implant without a complete separation of the implant from the delivery device.

Generally in the methods of treatment, implantation of the device provides that the pressure against the pulmonary vein and ostium is substantially consistently greater than zero. The pressure may be constant, or may even increase because, as atrial fibrillation decreases, the pulmonary vein in which the device is implanted is rendered healthier. For example, it is believed that the pressure may increase by 10 to 15% over various time periods. In any case, the necrosis or apoptosis delivered is generally sufficient to block or substantially delay electrical conduction traveling along the axis of the vessel.

After deployment, it has been found to be efficacious if the ring(s) are perpendicular to the axis of the pulmonary vein or within 30° of being perpendicular to the axis of the pulmonary vein. Fluoroscopy may be employed to determine the orientation of the implanted device.

The implant may be permanent, removable, or the same may be configured and designed to be absorbed into the body after a period of time. In a removable embodiment, a removable portion (which may be the entire implant or a portion thereof) may be installed for a period of time, e.g., between 30 minutes and 24 hours, and then removed. During this time, the device may impart pressure against the tissue, necrosing the same and rendering the local tissue electrically inert, thereby creating a block.

Systems and methods may be employed to accomplish treatment of the left atrial substrate, which is also been associated with aberrant electrical signals. Following deployment of all implants, if atrial fibrillation continues, internal or external DC cardioversion may be provided to establish sinus rhythm. RF or cryoablation may also be employed following deployment. The system and method according the principles described here have been associated with enhanced patency of vessels.

Systems and methods according to principles disclosed here may also be employed in valve replacement or repair, treatment of atrial septal defects, or CABG procedures. Other procedures will also be apparent to one of ordinary skill in the art given this disclosure. In cardiac procedures, one such method begins with the cutting of a window into the left atrial appendage, followed by implantation of the PVID through the window, e.g., through a trocar. A stitch may be placed to hold the implant in place if desired, although such is generally not necessary. The window may then be sewn up. An RF procedure may be performed percutaneously, followed by the installation of a touchup coil or ring if indicated.

In percutaneous procedures, a transesophageal probe may be used to check for thrombus, e.g., an ultrasound probe. Vein size may be assessed via e.g., fluoroscopy (by a venogram), and the implant may be chosen to be 1.1 to 1.75 times the vein size e.g., 1.1 to 1.4. Vein size may also be assessed (as well as ovality) using MRI or ICE. MRI may also be employed to check the muscularity of the vein, which may bear on the size of the implant installed: more muscular veins may require larger implants or implants that deliver greater pressures. The femoral vein is accessed by the groin (generally both veins are accessed). A transseptal puncture is performed, and in some cases a physician may dispose an electrode mapping catheter in the coronary sinus or in the high right atrium. The first pulmonary vein generally reached is usually the left superior pulmonary vein, and it is often one of the most active. A clockwise pattern may be performed to implant all of the pulmonary veins. Block may then be checked with an appropriate mapping catheter, e.g., Lasso®. If necessary and indicated, a touchup coil may be installed, or RF or cryoablation may be performed. It is noted that a full block is not always required. A subsequent step of fluoroscopy may be performed to check orientation if indicated. Generally, the PVID should be perpendicular to the vein, e.g., to within 0 to 30°.

Various illustrative implementations of the present invention have been described. However, one of ordinary skill in the art will recognize that additional implementations are also possible and within the scope of the present invention.

For example, the implant may further include a micro circuit formed on the rings or extension arm which is configured to measure or monitor a value of electrical conduction propagating along the axis of the vessel. The micro circuit may be further configured to wirelessly transmit an indication of the electrical conduction. The micro circuit may further be configured to receive an electromagnetic signal and to inductively heat in response to the signal. The micro circuit may also be arranged in a circumferential pattern to provide a mapping capability. The micro circuit may be implemented using a flexible circuit on at least one ring, such as the distal ring or the proximal ring or both. The flexible circuit may include a transmitter for transmitting a wireless signal indicative of the received signals. The transmitter may provide quantitative values of sinus rhythm, or may simply transmit a first type of signal corresponding to sinus rhythm, and a second type of signal corresponding to non-sinus rhythm. The non-sinus rhythm may indicate atrial fibrillation.

The implant and delivery device may be provided in a number of types of kits. The implant including a single or dual ring system with a helical extension arm may be delivered using a standard delivery catheter, or using the catheter system is described herein. Any type of implant which provides such a moderated pressure regime against various vessels or tissues according to the principles described here may be delivered using standard delivery catheters or using catheter systems described herein.

Devices according to the principles disclosed may also be employed on the left atrial substrate, which has also been indicated to be efficacious in the treatment of atrial fibrillation.

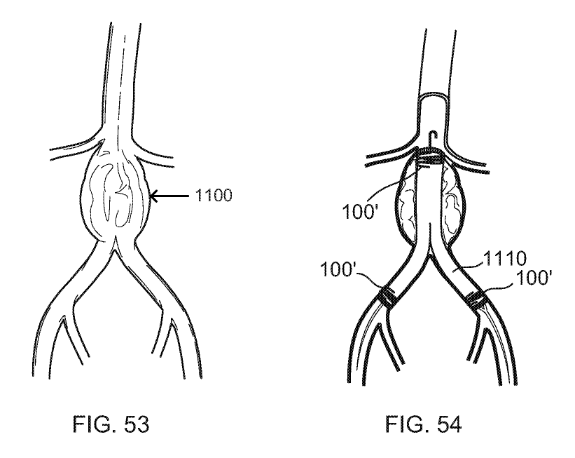

While the procedure and device have been described in the context of the PVs, the same may be conveniently employed in the coronary sinus as well. Other potential treatment sites include the IVC, SVC, coronary sinus, and the vein of Marshall, as well as other vessels and electrically-viable substrates. In addition, the device may be employed to invoke a neurological response of the ganglion plexus. Systems and methods according to the principles described here may be employed to treat abdominal aortic aneurysms (see FIGS. 53-55).

### Alternative Variations

Ablation with Delivery Device, Including with Partial Deployment of Implant

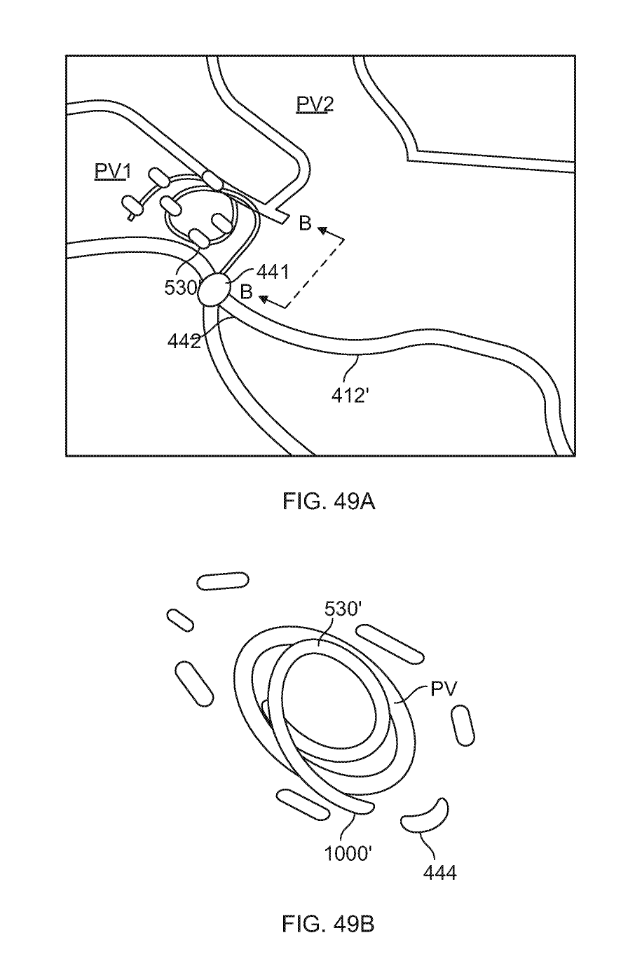

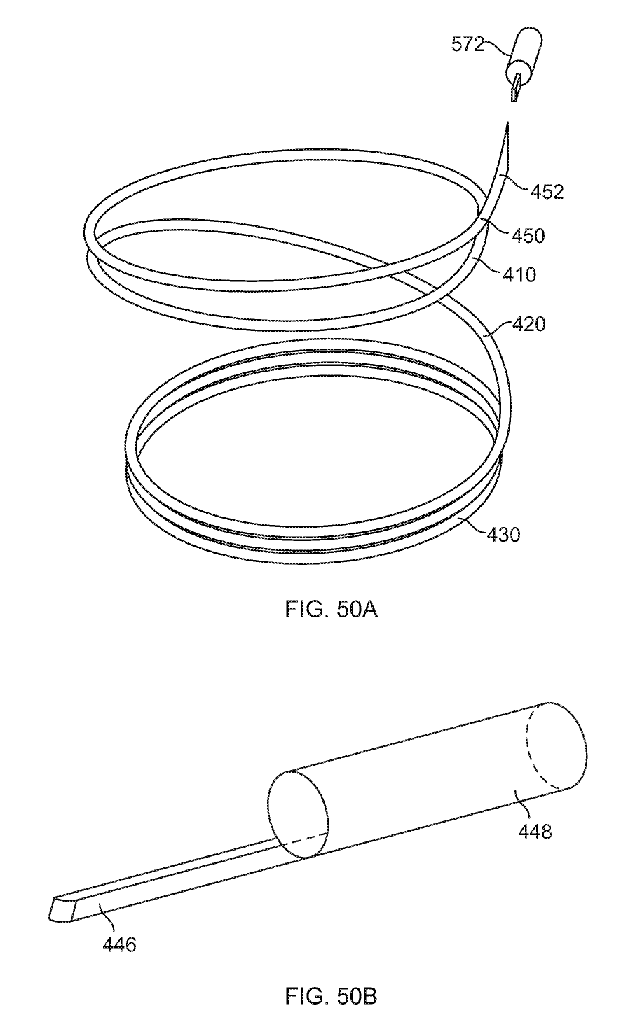

In a related device, and as shown in FIGS. 49 and 50, an ablation device may be provided with a catheter 582 coupled to a proximal ring 510′ and a distal ring 530′. The distal ring 530′ may provide both an anchoring aspect and a mapping aspect. In particular, the distal ring 530′ may incorporate a number of mapping electrodes. The proximal ring 510′ may incorporate a number of ablating electrodes. The distal set may enter into a pulmonary vein and become temporarily apposed to the inner lumen therein. In this sense, the device with two sets of electrodes may be disposed similarly to the implanted device discussed above, but in this case, the same would be retracted after treatment. The distal ring employs its electrodes for mapping, while the proximal ring may employ its electrodes for mapping and/or ablation. The apposed electrode of the distal ring may be as noted above, and while the same may become lodged with respect to translational displacement, the same may also be easily rotated with respect to a track formed by the pressure of the ring against the tissue of the pulmonary vein. The proximal ring electrodes may then contact the ostium and via RF ablation cause necrosis of a ring of tissue around the ostium. In FIG. 50 (A), just one electrode 441 is illustrated, adjacent to where the anchoring pigtail extends into the pulmonary vein. FIG. 50 (B) also illustrates an end-on view of a device 1000′, with a pulmonary vein, a distal ring 430′ within, and dashes 444 indicating the area around the ostium which is ablated. In this system, even without steering, an effective lesion may be creating by rotating the handle and ablating, resulting in a consistent and repeatable lesion that may be created safely. As the same spot is returned to in the ostium, or nearly returned to, by the electrode, or electrodes, a relatively closed-shape lesion is formed and the possibility of micro-reentrant currents is significantly reduced or eliminated. As noted above, the system may conveniently employ some of the same aspects as for the implantable ring system. For example, the cross-section of the ring, or pigtail or spiral, may be rectangular so as to result in a ribbon. A ribbon implementation provides significant translational stiffness while still allowing the system to be retracted back into a catheter. Alternatively, just a portion may be a ribbon, e.g., the distal ring, while the remainder is round, e.g., the proximal ring. Nitinol may be employed as a material for the rings. In this system, therefore, ablation may occur while mapping is also occurring simultaneously. This may be contrasted with prior systems, in which ablating, and testing the results of the ablation, must be performed serially. In this way, ablation may be stopped after a block is detected, minimizing the chance for “over-ablation”.

Of course, in the implementation of FIG. 49 it will be noted that it is not necessary for there to be two separate rings—a continuous set of electrodes may be provided, e.g., to accommodate varying sizes of vessels and cardiac features, and selective electrode activation may be employed to map and/or ablate desired tissue.

In another implementation, an implant device as described may be deployed so as to gain purchase in the PV, e.g., via a partial deployment. The electrodes on the catheter or sheath may then be revolved around the vein by rotating the handle while ablation is conducted at a plurality of locations. In this way, a well-defined circular lesion may ensue, and block may be tested for during the procedure. In this regard, it is noted that one or multiple electrodes may be activated at any one time or during any one procedure. In addition, the user can define circular lesions (by rotating the entire system) or helical lesions (but slowly extending portions of the ring device from the sheath, and revolving the sheath (but not ring device) in so doing). If multiple electrodes are activated while creating a helical lesion, then one can achieve multiple helical lesions, which have in some cases been found particularly useful for atrial fibrillation treatment.

Moreover, following ablation and/or mapping, the ring device may be fully implanted in the vein as described elsewhere. In this way, a multi-pronged technique may be employed to ensure block is achieved and maintained. Of course, in some implementations, the ring device may also be pulled back into the catheter or sheath. In this connection it is noted that the ring device may be permanently attached to the pusher.

Delivery

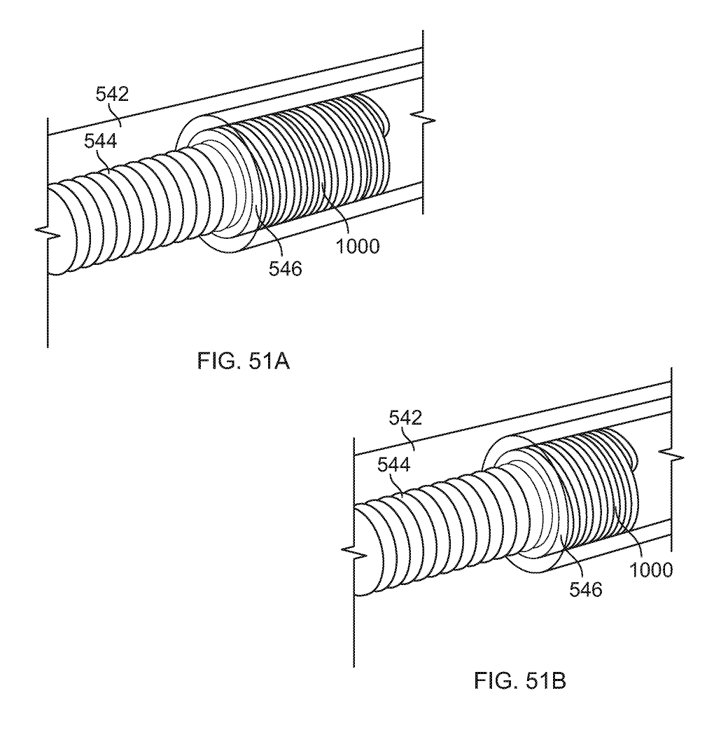

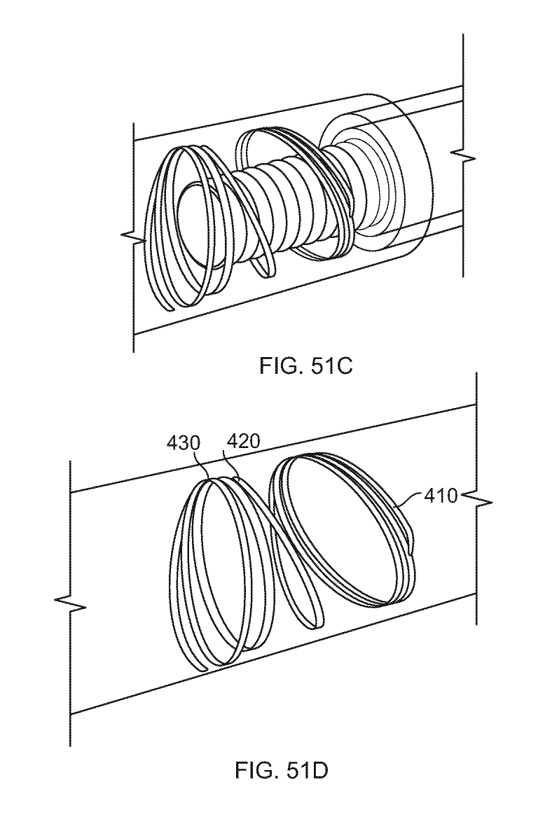

In another implementation for delivery of the device, as seen in FIG. 51, the system may employ a small device, i.e., a ratchet sleeve having a cylinder 448 and extension 446, within the delivery catheter or sheath that can provide a ratcheting function. In this way, the handle may be simplified, and provided with greater control, by having the operator only have to provide a repeated short-stroke motion to controllably cause the implant to exit the sheath and become implanted in the PV. In other words, once the implant is pulled back into the sheath, and the ratchet sleeve is disposed near the distal tip of the sheath, then the implant may be deployed by repeatedly pushing it out of the tip, e.g., a fraction of a centimeter, e.g., a ¼ centimeter, to 2 inches, at a time. The implant is prohibited against retracting into the sheath by virtue of the ratchet sleeve.

In a further related embodiment, a small balloon may be inflated within the ratchet sleeve if desired to provide a way for the ratchet sleeve to grab onto the implant. By placing a tip of the implant, e.g., the proximal tip, into the ratchet sleeve, and inflating the balloon to fill up the interstitial space, the implant may be effectively grabbed by being held between the balloon and the wall of the ratchet sleeve. In another embodiment, the inflation lumen and balloon may be provided in the pusher, and the device may be grabbed by inserting the pusher into the ratchet sleeve and inflating the balloon, thereby constricting the implant tip in the same small diameter as the balloon (within the ratchet sleeve), causing the same to be grabbed. In yet another embodiment, a small balloon may be employed to render the volume within the ratchet sleeve closed, and in that case a small negative pressure may be pulled on the interior of the ratchet sleeve, constricting its walls and causing the same to pull inwards, grabbing onto the implant in the process.

In an alternative implementation, illustrated in FIGS. 51 (A)-(D), the implant device 1000 is coiled around a threaded mandrel 544 and confined by an outer tube 546. Removal of the outer tube allows the implanted device to spring away from the mandrel by virtue of its shape-memory character. FIGS. 51 (A)-(D) illustrates a sequence of deployment steps. In general, removing the outer tube causes immediate deployment, resulting in impingement of the device 1000 against a vessel wall 542.

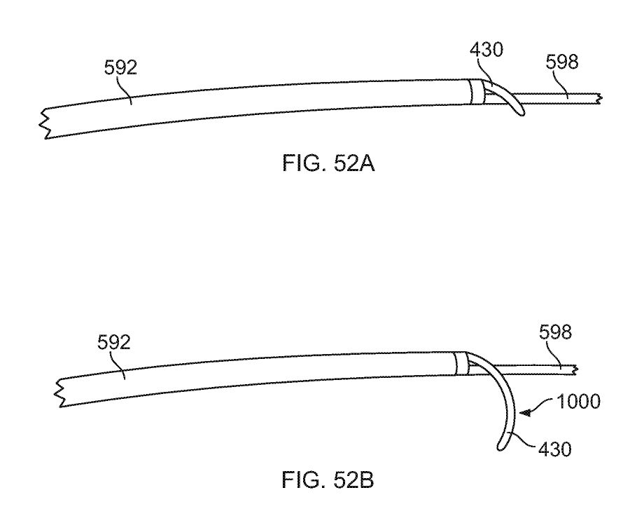

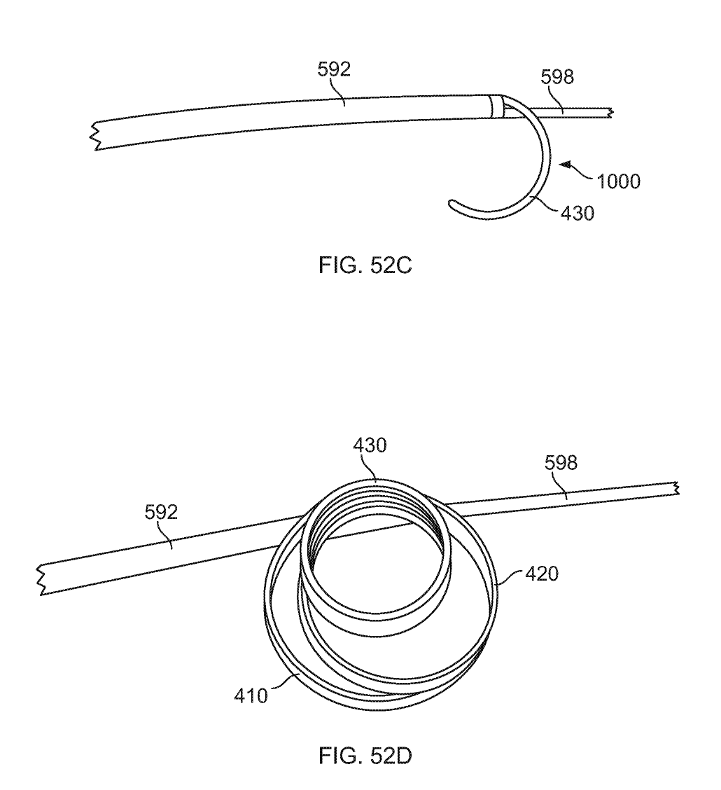

FIGS. 52 (A)-(D) illustrates another embodiment, also illustrating a sequence of deployment steps, in this case which deploys the implant perpendicularly to the direction of implantation of FIGS. 51 (A)-(D). This deployment direction may be useful in certain patient anatomies. In FIGS. 52(A)-(D), the implant 1000 emerges directly (and initially linearly) out of the distal tip of the catheter 592. The distal ring 430 emerges first, followed by the proximal ring 410. In this embodiment, a pusher may be employed, or, e.g., the grabber or central core wire disclosed above. Generally, the implant will be held stationary relative to the patient, and the delivery device moved in a proximal direction to slowly uncover or reveal the implant, and thus cause the same to wind into a deployed configuration. Depending on the design of the implant, rather than deploying as shown in FIG. 52, a deployment variation may take advantage of a natural tendency of the implant to self-right, i.e., naturally adopt an orientation collinear with the vein.

In various implementations, the implant may be deployed from the proximal side first, such as at the ostium of the atrial/vein junction, followed by deployment of the distal ring within the vessel. This is advantageous as more mechanical force can be applied to the luminal surface of the myocardial sleeve. In particular, the first ring may be disposed in the ostial/atrial junction location, implanted, and the helices and second ring may then be unwound or uncoiled around and into the PV. This unwinding or uncoiling deployment allows installation of an implant that can provide sufficient mechanical force to achieve the clinical response necessary to create conduction block, e.g., destruction of cell coupling at the gap junction/connexin level at the intercalated disc, as well as inactivation of the Na-channels, causing dehydration of the cells by compression, resulting in conduction block and vein isolation. It is noted in this connection that a set of rings, connected by helical extension arms, sized for the vein, but allowed to simply expand, such as by the effect of the shape memory alloy, may in certain cases not provide the needed mechanical force to compress the surface cells. In addition, during deployment, e.g., while the implant is partially deployed, the action of the partial implant on the electrical signal propagation may be confirmed or verified to check the level of isolation achieved.

To deploy the distal end first, a split catheter shaft may be employed, such that separation of the catheter shaft at a location near the distal end causes the distal end to be deployed first. Of course, in certain implementations, the proximal end may also be deployed first. Such a split catheter shaft may be employed, e.g., in the delivery of the implant shown in FIGS. 19 (A)-(D). In this implementation, the distal end of the catheter may employ a polymer tip for atraumatic delivery, and the polymer tip may be radiopaque. As in most of the implementations described, the catheter may be delivered over a guide wire.

In another implementation, the distal end of the device is sutured to the catheter, and the wire of the device is wrapped around the catheter. In this connection it is noted that the implant, during delivery, undeployed and constrained in a delivery device, may take the form of a straight wire, a helically-wrapped wire, or another configuration. The sutured end causes the distal end to be deployed last, and the final separation of the distal end from the catheter may be effected by way of cutting using a blade configured for that purpose, an electrical arc, or the like.

In general, the delivery system will have distal and proximal ends, where the distal end employs an atraumatic distal tip and the proximal end includes a handle. The system further includes a catheter shaft having a tubular structure traversing from the proximal end to the distal end. The guidewire lumen includes a luminal space to enable passage of a range of guidewire sizes. In one implementation, the guidewire lumen is furthermore capable of being advanced distally or proximally to enable deployment of the coil-like implant attached along the external surface of the guidewire lumen and contained within the inner surface of the outer catheter shaft. As in some embodiments above, the DSC may employ a flexible distal segment and a steering wire anchored at the distal portion of the delivery catheter.

As noted in many delivery systems it may be desired to hold both ends of an implant during deployment, and then to release the ends once a desired location is determined. Such systems also allow a degree of manipulation to be usefully retained by the physician during deployment, such that each end of the implant (as well as the rest of the implant) is at a desired location. Moreover, control of the ends of the implant generally allows rotation of the implant to occur, which can provide additional features such as additional therapeutic pressure against the vessel wall. Reduced pressure may also be provided in this fashion, at least temporarily, such as may be desired for movement of the implant. Control of both ends of the implant further allows less stress to be placed on the implant during deployment. In addition, it has been found that deploying such an implant out of a sheath is made easier when both ends are controlled. The physician can push the implant, pull the implant, telescope the implant, twist the implanted, decrease its diameter (e.g., for ease in moving the implant), increase its diameter, and the like.

One challenge is to provide such capabilities within a low profile delivery system, e.g., 11 French (although other delivery system sizes may also be employed, including both larger and smaller delivery systems). Larger delivery systems also allow for employment of a central lumen, not only for guide wires, but also for diagnostic, analysis, or mapping catheters to be delivered therethrough. Such may be conveniently employed while an implant is still controlled by the delivery system to determine efficacy. If insufficient, the implant may be manipulated to increase the therapeutic effect.

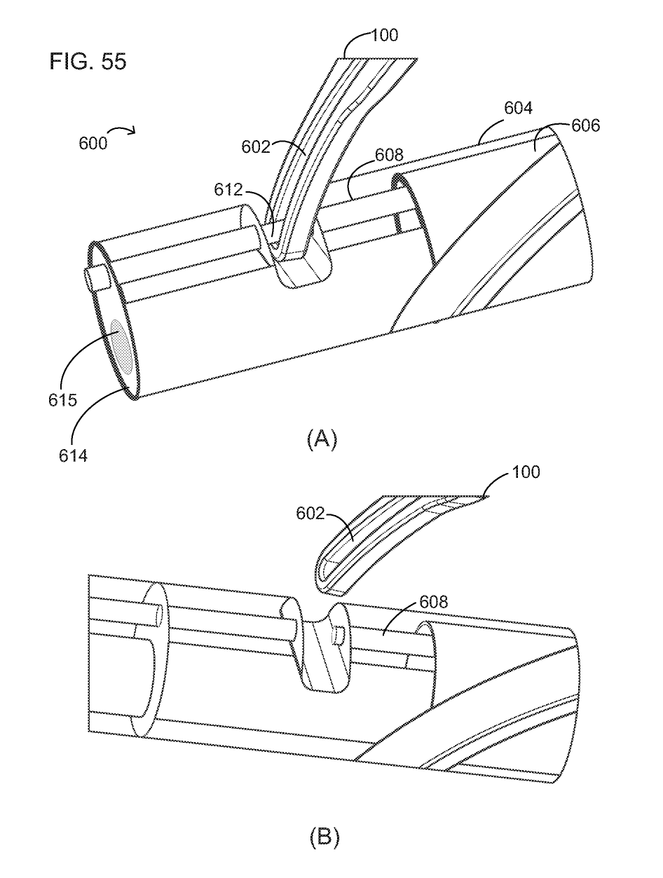

FIGS. 55-62 illustrate such a low profile delivery system. In these figures, a system 600 is illustrated in which a PVID implant device 100 is temporarily mounted for delivery. The implant device 100 may include single, dual, or multi-ring systems. In the system 600 of FIG. 55, proximal and distal ends of the implant require a degree of twisting to be inserted within a shaft 604, and to ease such twisting, a void 602 may be defined by the implant 100, which makes the end of the implant easier to rotate with respect to an axis of symmetry defined by an outstretched length of the ribbon. The void 602 also allows convenient placement of a wire 612 for accepting the end of the implant and securing the same against movement during delivery and deployment. The wire 612 may travel through a wire shaft 608 which in turn travels through an inner shaft 606 within the outer shaft 604. Also within the outer shaft 604 is a guide wire shaft 614 which defines a guide wire lumen 615. The outer diameter of the lumen 615 may be chosen such that not only a guide wire but also various diagnostic, mapping, or other such catheters may be disposed therethrough.

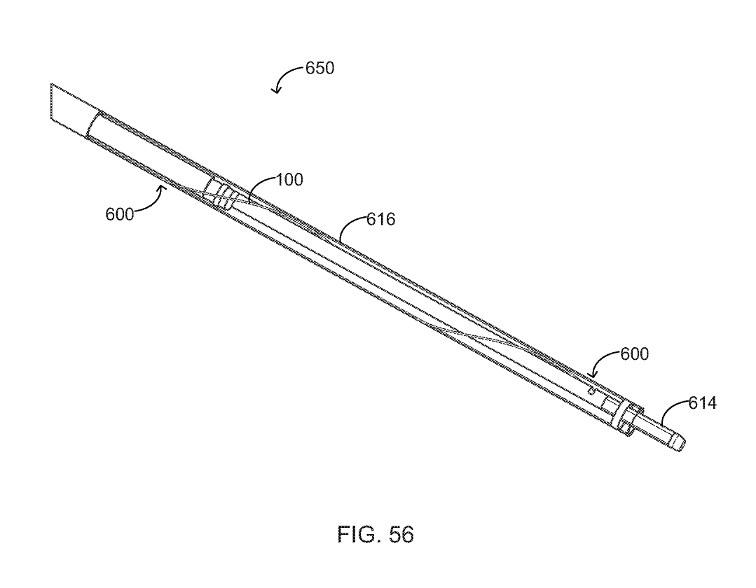

FIG. 55A illustrates the wire 612 holding the implant 100 secure, and FIG. 55B illustrates the wire 612 being retracted and the implant 100 being released. Referring to FIG. 56, a complete system 650 is illustrated in which both ends of the PVID implant 100 have an attachment system 600 associated. The attachment system 600 allows independent attachment and detachment of each end of the PVID implant 100. The shaft 614 is also illustrated in FIG. 56. A protective shaft 616 is illustrated in the figure, the shaft 616 serving to encase and protect the PVID implant 100. By protecting the implant in this way, and providing independent means to detach the ends of the implant during deployment, the system may be conveniently manufactured and sold as a unit.

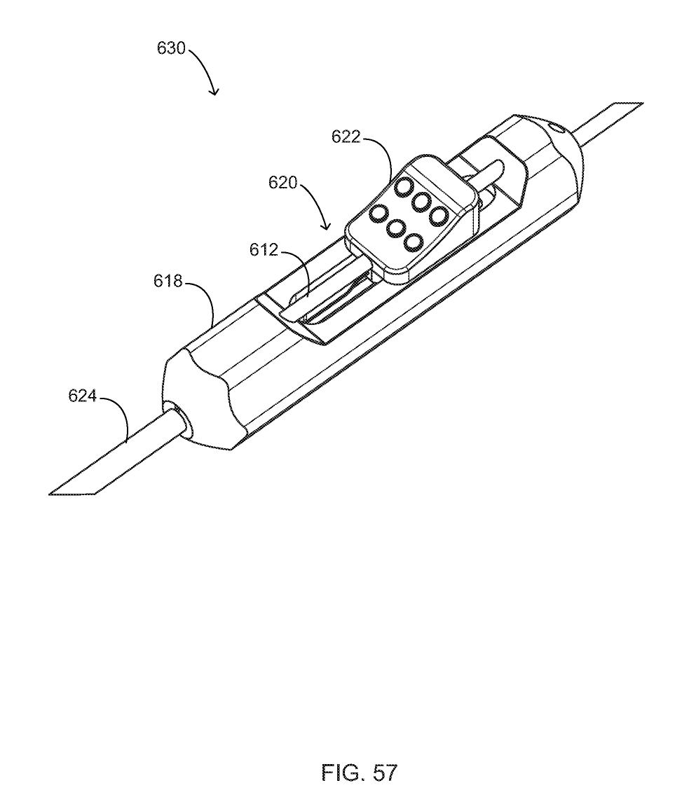

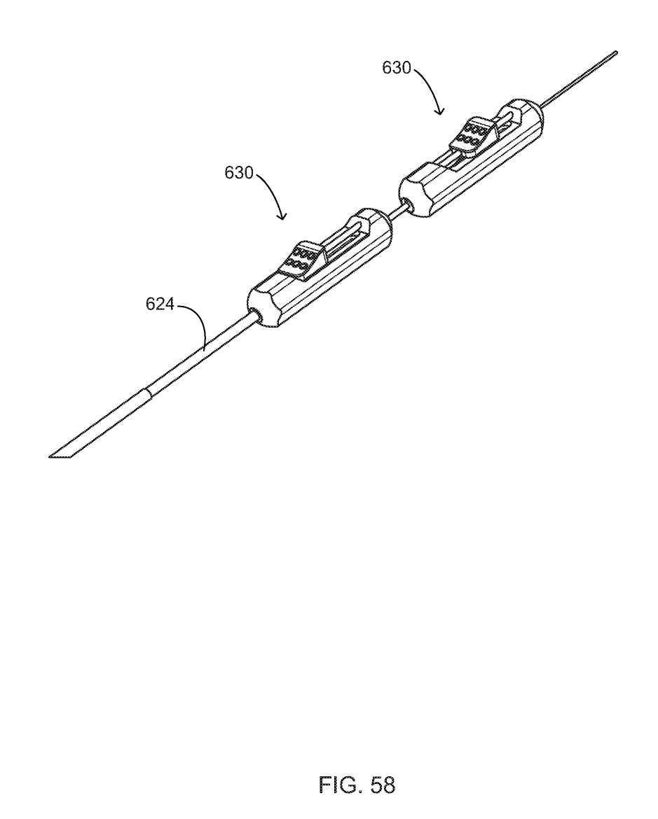

One independent means of detachment is illustrated by the assembly 630 of FIG. 57. In the assembly 630, a shaft 624 is illustrated which may couple to the outer shaft 604 or may be integral therewith. A handle 618 is provided in which a void 620 is defined, the void 620 allowing constrained movement of a slider 622 attached to the wire 612. When the slider 622 is forward, i.e., to the left in the figure, the wire 612 constrains the implant 100 against release. When the slider 622 is moved to the right in FIG. 57, the wire 612 moves to the right and the implant is no longer constrained and thus released. A suitable amount of travel may be, e.g., ½ to ¾ of an inch. FIG. 58 illustrates a proximal end of a delivery system. The sliders and handle assemblies 630 may be sold along with the shaft 624 and the implant as a single sterile unit. Additional components may be employed to provide a complete system or the same may be inserted through, e.g., an introducer sheath.

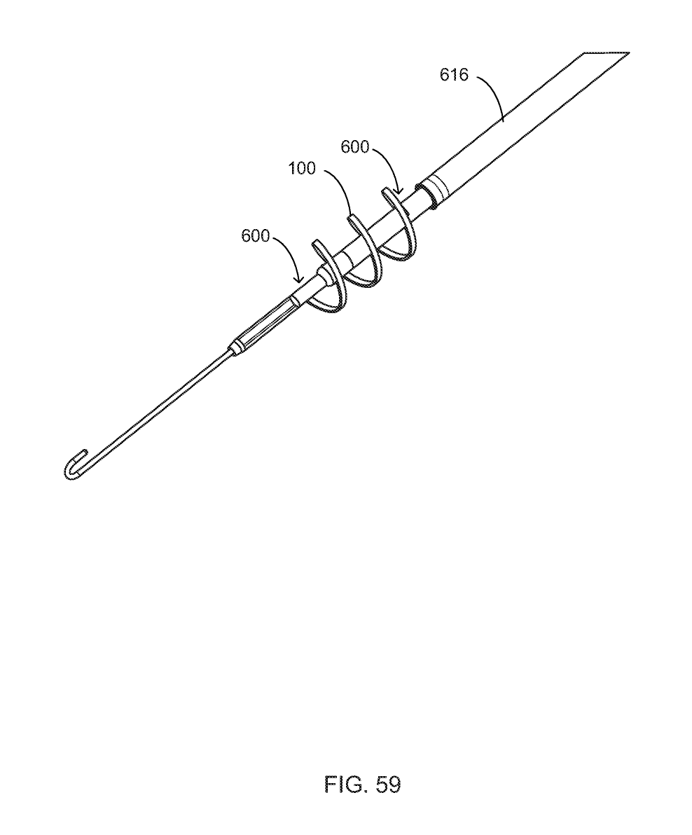

Referring to FIG. 59, an implant 100 is illustrated with the protective shaft 616 retracted but the implant 100 only partially deployed. The shape memory material of the implant 100 allows its expansion once the protective shaft 616 is retracted, once the proximal end of the implant and the distal end of the implant are moved towards each other, at least in relative motion. Once the implant is in position for deployment, the wires 612 may be retracted.

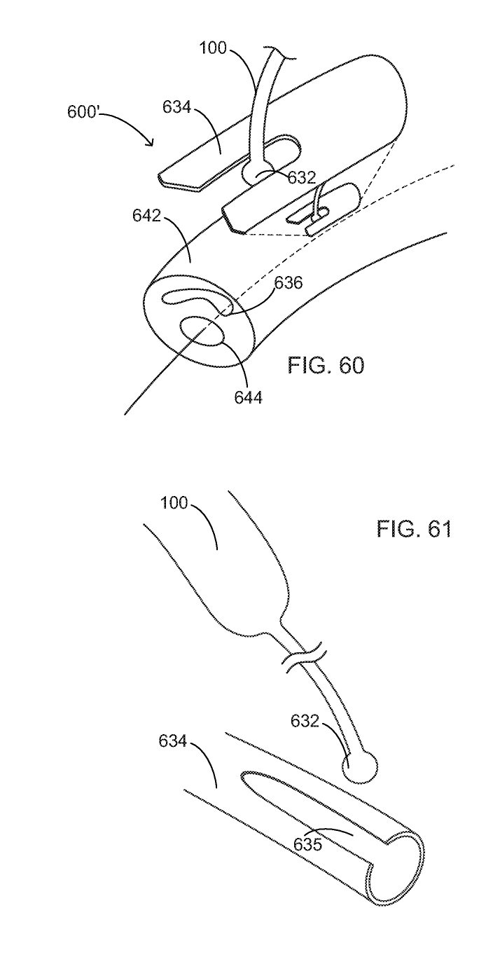

FIGS. 60-62 illustrate an alternative but related delivery mechanism 600′, where an implant 100 has a ball end 632 disposed at its proximal and distal ends. The ball end 632 engages in a void 635 formed in a forked end defined by engagement shaft 634. The engagement shaft 634 may move within a lumen 636 within an outer shaft 642, with a hole defined within the shaft 642 to allow the ball end to be disengaged and released by retraction of the forked end of the engagement shaft 634. A guide wire lumen 644 may also be defined within the shaft 642. FIG. 62 illustrates engagement of the implant 100, and more particularly a proximal or distal end, with the mechanism 600′.

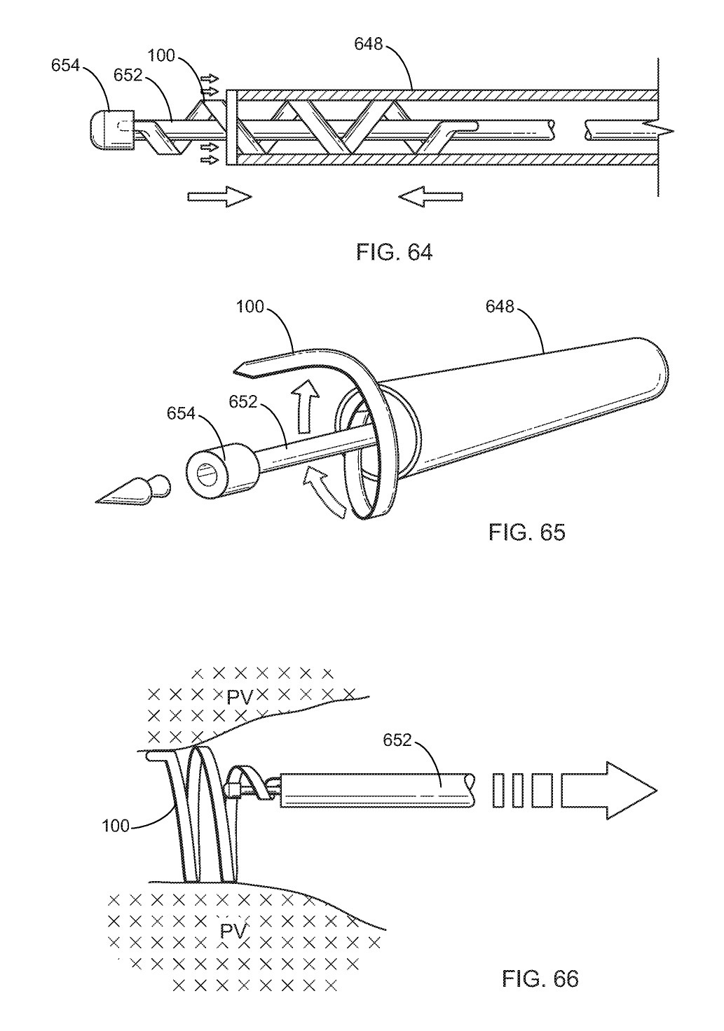

FIGS. 64-66 illustrate an alternative but related delivery mechanism, in which an implant 100 encircles a shaft 652 and is friction fit to a cap 654. The implant, shaft, and cap move within an outer shaft 648. During deployment, distal movement of the shaft 652 longitudinally translates the implant 100 into a deployment location. Further movement of the shaft 652 causes the distal end of the implant 100 to disengage from its frictional fit with the cap 654 because the implant may be constrained against further distal movement by a backplate or by securing to an interior shaft (not shown). In other words, it disengages from the cap because it is in essence pulled out from the same. Once disengaged, the distal end of the implant 100 expands as illustrated in FIG. 65, and may engage a pulmonary vein as shown in FIG. 66.

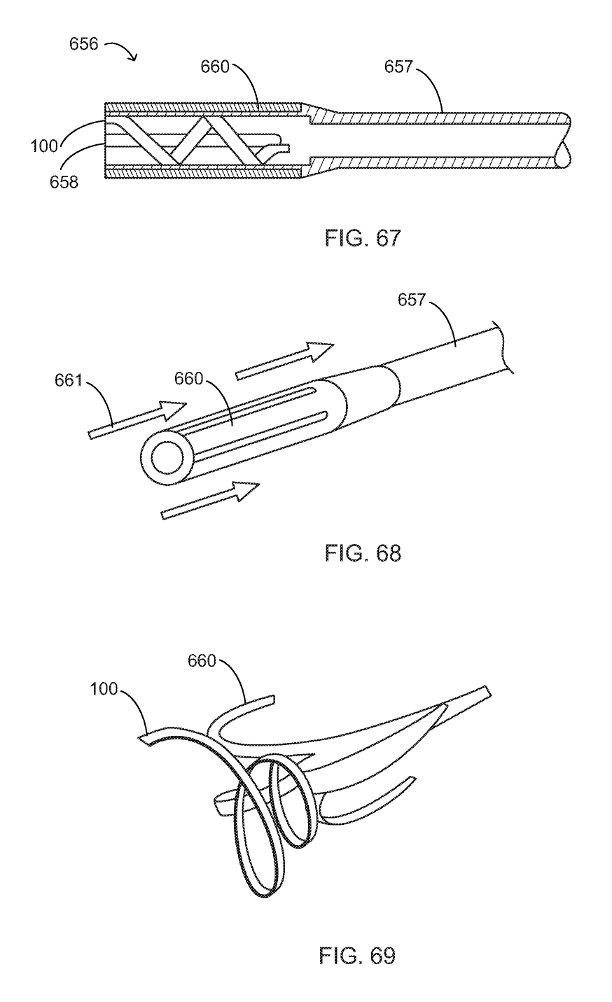

Yet another alternative delivery mechanism 656 is illustrated in FIGS. 67-69. The system 656 includes a shaft 657 with a frangible cylindrical section 660. Within the shaft 657 and section 660 may be the implant 600 encircling an inner shaft 658. In this case, the inner shaft 658 is optional. The different frangible portions within the section 660 may be separated at a distal end or may be connected by thin strips of material. By pushing or providing another force in the direction indicated by arrows 661, the frangible sections may separate and be displaced in a manner similar to a banana peel, as shown in FIG. 69. Such displacement allows release of the implant 100.

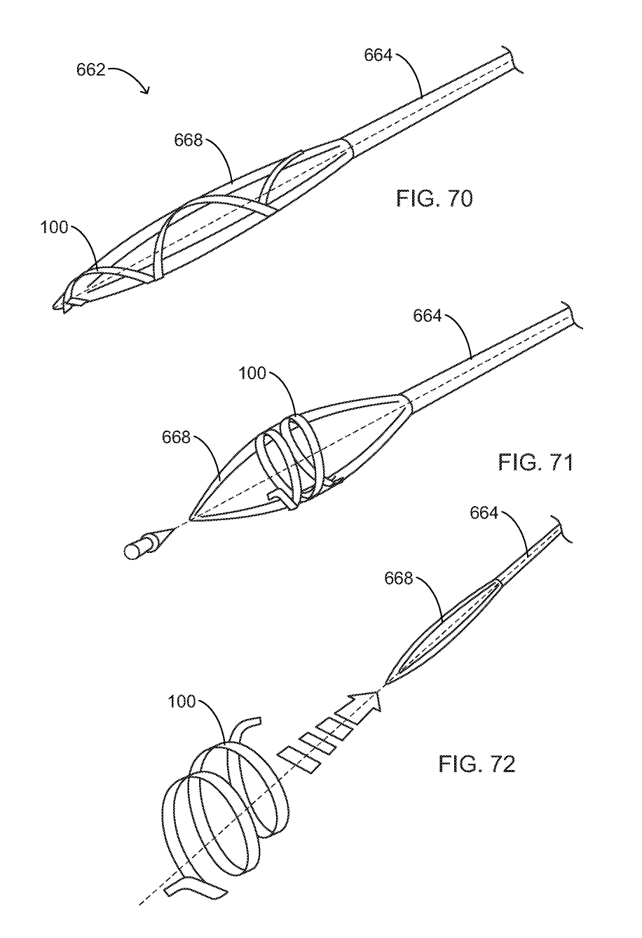

Yet another alternative delivery mechanism 662 is illustrated in FIGS. 70-72. In these figures, a PVID implant 100 encircles a balloon 668 which is coupled to a delivery shaft 664. Inflation of the balloon is illustrated in FIG. 71; deflation and withdrawal of the balloon is illustrated in FIG. 72. The balloon 668 is generally not required for expansion of the implant 100. However, by expanding the implant and further expanding the implant and walls of the vessel using the balloon 668, it is believed that the therapeutic effect can be even further enhanced.

One of ordinary skill in the art given the description above will understand that numerous variations of the disclosed systems and methods are within the scope of the invention. For example, referring to FIGS. 53 and 54, an implementation may be employed in the treatment of an abdominal aortic aneurysm 1100. As is known, various prosthetics PTFE sleeves have been proposed for the treatment of abdominal aortic aneurysms, such sleeves having a proximal portion within the aorta and “legs” in the iliac arteries. Support for such sleeves has been known in the prior art by way of wireframes or the like. In FIG. 54, a sleeve 1110 is illustrated which is held in place by a single ring systems 100′ as have been described above. Such ring systems may be entirely within the sleeve, and hold sleeve in place using radial outward pressure, or may have a portion outside of the sleeve, and in part maintain patency of the sleeve by eventually be integrated into the aortic wall. Such systems may provide a convenient treatment for AAA maladies.

In another alternative implementation, a surgical robot may be employed to assist in the delivery of the implant to the one or more pulmonary veins, e.g., using robot surgery systems developed by Hansen®, Intuitive Surgical®, and the like. In addition to assisting in the disposing of the distal tip of the DSC at an appropriate location, e.g., the pulmonary veins, a robot system may be employed to perform the retraction and (if necessary) rotation necessary to deploy the implant. An algorithm may be employed which is run at the time of deployment. The algorithm may cause the robot to retract and rotate the delivery system, e.g., relative to the central core, in order to deploy the implant. Inputs to the algorithm may include the length of implant, the desired pitch of the implant, the type of implant (single or dual ring) and the desired orientation, e.g., amount of desired perpendicularity to the vessel axis. The algorithm may accept data from a venogram or MRI or the like and automatically calculate desired delivery parameters using such information.

In yet another implementation, the ring system may be employable as a structure on which an artificial valve system is constructed. For example, the implant device may be placed in the vasculature where a valve is desired, and the valve may be held in place by the implant device and may fill the volume within the interior of the implant device, i.e., within the helical coils.

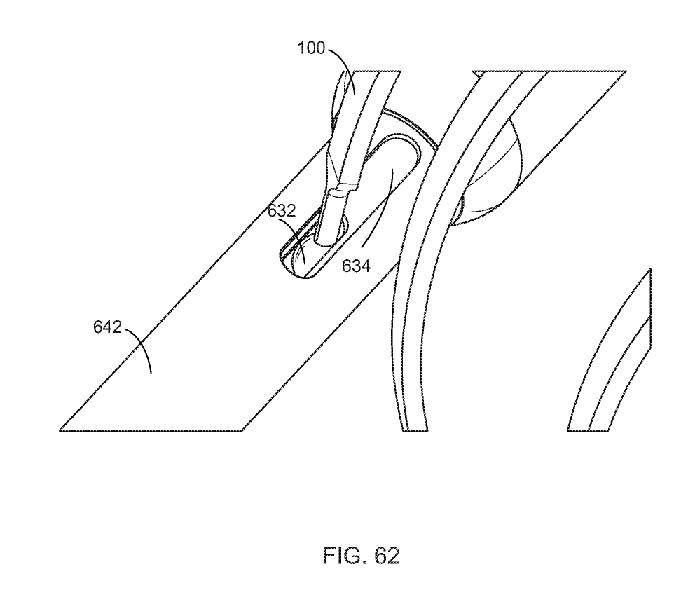

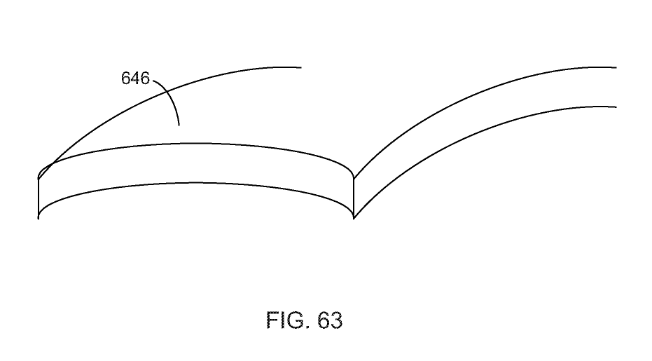

The proximal and distal ends of the implant may be given any number of shapes, besides those illustrated above in, e.g., FIGS. 37-39. For example, proximal or distal ends of an implant ribbon may be in the shape of a “T”, a bulb, an asymmetric bulb, a series of ratchets, or the like. Besides ribbons having rectangular cross-sections, ribbons having curved cross-sections may also be employed, e.g., as is illustrated by the ribbon 646 in FIG. 63. Various other cross-sectional shapes for the ring windings may also be employed.

Other variations will also be understood. Accordingly, the invention is to be limited only by the claims appended hereto.

1. An implant device for treating atrial fibrillation, comprising:
a. a ring formed of at least one winding of a ribbon, wherein the width of the ribbon is between about 0.5 and 2.5 mm;
b. wherein in an undeployed configuration the diameter of the ring is between about 10 to 60 mm and wherein in a deployed configuration the diameter of the proximal ring is between about 10 to 30 mm;
c. and wherein the ring is configured to deliver a pressure against adjacent tissue when deployed of between about 0.01 N/mm² and 1.4 N/mm².

2. The implant device of claim 1, wherein the ring includes at least 1.5 windings of the ribbon.

3. The implant device of claim 1, wherein the ring is configured to deliver a force against adjacent tissue when deployed of between about 0.02 N/mm² and 0.4 N/mm².

4. The implant device of claim 1, wherein the width of the ribbon is between about 0.25 and 2 mm.

5. The implant device of claim 1, wherein the implant device is coated with a material composition, surface treatment, coating, or biological agent and/or drug.

6. The implant device of claim 1, wherein two rings are provided, each ring formed of at least one winding of a ribbon, wherein the width of the ribbon is between about 0.5 and 2.5 mm, wherein in an undeployed configuration the diameter of the ring is between about 10 to 60 mm and wherein in a deployed configuration the diameter of the proximal ring is between about 10 to 30 mm, and wherein the ring is configured to deliver a pressure against adjacent tissue when deployed of between about 0.01 N/mm² and 1.4 N/mm², and wherein between the rings and extension arm is provided.

7. The implant device of claim 1, wherein a total force provided against adjacent tissue is between about 0.2 N and 10 N.

8. The implant device of claim 7, wherein a total force provided against adjacent tissue is between about 0.2 N and 6 N.

9. The implant device of claim 1, wherein the ribbon has a proximal end and a distal end, and wherein the proximal end and the distal end each define a hole therein.

10. The implant device of claim 1, wherein the ribbon has a rectangular cross-section.

11. The implant device of claim 1, wherein the ribbon has a curved rectangular cross-section.

12. The implant device of claim 1, further comprising a micro circuit disposed on the ribbon.

13. The implant device of claim 12, wherein the micro circuit is configured to wirelessly transmit a signal corresponding to a detected heart signal.

14. The implant device of claim 13, wherein the heart signal is indicative of heart rhythm.

15. The implant device of claim 14, wherein the heart signal is an EKG.

16. A kit for treating a malady by deploying an implant device in a vessel, comprising
a. the implant device of claim 1; and
b. a delivery system.

17. The kit of claim 16, wherein the delivery system includes one or more electrodes on a distal end, the one or more electrodes for mapping, pacing, or ablating.

18. The kit of claim 16, wherein the delivery system comprises:
a. first means for attaching a proximal end of a ribbon to a first shaft;
b. second means for attaching a distal end of the ribbon to a second shaft, the second shaft movable with respect to the first shaft;
c. third means for releasing the proximal end; and
d. fourth means for releasing the distal end.

19. The kit of claim 16, wherein the kit includes at least four implant devices, each according to claim 1.

20. A method for treating a malady, comprising:
a. choosing a size of an implant device for insertion into a vein of a patient, the implant device including a ring formed of one or more windings of a ribbon, the ribbon having a width of between about 0.1 and 2.5 mm, and a diameter of between about 12 and 50 mm; and
b. inserting the implant device into the vein of the patient,
c. such that the choosing includes selecting the size of the implant device such that the implant device applies a pressure against in adjacent tissue, the pressure between about 0.01 N/mm² and 1.4 N/mm².

21. The method of claim 20, wherein the malady is atrial fibrillation.

22. The method of claim 20, wherein the choosing includes selecting the size of the implant device such that the implant device applies a pressure against in adjacent tissue, the pressure between about 0.02 N/mm² and 0.4 N/mm².

23. A delivery device, comprising:
a. first means for attaching a proximal end of a ribbon to a first shaft;
b. second means for attaching a distal end of the ribbon to a second shaft, the second shaft movable with respect to the first shaft;
c. third means for releasing the proximal end; and
d. fourth means for releasing the distal end.

24. The delivery device of claim 23, wherein the second means includes a shaft and a cap and wherein a frictional fit is provided between the distal end of the ribbon and the shaft and the cap.

25. The delivery device of claim 23, wherein the first and second means are wires that engage holes defined in the proximal and distal ends of the ribbon.

26. The delivery device of claim 24, wherein the third and fourth means are sliders that engage the wires.

27. The delivery device of claim 23, further comprising a shaft defining a guide wire and delivery lumen.

28. The delivery device of claim 23, wherein the delivery device further comprises a distal tip, and further comprising electrodes disposed on the distal tip.

29. The delivery device of claim 28, wherein the electrodes are configured for mapping or ablating.

30. The delivery device of claim 23, wherein the third and fourth means are coupled, such that the proximal end and the distal end are released at substantially the same time.
